SEQUENCE LISTING (82 sequences)

SEQ ID NO: 1 - protein, 259 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 2 - protein, 19 aa, synthetic construct
MDWTWRILFLVAAATGAHS

SEQ ID NO: 3 - protein, 278 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 4 - protein, 4 aa, synthetic construct
RAAA

SEQ ID NO: 5 - protein, 22 aa, synthetic construct
GSGATNFSLLKQAGDVEENPGP

SEQ ID NO: 6 - protein, 20 aa, synthetic construct
MYRMQLLSCIALSLALVTNS

SEQ ID NO: 7 - protein, 30 aa, synthetic construct
MAPRRARGCRTLGLPALLLLLLLRPPATRG

SEQ ID NO: 8 - protein, 377 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 9 - protein, 425 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 10 - protein, 205 aa, synthetic construct
ITCPPPMSVEHADIWVKSYSLYSRERYICNSGFKRKAGTSSLTECVLNKATNVAHWTTPSLKCIRSGGGSGGGGSGGGGSGGGGSGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 12 - protein, 21 aa, synthetic construct
MALLLALSLLVLWTSPAPTLS

SEQ ID NO: 13 - protein, 77 aa, synthetic construct
GTNDAEDCCLSVTQKPIPGYIVRNFHYLLIKDGCRVPAVVFTTLRGRQLCAPPDQPWVERIIQRLQRTSAKMKRRSS

SEQ ID NO: 14 - protein, 520 aa, synthetic construct
MYRMQLLSCIALSLALVTNSATSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGSGATNFSLLKQAGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 15 - protein, 884 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAPRRARGCRTLGLPALLLLLLLRPPATRGITCPPPMSVEHADIWVKSYSLYSRERYICNSGFKRKAGTSSLTECVLNKATNVAHWTTPSLKCIRSGGGSGGGGSGGGGSGGGGSGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGATNFSLLKQAGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 16 - protein, 10 aa, synthetic construct
GDSVSSNSAT

SEQ ID NO: 17 - protein, 9 aa, synthetic construct
TYYRSKWYN

SEQ ID NO: 18 - protein, 13 aa, synthetic construct
ARGMMTYYYGMDV

SEQ ID NO: 19 - protein, 9 aa, synthetic construct
SGINVGPYR

SEQ ID NO: 20 - protein, 7 aa, synthetic construct
YKSDSDK

SEQ ID NO: 21 - protein, 9 aa, synthetic construct
MIWHSSAAV

SEQ ID NO: 22 - protein, 128 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGS

SEQ ID NO: 23 - protein, 116 aa, synthetic construct
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 24 - protein, 42 aa, synthetic construct
KRGRKKLLYIFKQPFMRPVQTTQEEDGCSCRFPEEEEGGCEL

SEQ ID NO: 25 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 46 aa, synthetic construct
PTTTPAPRPPTPAPTIASQPLSLRPEACRPAAGGAVHTRGLDFACD

SEQ ID NO: 27 - protein, 25 aa, synthetic construct
IYIWAPLAGTCGVLLLSLVITLYCN

SEQ ID NO: 28 - protein, 400 aa, synthetic construct
MYRMQLLSCIALSLALVTNSATSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 29 - protein, 235 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRSGGGSGGGGSGGGGSGGGGSGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 15 aa, synthetic construct
GGGGSGGGGSGGGGS

SEQ ID NO: 31 - protein, 355 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRSGGGSGGGGSGGGGSGGGGSGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGATNFSLLKQAGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 32 - protein, 26 aa, synthetic construct
SGGGSGGGGSGGGGSGGGGSGGGSLQ

SEQ ID NO: 33 - protein, 20 aa, synthetic construct
SGGSGGGGSGGGSGGGGSLQ

SEQ ID NO: 34 - protein, 929 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSATSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 35 - protein, 764 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAPRRARGCRTLGLPALLLLLLLRPPATRGITCPPPMSVEHADIWVKSYSLYSRERYICNSGFKRKAGTSSLTECVLNKATNVAHWTTPSLKCIRSGGGSGGGGSGGGGSGGGGSGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 36 - protein, 1049 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSATSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGSGATNFSLLKQAGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 37 - DNA, 2787 bp, synthetic construct
  source 1..2787: mol_type = other DNA
atggactggacatggcggatactcttcctcgtcgctgctgcaaccggagcccacagccaggtgcagctccagcagtctgggccaggtttggtgactcctagtcagacattgagcttgacttgtgctatcagcggagactctgtttcatctaattctgcaacttggaactggattcggcagtcccccagccgggggctcgagtggttgggtcggacctactatcggagcaaatggtacaatgactatgcagtgagcgtcaaatcaagaatgagcatcaatcctgacacaagcaagaaccagtttagccttcagcttaatagcgtgactccagaggacacagctgtgtactattgcgcgagaggcatgatgacatactattacggaatggacgtgtggggccagggaactactgttacagtgtcaagcggaatcctcggtagcggaggcggcggttccggcggagggggtagtggtggcgggggtagtcaacctgtgctgacccagagcagctctcttagtgctagcccaggtgcaagtgcaagtcttacctgtacactgcgctccggtattaatgtgggcccttaccgaatttactggtaccagcagaaaccaggctcccctccccagtatctgctgaactataagtctgactcagacaaacagcagggctccggtgtgccatcccgatttagtggctcaaaggatgctagtgcaaatgccggtgttctcctgatcagcggactcagatcagaggacgaagcagactattactgtatgatttggcatagcagcgctgctgtcttcggaggagggactcagctcactgtcttgagtcgggccgctgcacctaccactacccctgcccctcgaccccctactcccgccccaactatcgcatcccaaccactctctctcagacccgaagcctgtagacccgcagccggtggcgctgtgcatactcgcggacttgattttgcttgtgatatttatatctgggccccccttgccggaacttgtggagttctcctgctgtctctcgtaatcaccctttattgtaacaaacgggggcgcaaaaaacttctttacattttcaagcagccctttatgcggcccgtgcagaccacacaggaagaagatggctgcagctgcaggttcccagaggaagaagagggcggctgcgagctgcgagtaaagttcagccggagcgccgatgcacctgcataccagcagggtcagaaccagctctacaatgagctgaacctgggcagaagagaggaatatgatgtactcgacaagagaaggggacgcgatccagagatgggcggcaaaccacggcggaaaaatccgcaggaggggctctataacgagctccagaaggacaagatggcagaagcctactcagaaattggcatgaaaggagagagaaggaggggaaagggccatgatggcctttaccaagggttgtctactgccaccaaggatacgtacgatgcactccatatgcaggctcttcctccccgaggttcaggcgcaacaaatttttcacttcttaaacaagctggcgatgtcgaggaaaacccaggtcccatgtatagaatgcagcttctgtcatgtatcgcactgagtctggccctggtgaccaacagtgccaccagcaactgggtgaatgtgataagcgaccttaagaaaatagaagaccttattcagtccatgcacatagatgccacactgtacaccgagagcgatgtgcacccttcctgcaaagtgacagctatgaaatgcttccttctggaactgcaagtaatttcattggaatctggcgatgcttccatacatgacaccgtggaaaaccttattattttggctaacaattcattgagctcaaatggaaacgtgacagaatccggttgtaaggaatgtgaagagctggaagaaaaaaatatcaaggaattcctgcagagctttgttcacattgtgcaaatgtttattaatacatcctcagggggcggttccggaggcgggggaagtggcggaggaggaagcggcggaggaggaagcggaggaggatcacttcaaatcacatgtcccccccctatgagtgttgaacatgctgacatctgggtgaaatcctattccctttattcaagagaacgatacatatgtaattccgggtttaagaggaaagcaggcacatcatctctcaccgaatgtgtcctgaataaggcgacaaacgtagctcactggactacgccctccctcaaatgcattagagacccagcactcgtgcaccaaaggccagcccccccaagcaccgtcactactgcaggtgtaaccccgcaaccagaatccctctcaccaagcggaaaagagccagccgcatcttctcctagttccaataatacagccgcgacaacagccgcaattgtccctggaagccagttgatgccatcaaagtccccaagtacgggtacgaccgaaatctcctcccacgaaagcagccacggaacaccaagccagactaccgccaagaactgggagctgaccgcttctgcatcacatcagccgccgggagtgtatccacaggggcactctgataccacagtagcaatctcaacctccaccgtcctgctgtgtggccttagcgctgtgtctctcctcgcatgttacctcaaatccaggcagacccccccccttgctagtgtcgaaatggaggcaatggaagcacttcccgtgacatggggcacttctagcagagatgaggaccttgaaaactgctcacaccacctc

SEQ ID NO: 38 - DNA, 2292 bp, synthetic construct
  source 1..2292: mol_type = other DNA
atggactggacatggcggatactcttcctcgtcgctgctgcaaccggagcccacagccaggtgcagctccagcagtctgggccaggtttggtgactcctagtcagacattgagcttgacttgtgctatcagcggagactctgtttcatctaattctgcaacttggaactggattcggcagtcccccagccgggggctcgagtggttgggtcggacctactatcggagcaaatggtacaatgactatgcagtgagcgtcaaatcaagaatgagcatcaatcctgacacaagcaagaaccagtttagccttcagcttaatagcgtgactccagaggacacagctgtgtactattgcgcgagaggcatgatgacatactattacggaatggacgtgtggggccagggaactactgttacagtgtcaagcggaatcctcggtagcggaggcggcggttccggcggagggggtagtggtggcgggggtagtcaacctgtgctgacccagagcagctctcttagtgctagcccaggtgcaagtgcaagtcttacctgtacactgcgctccggtattaatgtgggcccttaccgaatttactggtaccagcagaaaccaggatcccctccccagtatctgctgaactataagtctgactcagacaaacagcagggctccggtgtgccatcccgatttagtggctcaaaggatgctagtgcaaatgccggtgttctcctgatcagcggactcagatcagaggacgaagcagactattactgtatgatttggcatagcagcgctgctgtcttcggaggagggactcagctcactgtcttgagtcgggccgctgcacctaccactacccctgcccctcgaccccctactcccgccccaactatcgcatcccaaccactctctctcagacccgaagcctgtagacccgcagccggtggcgctgtgcatactcgcggacttgattttgcttgtgatatttatatctgggccccccttgccggaacttgtggagttctcctgctgtctctcgtaatcaccctttattgtaacaaacgggggcgcaaaaaacttctttacattttcaagcagccctttatgcggcccgtgcagaccacacaggaagaagatggctgcagctgcaggttcccagaggaagaagagggcggctgcgagctgcgagtaaagttcagccggagcgccgatgcacctgcataccagcagggtcagaaccagctctacaatgagctgaacctgggcagaagagaggaatatgatgtactcgacaagagaaggggacgcgatccagagatgggcggcaaaccacggcggaaaaatccgcaggaggggctctataacgagctccagaaggacaagatggcagaagcctactcagaaattggcatgaaaggagagagaaggaggggaaagggccatgatggcctttaccaagggttgtctactgccaccaaggatacgtacgatgcactccatatgcaggctcttcctccccgagggtctggcgctacgaatttctctctccttaaacaggccggagacgtggaagaaaatcccggcccgatggcgccccgcagggcccgcgggtgtcgaacattgggtctgcctgctctcttgctgctgttgcttctcagacctcccgccacacgcggaattacgtgccctccccccatgtctgtggaacatgccgacatatgggtcaagtcttacagtctttactctagagaacggtatatctgcaatagcgggttcaaaagaaaagcagggacttccagcctgacagagtgcgtactgaataaggccactaacgttgctcactggaccaccccatcattgaagtgtattcgatcaggaggcggaagcggtggtgggggctcagggggtggcggtagtggaggcgggggcagcggagggggctctttgcaaaactgggttaatgttattagcgaccttaagaaaatcgaggacctgatacagtccatgcacatcgatgcgaccctgtacactgagagcgatgtgcatcccagttgcaaagtgactgctatgaaatgctttctgctcgagttgcaggtgatctccctggaaagcggcgacgcctcaatacacgacacggtcgaaaatctgatcattctcgccaacaactctctctcaagtaacgggaatgtgacagaaagtggatgcaaagaatgcgaggaacttgaggagaaaaacattaaagaattcctccagtccttcgtccacatcgtgcagatgtttatcaatacttcc

SEQ ID NO: 39 - DNA, 3147 bp, synthetic construct
  source 1..3147: mol_type = other DNA
atggattggacctggcgaatactcttcctcgtcgcagcggccactggtgcccattcacaagtccaactgcagcagagcggacctggcctggtgacacccagtcagactctcagcctgacttgtgcaatcagcggcgatagtgtgtctagtaattctgcaacatggaactggatcagacaatcaccaagtcggggactggagtggctcggtagaacctattataggtccaaatggtataacgattatgcagtgtccgtgaagtcccgaatgtctatcaaccctgatactagtaagaatcaattcagtctgcagcttaacagcgtaacccccgaagatactgctgtgtattactgtgcccggggtatgatgacttactactacggaatggatgtgtgggggcagggaacaaccgttactgtttcatccggcattctcgggagcggaggcggtggaagcggtgggggagggtccgggggaggaggatctcagcctgttcttactcaatcttcttccctctccgcctcacccggggcctccgcctcactgacctgcactctgcgatcaggcatcaacgttgggccttatagaatctactggtaccagcaaaagcctggatcaccgccccagtacctgctgaactataaatcagactcagacaagcagcagggctccggcgtgccgagtcgatttagcgggagcaaggacgcgtctgctaatgccggcgtgcttctcatcagcgggctccgcagtgaggatgaggcagattactactgcatgatttggcatagcagtgcagccgtatttggcggaggaacacagctgactgtcctctctcgcgccgccgctccgaccaccacccctgcaccacgcccacctactcctgcgccaaccattgccagccagcctctctctctccgacccgaggcctgtagacctgccgctggcggtgcagttcatactcggggtctcgatttcgcctgcgacatctacatctgggcaccactggctggcacttgtggcgttttgctcctgtccctggtgatcactctctactgtaataagagggggaggaagaaactcctgtatattttcaaacaaccctttatgcgccctgtccaaaccacccaggaagaagatggatgtagttgcagattcccagaagaagaggagggtgggtgtgaacttagggtgaagtttagtcgcagtgccgacgctcccgcttaccaacagggtcagaaccaactctacaatgagctgaatctggggaggcgcgaagaatacgacgttctggataaaagacgcggccgcgaccccgagatgggcgggaaaccgcggagaaagaacccacaggaaggattgtacaatgagctccagaaagataagatggcagaagcctactccgagatcggcatgaagggggagcgaaggcgcgggaaaggacacgatgggctgtaccagggtctttcaaccgcgacaaaggacacctatgatgctctccatatgcaggccctcccgccacgcggaagtggagcaactaattttagccttctgaaacaagctggcgatgttgaggaaaatcctgggccgatgtacaggatgcagctgctttcttgcattgcactgagtttggcactcgtcaccaactctgccacatcaaattgggttaacgttatcagcgatctgaagaaaatcgaggatttgatccagagtatgcatattgacgcaaccttgtatacagaatctgatgtgcacccaagctgtaaagtcacagctatgaaatgctttttgctggaactccaagtgatctccctcgaatccggcgatgcatccatccacgatactgtcgaaaaccttataattttggccaataacagcctcagcagcaatggcaacgtgacagagtctgggtgtaaggagtgtgaagaactggaggagaaaaatattaaagaattcctgcagtcctttgtacacattgtgcaaatgttcattaacacttcaagtggcggcgggagcggcgggggtggttcaggtggtggcggcagcggtggtggggggtctggcgggggtagtctccaaattacttgtcctcccccaatgagcgttgaacacgccgacatttgggtcaagtcttattcactgtacagccgagaaagatatatctgtaactctggatttaagcgcaaggccggaacgtctagtctgactgagtgcgtgctgaataaggccactaatgttgcccactggactacccccagcctgaagtgtattcgcgatcctgccttggtgcaccaacgacccgcgccacccagcacagtcactactgccggtgtgactccacagcccgagtctttgtccccgagcggaaaggagcccgccgcatcttcaccttcttcaaataacacggccgccacaaccgctgcaatcgtcccaggtagtcaactgatgccctctaaaagcccctctacggggacaactgagataagcagccacgagtctagtcacggcacaccaagccagactaccgccaaaaactgggagctgaccgcctctgcctcacaccaaccaccaggcgtgtatccccaggggcacagcgacaccactgtggcaatcagcaccagcacggtactgttgtgcggactctctgccgtcagtctgctggcctgctacctgaaatccagacagactccccccctggccagcgtggaaatggaagctatggaggctctgcccgtgacctgggggactagctccagagatgaagacttggagaactgcagtcaccatctcgggtccggagccacgaatttctctctcctcaaacaagctggggatgttgaggagaaccctgggccaatggccctcttgctcgcactgtccctcctggtcctgtggacatcacccgcccccaccctgtccggcacgaatgacgcagaagactgctgcctgtctgtcacgcagaaacccatccccggctatatagtgcggaacttccattacctgctgatcaaggacggatgtagggtgccagccgtcgtcttcaccaccctgcgagggcgccagctgtgcgctcctcctgaccagccctgggtggagcggatcattcaacgcttgcagcgcacctcagcaaaaatgaaaagaagaagtagt

SEQ ID NO: 40 - DNA, 2652 bp, synthetic construct
  source 1..2652: mol_type = other DNA
atggattggacctggcgaatactcttcctcgtcgcagcggccactggtgcccattcacaagtccaactgcagcagagcggacctggcctggtgacacccagtcagactctcagcctgacttgtgcaatcagcggcgatagtgtgtctagtaattctgcaacatggaactggatcagacaatcaccaagtcggggactggagtggctcggtagaacctattataggtccaaatggtataacgattatgcagtgtccgtgaagtcccgaatgtctatcaaccctgatactagtaagaatcaattcagtctgcagcttaacagcgtaacccccgaagatactgctgtgtattactgtgcccggggtatgatgacttactactacggaatggatgtgtgggggcagggaacaaccgttactgtttcatccggcattctcgggagcggaggcggtggaagcggtgggggagggtccgggggaggaggatctcagcctgttcttactcaatcttcttccctctccgcctcacccggggcctccgcctcactgacctgcactctgcgatcaggcatcaacgttgggccttatagaatctactggtaccagcaaaagcctggatcaccgccccagtacctgctgaactataaatcagactcagacaagcagcagggctccggcgtgccgagtcgatttagcgggagcaaggacgcgtctgctaatgccggcgtgcttctcatcagcgggctccgcagtgaggatgaggcagattactactgcatgatttggcatagcagtgcagccgtatttggcggaggaacacagctgactgtcctctctcgcgccgccgctccgaccaccacccctgcaccacgcccacctactcctgcgccaaccattgccagccagcctctctctctccgacccgaggcctgtagacctgccgctggcggtgcagttcatactcggggtctcgatttcgcctgcgacatctacatctgggcaccactggctggcacttgtggcgttttgctcctgtccctggtgatcactctctactgtaataagagggggaggaagaaactcctgtatattttcaaacaaccctttatgcgccctgtccaaaccacccaggaagaagatggatgtagttgcagattcccagaagaagaggagggtgggtgtgaacttagggtgaagtttagtcgcagtgccgacgctcccgcttaccaacagggtcagaaccaactctacaatgagctgaatctggggaggcgcgaagaatacgacgttctggataaaagacgcggccgcgaccccgagatgggcgggaaaccgcggagaaagaacccacaggaaggattgtacaatgagctccagaaagataagatggcagaagcctactccgagatcggcatgaagggggagcgaaggcgcgggaaaggacacgatgggctgtaccagggtctttcaaccgcgacaaaggacacctatgatgctctccatatgcaggccctcccgccacgcggaagtggagcaactaattttagccttctgaaacaagctggcgatgttgaggaaaatcctgggccgatggcacctagacgggcacgcgggtgtagaacgctgggcctccccgcactgttgttgctcttgcttctgagacctcccgctacaagggggataacttgccctccacctatgagcgtcgagcatgctgacatttgggtgaagtcctattcactctattcccgggagcggtacatctgtaactctggattcaagaggaaagccggcaccagcagtctgaccgagtgcgtgctgaataaggccaccaatgtggcccactggacaacccctagccttaaatgtatacggtcagggggcggatctggaggcggcggctccggtggaggcgggagtgggggcgggggctctggaggtggtagcctgcagaattgggttaacgtgattagcgacctcaaaaaaatcgaagatcttatccagagcatgcatatagacgcaaccctgtacacagaaagcgatgttcacccgtcctgcaaggtaacggctatgaagtgttttcttttggagttgcaagtcatatcactggaaagtggggatgcctcaattcacgataccgtggagaacctcatcatcctcgcaaataacagcctgagctccaatggcaatgtcacagagtcaggttgcaaagagtgtgaagagctggaagagaaaaacatcaaagagttcctccagtcatttgtgcacattgtccagatgttcattaacactagtggtagtggtgccacaaattttagtctgttgaaacaggccggggacgtcgaagaaaacccggggcctatggccctcttgctcgcactgtccctcctggtcctgtggacatcacccgcccccaccctgtccggcacgaatgacgcagaagactgctgcctgtctgtcacgcagaaacccatccccggctatatagtgcggaacttccattacctgctgatcaaggacggatgtagggtgccagccgtcgtcttcaccaccctgcgagggcgccagctgtgcgctcctcctgaccagccctgggtggagcggatcattcaacgcttgcagcgcacctcagcaaaaatgaaaagaagaagtagt

SEQ ID NO: 41 - protein, 17 aa, synthetic construct
GSGGGGSGGGGSGGGGS

SEQ ID NO: 42 - DNA, 1524 bp, synthetic construct
  source 1..1524: mol_type = other DNA
atggactggacatggcggatactcttcctcgtcgctgctgcaaccggagcccacagccaggtgcagctccagcagtctgggccaggtttggtgactcctagtcagacattgagcttgacttgtgctatcagcggagactctgtttcatctaattctgcaacttggaactggattcggcagtcccccagccgggggctcgagtggttgggtcggacctactatcggagcaaatggtacaatgactatgcagtgagcgtcaaatcaagaatgagcatcaatcctgacacaagcaagaaccagtttagccttcagcttaatagcgtgactccagaggacacagctgtgtactattgcgcgagaggcatgatgacatactattacggaatggacgtgtggggccagggaactactgttacagtgtcaagcggaatcctcggtagcggaggcggcggttccggcggagggggtagtggtggcgggggtagtcaacctgtgctgacccagagcagctctcttagtgctagcccaggtgcaagtgcaagtcttacctgtacactgcgctccggtattaatgtgggcccttaccgaatttactggtaccagcagaaaccaggctcccctccccagtatctgctgaactataagtctgactcagacaaacagcagggctccggtgtgccatcccgatttagtggctcaaaggatgctagtgcaaatgccggtgttctcctgatcagcggactcagatcagaggacgaagcagactattactgtatgatttggcatagcagcgctgctgtcttcggaggagggactcagctcactgtcttgagtcgggccgctgcacctaccactacccctgcccctcgaccccctactcccgccccaactatcgcatcccaaccactctctctcagacccgaagcctgtagacccgcagccggtggcgctgtgcatactcgcggacttgattttgcttgtgatatttatatctgggccccccttgccggaacttgtggagttctcctgctgtctctcgtaatcaccctttattgtaacaaacgggggcgcaaaaaacttctttacattttcaagcagccctttatgcggcccgtgcagaccacacaggaagaagatggctgcagctgcaggttcccagaggaagaagagggcggctgcgagctgcgagtaaagttcagccggagcgccgatgcacctgcataccagcagggtcagaaccagctctacaatgagctgaacctgggcagaagagaggaatatgatgtactcgacaagagaaggggacgcgatccagagatgggcggcaaaccacggcggaaaaatccgcaggaggggctctataacgagctccagaaggacaagatggcagaagcctactcagaaattggcatgaaaggagagagaaggaggggaaagggccatgatggcctttaccaagggttgtctactgccaccaaggatacgtacgatgcactccatatgcaggctcttcctccccgatga

SEQ ID NO: 43 - protein, 507 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 629 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGATNFSLLKQAGDVEENPGPMERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 45 - protein, 20 aa, synthetic construct
GILGSGGGGSGGGGSGGGGS

SEQ ID NO: 46 - protein, 507 aa, synthetic construct; identical to SEQ ID NO: 43

SEQ ID NO: 47 - protein, 29 aa, synthetic construct
MRISKPHLRSISIQCYLCLLLNSHFLTEA

SEQ ID NO: 48 - protein, 19 aa, synthetic construct
GIHVFILGCFSAGLPKTEA

SEQ ID NO: 49 - skipped

SEQ ID NO: 50 - DNA, 1521 bp, synthetic construct
  source 1..1521: mol_type = other DNA
atggactggacatggcggatactcttcctcgtcgctgctgcaaccggagcccacagccaggtgcagctccagcagtctgggccaggtttggtgactcctagtcagacattgagcttgacttgtgctatcagcggagactctgtttcatctaattctgcaacttggaactggattcggcagtcccccagccgggggctcgagtggttgggtcggacctactatcggagcaaatggtacaatgactatgcagtgagcgtcaaatcaagaatgagcatcaatcctgacacaagcaagaaccagtttagccttcagcttaatagcgtgactccagaggacacagctgtgtactattgcgcgagaggcatgatgacatactattacggaatggacgtgtggggccagggaactactgttacagtgtcaagcggaatcctcggtagcggaggcggcggttccggcggagggggtagtggtggcgggggtagtcaacctgtgctgacccagagcagctctcttagtgctagcccaggtgcaagtgcaagtcttacctgtacactgcgctccggtattaatgtgggcccttaccgaatttactggtaccagcagaaaccaggctcccctccccagtatctgctgaactataagtctgactcagacaaacagcagggctccggtgtgccatcccgatttagtggctcaaaggatgctagtgcaaatgccggtgttctcctgatcagcggactcagatcagaggacgaagcagactattactgtatgatttggcatagcagcgctgctgtcttcggaggagggactcagctcactgtcttgagtcgggccgctgcacctaccactacccctgcccctcgaccccctactcccgccccaactatcgcatcccaaccactctctctcagacccgaagcctgtagacccgcagccggtggcgctgtgcatactcgcggacttgattttgcttgtgatatttatatctgggccccccttgccggaacttgtggagttctcctgctgtctctcgtaatcaccctttattgtaacaaacgggggcgcaaaaaacttctttacattttcaagcagccctttatgcggcccgtgcagaccacacaggaagaagatggctgcagctgcaggttcccagaggaagaagagggcggctgcgagctgcgagtaaagttcagccggagcgccgatgcacctgcataccagcagggtcagaaccagctctacaatgagctgaacctgggcagaagagaggaatatgatgtactcgacaagagaaggggacgcgatccagagatgggcggcaaaccacggcggaaaaatccgcaggaggggctctataacgagctccagaaggacaagatggcagaagcctactcagaaattggcatgaaaggagagagaaggaggggaaagggccatgatggcctttaccaagggttgtctactgccaccaaggatacgtacgatgcactccatatgcaggctcttcctccccga

SEQ ID NO: 51 - DNA, 1521 bp, synthetic construct
  source 1..1521: mol_type = other DNA
atggactggacatggcggatactcttcctcgtcgctgctgcaaccggagcccacagccaggtgcagctccagcagtctgggccaggtttggtgactcctagtcagacattgagcttgacttgtgctatcagcggagactctgtttcatctaattctgcaacttggaactggattcggcagtcccccagccgggggctcgagtggttgggtcggacctactatcggagcaaatggtacaatgactatgcagtgagcgtcaaatcaagaatgagcatcaatcctgacacaagcaagaaccagtttagccttcagcttaatagcgtgactccagaggacacagctgtgtactattgcgcgagaggcatgatgacatactattacggaatggacgtgtggggccagggaactactgttacagtgtcaagcggaatcctcggtagcggaggcggcggttccggcggagggggtagtggtggcgggggtagtcaacctgtgctgacccagagcagctctcttagtgctagcccaggtgcaagtgcaagtcttacctgtacactgcgctccggtattaatgtgggcccttaccgaatttactggtaccagcagaaaccaggatcccctccccagtatctgctgaactataagtctgactcagacaaacagcagggctccggtgtgccatcccgatttagtggctcaaaggatgctagtgcaaatgccggtgttctcctgatcagcggactcagatcagaggacgaagcagactattactgtatgatttggcatagcagcgctgctgtcttcggaggagggactcagctcactgtcttgagtcgggccgctgcacctaccactacccctgcccctcgaccccctactcccgccccaactatcgcatcccaaccactctctctcagacccgaagcctgtagacccgcagccggtggcgctgtgcatactcgcggacttgattttgcttgtgatatttatatctgggccccccttgccggaacttgtggagttctcctgctgtctctcgtaatcaccctttattgtaacaaacgggggcgcaaaaaacttctttacattttcaagcagccctttatgcggcccgtgcagaccacacaggaagaagatggctgcagctgcaggttcccagaggaagaagagggcggctgcgagctgcgagtaaagttcagccggagcgccgatgcacctgcataccagcagggtcagaaccagctctacaatgagctgaacctgggcagaagagaggaatatgatgtactcgacaagagaaggggacgcgatccagagatgggcggcaaaccacggcggaaaaatccgcaggaggggctctataacgagctccagaaggacaagatggcagaagcctactcagaaattggcatgaaaggagagagaaggaggggaaagggccatgatggcctttaccaagggttgtctactgccaccaaggatacgtacgatgcactccatatgcaggctcttcctccccga

SEQ ID NO: 52 - DNA, 1521 bp, synthetic construct
  source 1..1521: mol_type = other DNA
atggattggacctggcgaatactcttcctcgtcgcagcggccactggtgcccattcacaagtccaactgcagcagagcggacctggcctggtgacacccagtcagactctcagcctgacttgtgcaatcagcggcgatagtgtgtctagtaattctgcaacatggaactggatcagacaatcaccaagtcggggactggagtggctcggtagaacctattataggtccaaatggtataacgattatgcagtgtccgtgaagtcccgaatgtctatcaaccctgatactagtaagaatcaattcagtctgcagcttaacagcgtaacccccgaagatactgctgtgtattactgtgcccggggtatgatgacttactactacggaatggatgtgtgggggcagggaacaaccgttactgtttcatccggcattctcgggagcggaggcggtggaagcggtgggggagggtccgggggaggaggatctcagcctgttcttactcaatcttcttccctctccgcctcacccggggcctccgcctcactgacctgcactctgcgatcaggcatcaacgttgggccttatagaatctactggtaccagcaaaagcctggatcaccgccccagtacctgctgaactataaatcagactcagacaagcagcagggctccggcgtgccgagtcgatttagcgggagcaaggacgcgtctgctaatgccggcgtgcttctcatcagcgggctccgcagtgaggatgaggcagattactactgcatgatttggcatagcagtgcagccgtatttggcggaggaacacagctgactgtcctctctcgcgccgccgctccgaccaccacccctgcaccacgcccacctactcctgcgccaaccattgccagccagcctctctctctccgacccgaggcctgtagacctgccgctggcggtgcagttcatactcggggtctcgatttcgcctgcgacatctacatctgggcaccactggctggcacttgtggcgttttgctcctgtccctggtgatcactctctactgtaataagagggggaggaagaaactcctgtatattttcaaacaaccctttatgcgccctgtccaaaccacccaggaagaagatggatgtagttgcagattcccagaagaagaggagggtgggtgtgaacttagggtgaagtttagtcgcagtgccgacgctcccgcttaccaacagggtcagaaccaactctacaatgagctgaatctggggaggcgcgaagaatacgacgttctggataaaagacgcggccgcgaccccgagatgggcgggaaaccgcggagaaagaacccacaggaaggattgtacaatgagctccagaaagataagatggcagaagcctactccgagatcggcatgaagggggagcgaaggcgcgggaaaggacacgatgggctgtaccagggtctttcaaccgcgacaaaggacacctatgatgctctccatatgcaggccctcccgccacgc

SEQ ID NO: 53 - protein, 954 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 54 - protein, 691 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 55 - protein, 811 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGATNFSLLKQAGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 56 - protein, 811 aa, synthetic construct; identical to SEQ ID NO: 55

SEQ ID NO: 57 - protein, 872 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAPRRARGCRTLGLPALLLLLLLRPPATRGITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGATNFSLLKQAGDVEENPGPMALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 58 - DNA, 2865 bp, synthetic construct
  source 1..2865: mol_type = other DNA
atggactggacatggcggatactcttcctcgtcgctgctgcaaccggagcccacagccaggtgcagctccagcagtctgggccaggtttggtgactcctagtcagacattgagcttgacttgtgctatcagcggagactctgtttcatctaattctgcaacttggaactggattcggcagtcccccagccgggggctcgagtggttgggtcggacctactatcggagcaaatggtacaatgactatgcagtgagcgtcaaatcaagaatgagcatcaatcctgacacaagcaagaaccagtttagccttcagcttaatagcgtgactccagaggacacagctgtgtactattgcgcgagaggcatgatgacatactattacggaatggacgtgtggggccagggaactactgttacagtgtcaagcggaatcctcggtagcggaggcggcggttccggcggagggggtagtggtggcgggggtagtcaacctgtgctgacccagagcagctctcttagtgctagcccaggtgcaagtgcaagtcttacctgtacactgcgctccggtattaatgtgggcccttaccgaatttactggtaccagcagaaaccaggctcccctccccagtatctgctgaactataagtctgactcagacaaacagcagggctccggtgtgccatcccgatttagtggctcaaaggatgctagtgcaaatgccggtgttctcctgatcagcggactcagatcagaggacgaagcagactattactgtatgatttggcatagcagcgctgctgtcttcggaggagggactcagctcactgtcttgagtcgggccgctgcacctaccactacccctgcccctcgaccccctactcccgccccaactatcgcatcccaaccactctctctcagacccgaagcctgtagacccgcagccggtggcgctgtgcatactcgcggacttgattttgcttgtgatatttatatctgggccccccttgccggaacttgtggagttctcctgctgtctctcgtaatcaccctttattgtaacaaacgggggcgcaaaaaacttctttacattttcaagcagccctttatgcggcccgtgcagaccacacaggaagaagatggctgcagctgcaggttcccagaggaagaagagggcggctgcgagctgcgagtaaagttcagccggagcgccgatgcacctgcataccagcagggtcagaaccagctctacaatgagctgaacctgggcagaagagaggaatatgatgtactcgacaagagaaggggacgcgatccagagatgggcggcaaaccacggcggaaaaatccgcaggaggggctctataacgagctccagaaggacaagatggcagaagcctactcagaaattggcatgaaaggagagagaaggaggggaaagggccatgatggcctttaccaagggttgtctactgccaccaaggatacgtacgatgcactccatatgcaggctcttcctccccgaggctcaggagccaccaacttctccctgctgaagcaggccggcgacgtggaggagaacccaggtcctatgagaatctcaaaaccccatcttagaagcatctctatacagtgttatctgtgtctcttgctgaactcccactttttgacagaagctgggatacatgtctttatcctgggatgtttttccgccgggctccctaaaaccgaggccaactgggtaaacgtaatctcagaccttaaaaagattgaggacctgattcagtcaatgcatatcgatgcaactttgtacacggagagcgatgttcacccaagttgtaaagtgaccgcgatgaaatgttttctcctcgaattgcaggtgatctccctcgagtcaggcgacgcgtctatccacgatactgtggaaaaccttatcattttggcgaacaatagcctctcatctaatggtaacgtgaccgagtccggctgcaaggaatgtgaggaactggaggagaaaaatatcaaggaattcctgcagtcatttgtacacatcgtgcaaatgtttatcaacacttcttcaggaggcgggtcaggagggggaggctcaggcggcggaggtagtggaggaggaggaagtggaggcggcagtctccagatcacctgtccaccaccaatgagtgtggaacacgcggacatttgggtcaagtcatattctctttactccagagagcgatacatatgcaacagtggtttcaagcggaaagcgggtacttcttcacttaccgagtgcgtgctcaataaagcaaccaacgtcgcgcactggacaacacctagcctgaaatgcataagagatcctgccctggttcaccagcggccagcgccaccgtccacagtgacaacagctggtgtgacaccccagccggagagccttagccctagcggcaaagagccggccgcaagctcaccaagctcaaataacacagccgcgacaactgctgctatcgtgcccggttcacaattgatgccgagcaaatcaccaagcaccggaactaccgaaatctcaagtcatgaaagtagtcacggtactcctagccagacgacggcaaagaattgggagctgactgcctctgcgagccaccagccgccgggtgtttaccctcaggggcattcagatactactgtggctatctctacttccaccgtcctcttgtgcggcttgtctgctgtgtctcttctggcttgctatttgaaaagtagacagacaccaccccttgcaagtgtcgagatggaagcgatggaggcattgcctgtgacctggggaaccagtagtagggacgaggacctggaaaattgtagtcaccacctgtga

SEQ ID NO: 59 - DNA, 2076 bp, synthetic construct
  source 1..2076: mol_type = other DNA
atggactggacatggcggatactcttcctcgtcgctgctgcaaccggagcccacagccaggtgcagctccagcagtctgggccaggtttggtgactcctagtcagacattgagcttgacttgtgctatcagcggagactctgtttcatctaattctgcaacttggaactggattcggcagtcccccagccgggggctcgagtggttgggtcggacctactatcggagcaaatggtacaatgactatgcagtgagcgtcaaatcaagaatgagcatcaatcctgacacaagcaagaaccagtttagccttcagcttaatagcgtgactccagaggacacagctgtgtactattgcgcgagaggcatgatgacatactattacggaatggacgtgtggggccagggaactactgttacagtgtcaagcggaatcctcggtagcggaggcggcggttccggcggagggggtagtggtggcgggggtagtcaacctgtgctgacccagagcagctctcttagtgctagcccaggtgcaagtgcaagtcttacctgtacactgcgctccggtattaatgtgggcccttaccgaatttactggtaccagcagaaaccaggctcccctccccagtatctgctgaactataagtctgactcagacaaacagcagggctccggtgtgccatcccgatttagtggctcaaaggatgctagtgcaaatgccggtgttctcctgatcagcggactcagatcagaggacgaagcagactattactgtatgatttggcatagcagcgctgctgtcttcggaggagggactcagctcactgtcttgagtcgggccgctgcacctaccactacccctgcccctcgaccccctactcccgccccaactatcgcatcccaaccactctctctcagacccgaagcctgtagacccgcagccggtggcgctgtgcatactcgcggacttgattttgcttgtgatatttatatctgggccccccttgccggaacttgtggagttctcctgctgtctctcgtaatcaccctttattgtaacaaacgggggcgcaaaaaacttctttacattttcaagcagccctttatgcggcccgtgcagaccacacaggaagaagatggctgcagctgcaggttcccagaggaagaagagggcggctgcgagctgcgagtaaagttcagccggagcgccgatgcacctgcataccagcagggtcagaaccagctctacaatgagctgaacctgggcagaagagaggaatatgatgtactcgacaagagaaggggacgcgatccagagatgggcggcaaaccacggcggaaaaatccgcaggaggggctctataacgagctccagaaggacaagatggcagaagcctactcagaaattggcatgaaaggagagagaaggaggggaaagggccatgatggcctttaccaagggttgtctactgccaccaaggatacgtacgatgcactccatatgcaggctcttcctccccgaggttcaggcgcaacaaatttttcacttcttaaacaagctggcgatgtcgaggaaaacccaggtcccatgcggatctctaaaccccacttgcggagcatttctatccagtgttatctttgcctcctgcttaactcccactttctcacagaagcagggatacacgtgttcatcctgggctgtttttctgccggtctccccaaaacagaagccaactgggtgaatgtgatcagtgatcttaagaaaatagaagacctcatccagtcaatgcacatcgatgccaccttgtacactgagagcgacgtgcacccttcctgcaaggtgacagctatgaagtgcttcctgcttgagctccaggtcatatcccttgagtctggagatgcaagtatccacgatacggtggaaaaccttattatactggccaataattctctttcttccaatggcaatgttaccgaatcagggtgtaaagagtgcgaagagctggaggagaaaaatatcaaagagtttttgcagtcatttgtgcacatcgtccagatgtttattaatacaagttga

SEQ ID NO: 60 - DNA, 3225 bp, synthetic construct
  source 1..3225: mol_type = other DNA
atggattggacctggcgaatactcttcctcgtcgcagcggccactggtgcccattcacaagtccaactgcagcagagcggacctggcctggtgacacccagtcagactctcagcctgacttgtgcaatcagcggcgatagtgtgtctagtaattctgcaacatggaactggatcagacaatcaccaagtcggggactggagtggctcggtagaacctattataggtccaaatggtataacgattatgcagtgtccgtgaagtcccgaatgtctatcaaccctgatactagtaagaatcaattcagtctgcagcttaacagcgtaacccccgaagatactgctgtgtattactgtgcccggggtatgatgacttactactacggaatggatgtgtgggggcagggaacaaccgttactgtttcatccggcattctcgggagcggaggcggtggaagcggtgggggagggtccgggggaggaggatctcagcctgttcttactcaatcttcttccctctccgcctcacccggggcctccgcctcactgacctgcactctgcgatcaggcatcaacgttgggccttatagaatctactggtaccagcaaaagcctggatcaccgccccagtacctgctgaactataaatcagactcagacaagcagcagggctccggcgtgccgagtcgatttagcgggagcaaggacgcgtctgctaatgccggcgtgcttctcatcagcgggctccgcagtgaggatgaggcagattactactgcatgatttggcatagcagtgcagccgtatttggcggaggaacacagctgactgtcctctctcgcgccgccgctccgaccaccacccctgcaccacgcccacctactcctgcgccaaccattgccagccagcctctctctctccgacccgaggcctgtagacctgccgctggcggtgcagttcatactcggggtctcgatttcgcctgcgacatctacatctgggcaccactggctggcacttgtggcgttttgctcctgtccctggtgatcactctctactgtaataagagggggaggaagaaactcctgtatattttcaaacaaccctttatgcgccctgtccaaaccacccaggaagaagatggatgtagttgcagattcccagaagaagaggagggtgggtgtgaacttagggtgaagtttagtcgcagtgccgacgctcccgcttaccaacagggtcagaaccaactctacaatgagctgaatctggggaggcgcgaagaatacgacgttctggataaaagacgcggccgcgaccccgagatgggcgggaaaccgcggagaaagaacccacaggaaggattgtacaatgagctccagaaagataagatggcagaagcctactccgagatcggcatgaagggggagcgaaggcgcgggaaaggacacgatgggctgtaccagggtctttcaaccgcgacaaaggacacctatgatgctctccatatgcaggccctcccgccacgcggaagtggagcaactaattttagccttctgaaacaagctggcgatgttgaggaaaatcctgggccgatgcgcattagcaagccacatctgaggagtatcagcatccagtgctacctttgcctgctgctcaactctcactttctgacagaagctggcatccacgtcttcatcctggggtgcttcagcgccggcttgccgaagaccgaagccaactgggtgaatgtgatctccgacctcaagaagatcgaggacctgatccagagtatgcatattgatgctacactttacaccgagtccgatgttcaccctagttgtaaggtgactgccatgaaatgtttcttgctggagcttcaggtaataagccttgagtctggggatgcaagcattcatgacacggttgagaatctcatcatcctggcaaataattcactgtcttcaaatggtaacgttacagagagcggctgtaaggagtgcgaagagcttgaagagaaaaacatcaaggaattcctccagagtttcgtgcacatcgtgcaaatgttcatcaacacgagctctggaggcggatcaggaggcggaggatcaggggggggagggtcaggcggagggggatctggtggaggcagccttcaaatcacatgcccgccacctatgtccgttgagcacgccgacatatgggtgaagtcatattcactgtatagtcgggagaggtacatttgtaattcaggtttcaagcgaaaagctgggacatcaagcctgacagaatgcgtacttaacaaggccacaaatgtcgcccattggaccactccgagtctgaagtgtatacgagatcccgcactggtgcaccagcgacctgctccccctagtacagtaacaaccgcgggcgttacgccccagcctgaatccctgagcccatctggcaaggagcctgcagctagctctccgagcagcaataatactgcagcgaccactgcagccatcgtccccggctcccagctcatgcctagtaaaagtccgtctacaggaacgaccgaaatctccagccacgagtctagtcacgggaccccgagtcagaccactgccaagaactgggagcttacggccagtgcctcccatcaacccccgggcgtctacccgcaaggccatagcgacaccacagtcgccattagcacatctactgtcctcttgtgcgggctctccgctgtgtccctcctggcctgttatctcaagagcagacagacccccccattggcaagcgttgagatggaggcaatggaggctctgcccgttacttgggggacttcttcacgcgacgaggatctggagaactgctcccaccacctgggaagtggtgccacaaatttcagcctgctcaagcaggccggggatgttgaagagaacccagggccgatggccctcttgctcgcactgtccctcctggtcctgtggacatcacccgcccccaccctgtccggcacgaatgacgcagaagactgctgcctgtctgtcacgcagaaacccatccccggctatatagtgcggaacttccattacctgctgatcaaggacggatgtagggtgccagccgtcgtcttcaccaccctgcgagggcgccagctgtgcgctcctcctgaccagccctgggtggagcggatcattcaacgcttgcagcgcacctcagcaaaaatgaaaagaagaagtagttga

SEQ ID NO: 61 - DNA, 2436 bp, synthetic construct
  source 1..2436: mol_type = other DNA
atggattggacctggcgaatactcttcctcgtcgcagcggccactggtgcccattcacaagtccaactgcagcagagcggacctggcctggtgacacccagtcagactctcagcctgacttgtgcaatcagcggcgatagtgtgtctagtaattctgcaacatggaactggatcagacaatcaccaagtcggggactggagtggctcggtagaacctattataggtccaaatggtataacgattatgcagtgtccgtgaagtcccgaatgtctatcaaccctgatactagtaagaatcaattcagtctgcagcttaacagcgtaacccccgaagatactgctgtgtattactgtgcccggggtatgatgacttactactacggaatggatgtgtgggggcagggaacaaccgttactgtttcatccggcattctcgggagcggaggcggtggaagcggtgggggagggtccgggggaggaggatctcagcctgttcttactcaatcttcttccctctccgcctcacccggggcctccgcctcactgacctgcactctgcgatcaggcatcaacgttgggccttatagaatctactggtaccagcaaaagcctggatcaccgccccagtacctgctgaactataaatcagactcagacaagcagcagggctccggcgtgccgagtcgatttagcgggagcaaggacgcgtctgctaatgccggcgtgcttctcatcagcgggctccgcagtgaggatgaggcagattactactgcatgatttggcatagcagtgcagccgtatttggcggaggaacacagctgactgtcctctctcgcgccgccgctccgaccaccacccctgcaccacgcccacctactcctgcgccaaccattgccagccagcctctctctctccgacccgaggcctgtagacctgccgctggcggtgcagttcatactcggggtctcgatttcgcctgcgacatctacatctgggcaccactggctggcacttgtggcgttttgctcctgtccctggtgatcactctctactgtaataagagggggaggaagaaactcctgtatattttcaaacaaccctttatgcgccctgtccaaaccacccaggaagaagatggatgtagttgcagattcccagaagaagaggagggtgggtgtgaacttagggtgaagtttagtcgcagtgccgacgctcccgcttaccaacagggtcagaaccaactctacaatgagctgaatctggggaggcgcgaagaatacgacgttctggataaaagacgcggccgcgaccccgagatgggcgggaaaccgcggagaaagaacccacaggaaggattgtacaatgagctccagaaagataagatggcagaagcctactccgagatcggcatgaagggggagcgaaggcgcgggaaaggacacgatgggctgtaccagggtctttcaaccgcgacaaaggacacctatgatgctctccatatgcaggccctcccgccacgcggaagtggagcaactaattttagccttctgaaacaagctggcgatgttgaggaaaatcctgggccgatgcgcatctccaagccccatctgaggagcatcagcatccagtgctacctgtgtctgctgctcaacagccacttcctgacggaagcaggcattcatgtctttatcctgggatgcttttctgccggcctgccaaagacagaagcaaactgggttaacgttatcagtgatctgaaaaaaatcgaggacctgatccagtccatgcatattgacgctacgctgtatacagagtccgacgtccacccatcatgcaaggtgaccgctatgaagtgtttcctgctggaactgcaggttatcagcttggaaagtggcgacgcttccattcacgatacggtggagaacttgataatccttgcgaataatagtctgagcagcaacggcaacgttactgaaagcgggtgcaaagaatgtgaagagctcgaagagaaaaacatcaaagaatttttgcagtctttcgtgcatattgttcagatgtttattaacaccagtggatcaggagcaactaacttctctcttcttaagcaagctggcgatgtagaggaaaaccctgggcctatggccctcttgctcgcactgtccctcctggtcctgtggacatcacccgcccccaccctgtccggcacgaatgacgcagaagactgctgcctgtctgtcacgcagaaacccatccccggctatatagtgcggaacttccattacctgctgatcaaggacggatgtagggtgccagccgtcgtcttcaccaccctgcgagggcgccagctgtgcgctcctcctgaccagccctgggtggagcggatcattcaacgcttgcagcgcacctcagcaaaaatgaaaagaagaagtagttga

SEQ ID NO: 62 - skipped

SEQ ID NO: 63 - protein, 24 aa, synthetic construct
IYIWAPLAGTCGVLLLSLVITLYC

SEQ ID NO: 64 - protein, 45 aa, synthetic construct
TTTPAPRPPTPAPTIASQPLSLRPEACRPAAGGAVHTRGLDFACD

SEQ ID NO: 65 - protein, 503 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 501 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 502 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 502 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 506 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 505 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAATTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 505 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 505 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 506 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 506 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAATTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 504 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 150 aa, synthetic construct
  VARIANT 1..150: note = This sequence may encompass 1-30 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 77 - protein, 19 aa, synthetic construct
ATNFSLLKQAGDVEENPGP

SEQ ID NO: 78 - protein, 20 aa, synthetic construct
QCTNYALLKLAGDVESNPGP

SEQ ID NO: 79 - protein, 22 aa, synthetic construct
VKQTLNFDLLKLAGDVESNPGP

SEQ ID NO: 80 - protein, 18 aa, synthetic construct
EGRGSLLTCGDVEENPGP

SEQ ID NO: 81 - DNA, 470 bp, synthetic construct
  source 1..470: mol_type = other DNA
ctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 82 - protein, 7 aa, synthetic construct
GILGSSG

(19) United States
(12) Patent Application Publication
(10) Pub. No.: US 2024/0374721 A1
(43) Pub. Date: Nov. 14, 2024

(54) CAR-EXPRESSING IMMUNE CELLS THAT SPECIFICALLY TARGET MESOTHELIN AND USES THEREOF

(71) Applicant: Takeda Pharmaceutical Company Limited, Chuo-ko, Osaka-shi, Osaka (JP)

(72) Inventors: Chihiro Take, Kanagawa (JP); Gary Shapiro, Cambridge, MA (US); Yoshiaki Kassai, Kanagawa (JP); Xingyue He, Cambridge, MA (US); Chantal Kuhn, Cambridge, MA (US)

(73) Assignee: Takeda Pharmaceutical Company Limited, Chuo-ko, Osaka-shi, Osaka (JP)

(21) Appl. No.: 18/292,574

(22) PCT Filed: Jul. 28, 2022
(86) PCT No.: PCT/US2022/038640

§ 371 Date: Jan. 26, 2024

Related U.S. Application Data
(60) Provisional application No. 63/227,115, filed on Jul. 29, 2021.
(60) Provisional application No. 63/306,862, filed on Feb. 4, 2022.

(51) Int. Cl. A61K 39/00; A61P 35/00; C07K 14/52; C07K 14/54; C07K 14/705; C07K 14/725; C07K 16/30; C12N 5/0783

(52) U.S. Cl. CPC A61K 39/4611; A61K 39/4631; A61K 39/4635; A61K 39/464468; A61P 35/00; C07K 14/521; C07K 14/5443; C07K 14/7051; C07K 14/70517; C07K 14/70578; C07K 16/30; C12N 5/0636; C07K 2317/53; C07K 2317/622; C07K 2317/73; C07K 2319/02; C07K 2319/03; C12N 2506/45; C12N 2510/00

(57) ABSTRACT

Disclosed herein are engineered immune cells that specifically recognizes mesothelin and expresses IL-15 and optionally CCL19. Also disclosed herein are isolated nucleic acid molecules comprising a polynucleotide encoding a chimeric antigen receptor (CAR) comprising an antibody that specifically recognizes mesothelin, and a 4-IBB intracellular region; and a polynucleotide encoding IL-15; and optionally a polynucleotide encoding CCL19, vectors, pharmaceutical compositions comprising the nucleic acid molecules, and methods of using the engineered immune cells.

### CROSS-REFERENCE TO RELATED APPLICATIONS

This application is the U.S. National Stage of PCT/US2022/038640, filed Jul. 28, 2022, which claims priority under 35 U.S.C § 119 (e) to U.S. Provisional Application No. 63/306,862, filed Feb. 4, 2022, and U.S. Provisional Application No. 63/227,115, filed Jul. 29, 2021, which are hereby incorporated by reference in their entireties.

The instant application contains a Sequence Listing which has been submitted in XML format and is hereby incorporated by reference in its entirety. Said ASCII copy, created on Jan. 23, 2024, is named 087147-0685_generic_SL.xml and is 117,713 bytes.

### TECHNICAL FIELD

The present invention relates to an immune cell that expresses a cell surface molecule specifically recognizing human mesothelin, interleukin 15 (IL-15), and optionally chemokine (C-C motif) ligand 19 (CCL19), a pharmaceutical composition comprising the immune cell, an expression vector comprising a polynucleotide encoding a cell surface molecule specifically recognizing mesothelin, a polynucleotide encoding IL-15, and optionally a polynucleotide encoding CCL19, a method of use, and a method for producing an immune cell that expresses a cell surface molecule specifically recognizing human mesothelin, IL-15, and optionally CCL19, comprising introducing a polynucleotide encoding the cell surface molecule specifically recognizing human mesothelin, a polynucleotide encoding the IL-15, and optionally a polynucleotide encoding the CCL19 to an immune cell.

### BACKGROUND OF THE DISCLOSURE

Malignant tumors are diseases that affect many people in the world and in general, are widely treated by chemotherapy, radiotherapy, or surgical therapy. However, there have been various problems such as the occurrence of adverse reactions, a loss of some functions, and recurrence or metastasis which cannot be treated. As such, the development of immune cell therapy has been advanced in recent years in order to maintain higher quality of life (QOL) of patients. Immune cell therapy involves harvesting immune cells from a patient, performing procedures to enhance the immune functions of the harvested immune cells, amplifying the cells, and readministering the cells back to the patient. For example, the immune cell therapy can include collecting T cells from a patient, introducing a nucleic acid encoding chimeric antigen receptor (constitutive androstane receptor: hereinafter, also referred to as “CAR”) to the T cells, and re-administering the T cells back to the patient. Although early success in blood cancers have been observed with CAR-T therapies; life-threatening toxicities and a substantial lack of efficacy in the treatment of solid tumors have also been observed. As such, improved CAR-T therapies are needed.

### PRIOR ART DOCUMENTS

### Patent Documents

Patent Document 1: WO2020/045610
Patent Document 2: US2020/0101142
Patent Document 3: WO2013/063419

### SUMMARY OF THE DISCLOSURE

### Objective Technical Problem to Be Solved by the Invention

There are a number of challenges that exist with immunotherapy with T cells, such as insufficient trafficking to solid tumors, high toxicity to normal tissues, the inability to overcome the immunosuppressive tumor microenvironment, and insufficient activation of the endogenous immune response. Further, immune cells modified to express CARs that specifically recognizes mesothelin have been shown to exhibit minimal therapeutic efficacy. Therefore, an objective technical problem to be solved is to provide optimized modified immune cells to target mesothelin-expressing cancers.

### Means to Solve the Objective Technical Problem

The present inventors have discovered that immune cells modified to express a CAR that specifically recognizes mesothelin, IL-15, and optionally CCL19 can improve therapeutic efficacy of the immunotherapy and to improve survival rate.

An isolated nucleic acid molecule comprising a first polynucleotide encoding a chimeric antigen receptor (CAR) comprising an antibody that specifically recognizes human mesothelin, and a 4-1BB intracellular region, and a second polynucleotide encoding an interleukin 15 (IL-15). In some embodiments, the CAR further comprises a CD8 hinge region, a CD8 transmembrane region and a and a CD3ζintracellular region. In some embodiments, the isolated nucleic acid molecule comprises the first polynucleotide encoding a CAR and the second polynucleotide encoding IL-15. In some embodiments, the isolated nucleic acid molecule further comprises a third polynucleotide encoding CCL19. In some embodiments, the IL-15 is human IL-15. In some embodiments, the CCL19 is human CCL19. In some embodiments, the antibody comprises a heavy chain variable region (V_{H}) and a light chain variable region (VL), wherein the VH comprises three complementarity-determining regions (CDRs) comprising SEQ ID NOs: 16-18, and wherein the VL comprises three CDRs comprising SEQ ID NOs: 19-21. In some embodiments, the VH comprises SEQ ID NO: 22 and the VL comprises SEQ ID NO: 23. In some embodiments, the antibody comprises a single-chain variable fragment (scFv) format. In some embodiments, the antibody comprises SEQ ID NO: 1. In some embodiments, the 4-1BB intracellular region comprises SEQ ID NO: 24. In some embodiments, the CD3ζintracellular region comprises SEQ ID NO: 25. In some embodiments, the 4-1BB intracellular region is upstream of the CD3ζintracellular region in the isolated nucleic acid molecule. In some embodiments, the CD8 hinge region comprises SEQ ID NO: 26. In some embodiments, the CD8 transmembrane region comprises SEQ ID NO: 27. In some embodiments, the CAR further comprises a peptide linker 3 to 10 amino acid residues in length linking the antibody and the CD8 hinge region. In some embodiments, the peptide linker comprises SEQ ID NO: 4. In some embodiments, the CAR molecule further comprises a signaling peptide. In some embodiments, the signaling peptide is located upstream of the antibody that specifically recognizes human mesothelin in the isolated nucleic acid molecule. In some embodiments, the signaling peptide comprises SEQ ID NO: 2. In some embodiments, the second polynucleotide encoding IL-15 and optionally the third polynucleotide encoding CCL19 are each independently transcribed under a promoter comprising a polynucleotide encoding a self-cleaving 2A peptide (2A peptide). In some embodiments, the 2A peptide comprises SEQ ID NO: 5. In some embodiments, the IL-15 comprises a sequence selected from SEQ ID NOs: 8-11. In some embodiments, the IL-15 comprises a sequence selected from SEQ ID NO: 28 or 29. In some embodiments, the CCL19 comprises SEQ ID NO: 13. In some embodiments, the first polynucleotide encoding the CAR and the second polynucleotide encoding IL-15 are arranged in the nucleic acid molecule from the 5′ terminus to the 3′ terminus as the first polynucleotide encoding the CAR—the second polynucleotide encoding IL-15. In some embodiments, the first polynucleotide encoding the CAR, the second polynucleotide encoding IL-15, and the third polynucleotide encoding CCL19 are arranged in the nucleic acid molecule from the 5′ terminus to the 3′ terminus as the first polynucleotide encoding the CAR—the second polynucleotide encoding IL-15—the third polynucleotide encoding CCL19. In some embodiments, the isolated nucleic acid molecule encodes a polypeptide comprising SEQ ID NO: 14 or 31. In some embodiments, the isolated nucleic acid molecule encodes a polypeptide comprising SEQ ID NO: 34, 35, 36, or 15. In some embodiments, the isolated nucleic acid molecule comprises SEQ ID NO: 37, 38, 39, or 40.

In certain embodiments, disclosed herein is a vector comprising the nucleic acid molecule described herein. In some embodiments, the vector is a viral vector, optionally an expression vector. In some embodiments, the viral vector is selected from a retrovirus vector, a lentivirus vector, an adenovirus vector, and an adeno-associated virus (AAV) vector. In some embodiments, the viral vector is a pSFG vector, a pMSGV vector or a pMSCV vector. In some embodiments, the vector is a plasmid.

In certain embodiments, disclosed herein is an immune cell comprising the nucleic acid molecule described herein or the vector described herein. In some embodiments, the immune cell further comprises a polynucleotide encoding gamma-TCR (γTCR) and a polynucleotide encoding delta-TCR (δTCR). In some embodiments, the γTCR is V gamma 9 TCR (Vγ9 TCR) and the δTCR is V delta 2 TCR (Vδ2TCR).

In certain embodiments, disclosed herein is an immune cell expressing: a) a chimeric antigen receptor (CAR) comprising an antibody that specifically recognizes human mesothelin, a CD8 hinge region, a CD8 transmembrane region, a 4-1BB intracellular region and a CD3ζ intracellular region; and b) IL-15; and c) optionally CCL19. In some embodiments, the immune cell is a T cell, a natural killer (NK) cell, a B cell, an antigen presenting cell, or a granulocyte, optionally a T cell or an NK cell. In some embodiments, the immune cell is derived from an induced pluripotent stem cell (iPSC). In some embodiments, the immune cell does not express alpha TCR (αTCR) and/or beta TCR (βTCR), optionally αβTCR. In some embodiments, the immune cell expresses a γδTCR. In some embodiments, the γδTCR comprises Vγ9 TCR and Vδ2TCR.

In certain embodiments, disclosed herein is a pharmaceutical composition comprising the immune cell described herein, and a pharmaceutically acceptable additive.

In certain embodiments, disclosed herein is a method of treating a mesothelin-expressing cancer comprising administering to a subject in need thereof the immune cell described herein or the pharmaceutical composition described herein. In some embodiments, the mesothelin-expressing cancer is a solid tumor, optionally selected from mesothelioma, colorectal cancer, pancreatic cancer, thymic cancer, bile duct cancer, lung cancer, skin cancer, breast cancer, prostate cancer, urinary bladder cancer, virginal cancer, neck cancer, uterine cancer, liver cancer, kidney cancer, spleen cancer, tracheal cancer, bronchial cancer, stomach cancer, esophageal cancer, gallbladder cancer, testis cancer, ovarian cancer, and bone cancer. In some embodiments, the mesothelin-expressing cancer is a hematopoietic cancer. In some embodiments, the mesothelin-expressing cancer is a sarcoma, optionally selected from chondrosarcoma, Ewing's sarcoma, malignant hemangioendothelioma, malignant schwannoma, osteosarcoma, and soft tissue sarcoma. In some embodiments, the mesothelin-expressing cancer is a metastatic cancer. In some embodiments, the mesothelin-expressing cancer is a relapsed cancer or a refractory cancer. In some embodiments, the method further comprises administering to the subject an additional therapeutic agent or an additional therapeutic regimen. In some embodiments, the additional therapeutic agent comprises a chemotherapeutic agent, an immunotherapeutic agent, a targeted therapy, radiation therapy, or a combination thereof. In some embodiments, the additional therapeutic regimen comprises a first-line therapy. In some embodiments, the additional therapeutic regimen comprises surgery. In some embodiments, the immune cell described herein or the pharmaceutical composition described herein and the additional therapeutic agent are administered simultaneously. In some embodiments, the immune cell described herein or the pharmaceutical composition described herein and the additional therapeutic agent are administered sequentially. In some embodiments, the immune cell described herein or the pharmaceutical composition described herein is administered to the subject prior to administration of the additional therapeutic agent. In some embodiments, the immune cell described herein or the pharmaceutical composition described herein is administered to the subject after administration of the additional therapeutic agent. In some embodiments, the subject is a human.

In certain embodiments, disclosed herein is a method of decreasing tumor cell proliferation comprising contacting the tumor cell with the immune cell described above, thereby decreasing the tumor cell proliferation. In some embodiments, the method is an in vitro method. In some embodiments, the method is an in vivo method.

In certain embodiments, disclosed herein is a method for producing an immune cell expressing cell surface molecules that specifically recognize human mesothelin, IL-15, and optionally CCL19, the method comprising: introducing a nucleic acid molecule described above or a vector described above to an immune cell to induce expression of cell surface molecules that specifically recognize human mesothelin, IL-15, and optionally CCL19 by the immune cell. In some embodiments, the immune cell is a T cell, a natural killer (NK) cell, a B cell, an antigen presenting cell, or a granulocyte, optionally a T cell or an NK cell.

In certain embodiments, disclosed herein is a kit comprising a nucleic acid molecule described above; a vector described above, an immune cell described above, or a pharmaceutical composition described above, and instructions of use.

### Effect of the Invention

The immune cell of the present invention has cytotoxic activity against cancer cells expressing mesothelin (e.g., human mesothelin) and is capable of suppressing the formation of tumor expressing mesothelin (e.g., human mesothelin). Also, the immune cell of the present invention has suppressive effects on the recurrence of cancer cells.

### BRIEF DESCRIPTION OF THE DRAWINGS

FIG. 1A shows a cartoon representation of an exemplary vector comprising a polynucleotide encoding a chimeric antigen receptor (CAR) that specifically recognizes mesothelin and a polynucleotide encoding IL-15.

FIG. 1B shows a cartoon representation of an exemplary vector comprising a polynucleotide encoding a chimeric antigen receptor (CAR) that specifically recognizes mesothelin, a polynucleotide encoding IL-15, and a polynucleotide encoding CCL19.

FIG. 1C shows a cartoon representation of a modified immune cell expressing a CAR that specifically recognizes mesothelin and IL-15.

FIG. 1D shows a cartoon representation of a modified immune cell expressing a CAR that specifically recognizes mesothelin, IL-15, and CCL19.

FIG. 2 shows the results obtained by staining the acquired cells by using an antibody set (Vδ1 Myltenyi FITC, Vδ2 Miltenyi APC, γδTCR BD BV510, CD3 BioLegend APC/Cy7 and αβTCR eBioscience FITC). The filled peaks show the results of the non-staining group, and the blank peaks show the staining results using each antigen-specific antibody.

FIG. 3 shows the results of flow cytometry obtained by staining the acquired cells by using an antibody set (Vδ1 Miltenyi FITC, Vδ2 Miltenyi APC, γδTCR BD BV510, CD3 BioLegend APC/Cy7 and αβTCR eBioscience FITC).

FIG. 4 shows the measurement results of cell proliferation of iPS cell-derived γδT cells (iγδT cells). The vertical axis shows the cell proliferation rate, and the horizontal axis shows the number of days elapsed from the day when stimulation with anti-CD3 antibody (UCHT1) and anti-CD30 antibody was started.

FIG. 5 shows the expression of CD3 and γδTCR molecules on the cell membrane surface of γδT cells (iγ9δ2T cells) differentiated by introducing Vγ9Vδ2TCR gene into iPS cells.

FIG. 6 shows the measurement results of cell proliferation of iMesothelin-CAR/IL-15γ982T cells. The vertical axis shows the number of cells, and the horizontal axis shows the number of days elapsed from the day when stimulation with anti-CD3 antibody (UCHT1) was started.

FIG. 7A shows mesothelin staining on tumor cell lines.

FIG. 7B shows iMeso-CAR/IL-15γ9δ2T cells kill mesothelin positive cell lines in vitro in a dose dependent manner.

FIG. 8 shows that iMeso-CAR/IL-15γδT cells comprising costimulatory domains CD28 or 4-1BB outperform other tested costimulatory domains.

FIG. 9A-FIG. 9D shows that iMeso-CAR/IL-15γ9δ2T cells with mIL15/Ra or sushi15 exhibits higher anti-tumor efficacy than other tested iMeso-CAR/IL-15γ9δ2T cells. FIG. 9A and FIG. 9B show Arm 1 regimen (5×10⁶ cells). FIG. 9C and FIG. 9D show Arm 2 regimen (1.5×10⁶ cells).

FIG. 9E shows that iMeso-CAR/IL-15γ9δ2T cells comprising 4-1BB exhibits higher anti-tumor effect than iMeso-CAR/IL-15γ9δ2T cells comprising CD28.

FIG. 10 shows GSU s.c. xenograft with intratumoral iMeso-CAR/IL-15γ9δ2T cells injection. iMeso-CAR/IL-15γ9δ2T cells expressing either mIL15/Ra or sushi15 exhibit higher efficacy than other tested iMeso-CAR/IL-15γ9δ2T cells.

FIG. 11 shows efficacy of i.p, and i.v, administered iMeso-CAR/IL-15γ9δ2T cells in the intraperitoneal GSU-redLuc xenograft model.

FIG. 12A shows CAR-T cells counted by flow cytometry and FACS phenotyping performed at indicated time-points.

FIG. 12B shows percent cytotoxicity in anti-Msln CAR-T cells, co-expressing a CAR against Msln together with a TGFβ modulator (e.g., TGFβR2-VH or dnTGFbR2) or a control VH against GFP (Msln-control V_{H}).

### DETAILED DESCRIPTION OF THE DISCLOSURE

### Engineered Immune Cells

In certain embodiments, disclosed herein are engineered immune cells that express an engineered cell surface molecule that specifically binds to mesothelin, interleukin 15 (IL-15), and optionally chemokine (C-C motif) ligand 19 (CCL19). In some embodiments, the engineered cell surface molecule comprises a chimeric antigen receptor (CAR) that specifically recognizes mesothelin or a T cell receptor (TCR) that specifically binds to mesothelin.

In some embodiments, the engineered immune cell contains an exogenous nucleic acid encoding the engineered cell surface molecule, an exogenous nucleic acid encoding IL-15, and optionally an exogenous nucleic acid encoding CCL19. In some embodiments, the engineered immune cell expresses a surface molecule that specifically recognizes mesothelin, IL-15, and optionally CCL19.

Mesothelin (MSLN) is a cell surface-bound glycosylphosphatidylinositol (GPI) anchored protein, in which normal expression is restricted to the mesothelial cells such as from the pleura, pericardium, peritoneum, tunica vaginalis, ovaries, or fallopian tubes. However, MSLN has also been shown to be overexpressed in a plethora of cancers, such as malignant mesothelioma, ovarian cancer, breast cancer (e.g., triple-negative breast cancer, TNBC), pancreatic cancer, lung cancer, gastric cancer, endometrial cancer, cervical cancer, biliary cancer, uterine serous carcinoma, cholangiocarcinoma, and pediatric acute myeloid leukemia. Further, increased MSLN expression has been associated with a poorer prognosis in patients with TNBC, ovarian cancer, lung adenocarcinoma, cholangiocarcinoma, and pancreatic adenocarcinoma.

The physiological and biological functions of MSLN have not been fully elucidated. However, MSLN has been shown to be involved in several mechanism of cancer pathogenesis. For example, in epithelial ovarian carcinoma, patients who exhibited higher levels of MSLN mRNA expression in surgery-resected ovarian cancer tissues showed resistance to chemotherapy with platinum and cyclophosphamide when compared with chemo-sensitive patients who expressed lower MSLN levels (Tang, et al., “The role of mesothelin in tumor progression and targeted therapy,” Anticancer Agents Med Chem. 13 (2): 276-280 (2013)). MSLN has also been found to bind with high affinity to the surface mucin MUC16 (or CA125) and the binding has been suggested to mediate adhesion of ovarian cancer cells to the mesothelial cells and promote metastasis (Rump, et al., “Binding of ovarian cancer antigen CA125/MUC16 to mesothelin mediates cell adhesion,” J Biol Chem 279 (10): 9190-9198, 2004; Gubbels, et al., “Mesothelin-MUC16 binding is a high affinity, N-glycan dependent interaction that facilitates peritoneal metastasis of ovarian tumors,” Mol Cancer 5 (1): 50, 2006). Further, MSLN has been shown to be involved in tumor progression, cell survival and proliferation in pancreatic cancer both in vitro and in vivo (Li, et al., “Mesothelin is a malignant factor and therapeutic vaccine target for pancreatic cancer,” Mol Cancer Ther. 7 (2): 286-296, 2008).

### Chimeric Antigen Receptors (CARs)

### A. Anti-Mesothelin Antibodies

In some embodiments, the engineered cell surface molecule comprises a chimeric antigen receptor (CAR) comprising an antibody that specifically recognizes mesothelin. In some embodiments, the antibody specifically recognizes a mammalian mesothelin, e.g., a rodent mesothelin, a non-human primate mesothelin, or a human mesothelin.

Human mesothelin, a 40 kDa protein, is encoded by the MSLN gene. Sequence information on human mesothelin can be appropriately obtained by the search of a publicly known document or a database such as NCBI (www.ncbi.nlm.nih.gov/guide/). Examples of the amino acid sequence information on human mesothelin can include GenBank accession No. NP_037536.2, AAV87530.1, and their isoforms.

In some embodiments, the anti-mesothelin antibody comprises a heavy chain variable region (V_{H}) comprising or consisting of CDRH1 as set forth in SEQ ID NO: 16, CDRH2 as set forth in SEQ ID NO: 17, and CDRH3 as set forth in SEQ ID NO: 18; and a light chain variable region (VL) comprising or consisting of CDRL1 as set forth in SEQ ID NO: 19, CDRL2 as set forth in SEQ ID NO: 20, and CDRL3 as set forth in SEQ ID NO: 21. See Table 1.

TABLE 1

| | | SEQ |
|---|---|---|
| | | ID |
| P4* | SEQUENCES | NO: |
| HCDR | GDSVSSNSAT | 16 |
| 1 | | |
| HCDR | TYYRSKWYN | 17 |
| 2 | | |
| HCDR | ARGMMTYYYGMDV | 18 |
| 3 | | |
| LCDR | SGINVGPYR | 19 |
| 1 | | |
| LCDR | YKSDSDK | 20 |
| 2 | | |
| LCDR | MIWHSSAAV | 21 |
| 3 | | |
| VH | QVQLQQSGPGLVTPSQTLSLTCAISGDSVSSNSATWNWIRQSP | 22 |
| | SRGLEWLGRTYYRSKWYNDYAVSVKSRMSINPDTSKNQFSL | |
| | QLNSVTPEDTAVYYCARGMMTYYYGMDVWGQGTTVTVSSG | |
| | ILGS | |
| VL | QPVLTQSSSLSASPGASASLTCTLRSGINVGPYRIYWYQQKPG | 23 |
| | SPPQYLLNYKSDSDKQQGSGVPSRFSGSKDASANAGVLLISGL | |
| | RSEDEADYYCMIWHSSAAVFGGGTQLTVLS | |

*The anti-mesothelin antibody is also referred to herein as P4.

In some embodiments, the anti-mesothelin antibody comprises a heavy chain variable region (V_{H}) comprising a sequence having about 80%, 85%, 90%, 95%, 96%, 97%, 98%, 99%, or 100% sequence identity to SEQ ID NO: 22; and a light chain variable region (VL) comprising a sequence having about 80%, 85%, 90%, 95%, 96%, 97%, 98%, 99%, or 100% sequence identity to SEQ ID NO: 23. In some embodiments, the anti-mesothelin antibody comprises a heavy chain variable region (V_{H}) comprising a sequence having about 80% sequence identity to SEQ ID NO: 22; and a light chain variable region (VL) comprising a sequence having about 80% sequence identity to SEQ ID NO: 23. In some embodiments, the anti-mesothelin antibody comprises a heavy chain variable region (V_{H}) comprising a sequence having about 85% sequence identity to SEQ ID NO: 22; and a light chain variable region (VL) comprising a sequence having about 85% sequence identity to SEQ ID NO: 23. In some embodiments, the anti-mesothelin antibody comprises a heavy chain variable region (V_{H}) comprising a sequence having about 90% sequence identity to SEQ ID NO: 22; and a light chain variable region (VL) comprising a sequence having about 90% sequence identity to SEQ ID NO: 23. In some embodiments, the anti-mesothelin antibody comprises a heavy chain variable region (V_{H}) comprising a sequence having about 95% sequence identity to SEQ ID NO: 22; and a light chain variable region (VL) comprising a sequence having about 95% sequence identity to SEQ ID NO: 23. In some embodiments, the anti-mesothelin antibody comprises a heavy chain variable region (V_{H}) comprising a sequence having about 96% sequence identity to SEQ ID NO: 22; and a light chain variable region (VL) comprising a sequence having about 96% sequence identity to SEQ ID NO: 23. In some embodiments, the anti-mesothelin antibody comprises a heavy chain variable region (V_{H}) comprising a sequence having about 97% sequence identity to SEQ ID NO: 22; and a light chain variable region (VL) comprising a sequence having about 97% sequence identity to SEQ ID NO: 23. In some embodiments, the anti-mesothelin antibody comprises a heavy chain variable region (V_{H}) comprising a sequence having about 98% sequence identity to SEQ ID NO: 22; and a light chain variable region (VL) comprising a sequence having about 98% sequence identity to SEQ ID NO: 23. In some embodiments, the anti-mesothelin antibody comprises a heavy chain variable region (V_{H}) comprising a sequence having about 99% sequence identity to SEQ ID NO: 22; and a light chain variable region (VL) comprising a sequence having about 99% sequence identity to SEQ ID NO: 23. The anti-mesothelin antibody can comprise a heavy chain variable region (V_{H}) comprising SEQ ID NO: 22 and a light chain variable region (VL) comprising SEQ ID NO: 23. The anti-mesothelin antibody can comprise a heavy chain variable region (V_{H}) consisting of SEQ ID NO: 22 and a light chain variable region (VL) consisting of SEQ ID NO: 23.

In some embodiments, one or more residues within the framework region are modified in the anti-mesothelin antibody, generating the 80%, 85%, 90%, 95%, 96%, 97%, 98%, or 99% sequence identity in either the VH or the VL region. The term ‘framework region” refers to the region of the antibody that excludes the complementarity-determining regions (CDRs). In some embodiments, the anti-mesothelin antibody comprises one or more modifications within the framework region and has a sequence comprising 80%, 85%, 90%, 95%, 96%, 97%, 98%, or 99% sequence identity to SEQ ID NO: 22. In some embodiments, the anti-mesothelin antibody comprises one or more modifications within the framework region and has a sequence comprising 85% sequence identity to SEQ ID NO: 22. In some embodiments, the anti-mesothelin antibody comprises one or more modifications within the framework region and has a sequence comprising 90% sequence identity to SEQ ID NO: 22. In some embodiments, the anti-mesothelin antibody comprises one or more modifications within the framework region and has a sequence comprising 95% sequence identity to SEQ ID NO: 22. In some embodiments, the anti-mesothelin antibody comprises one or more modifications within the framework region and has a sequence comprising 96% sequence identity to SEQ ID NO: 22. In some embodiments, the anti-mesothelin antibody comprises one or more modifications within the framework region and has a sequence comprising 97% sequence identity to SEQ ID NO: 22. In some embodiments, the anti-mesothelin antibody comprises one or more modifications within the framework region and has a sequence comprising 98% sequence identity to SEQ ID NO: 22. In some embodiments, the anti-mesothelin antibody comprises one or more modifications within the framework region and has a sequence comprising 99% sequence identity to SEQ ID NO: 22. In some embodiments, the anti-mesothelin antibody comprises one or more modifications within the framework region and has a sequence comprising 80%, 85%, 90%, 95%, 96%, 97%, 98%, or 99% sequence identity to SEQ ID NO: 23. In some embodiments, the anti-mesothelin antibody comprises one or more modifications within the framework region and has a sequence comprising 85% sequence identity to SEQ ID NO: 23. In some embodiments, the anti-mesothelin antibody comprises one or more modifications within the framework region and has a sequence comprising 90% sequence identity to SEQ ID NO: 23. In some embodiments, the anti-mesothelin antibody comprises one or more modifications within the framework region and has a sequence comprising 95% sequence identity to SEQ ID NO: 23. In some embodiments, the anti-mesothelin antibody comprises one or more modifications within the framework region and has a sequence comprising 96% sequence identity to SEQ ID NO: 23. In some embodiments, the anti-mesothelin antibody comprises one or more modifications within the framework region and has a sequence comprising 97% sequence identity to SEQ ID NO: 23. In some embodiments, the anti-mesothelin antibody comprises one or more modifications within the framework region and has a sequence comprising 98% sequence identity to SEQ ID NO: 23. In some embodiments, the anti-mesothelin antibody comprises one or more modifications within the framework region and has a sequence comprising 99% sequence identity to SEQ ID NO: 23.

In some embodiments, the anti-mesothelin antibody comprises a single-chain variable fragment (scFv) format. In some embodiments, the anti-mesothelin scFv antibody comprises a VH comprising or consisting of CDRH1 as set forth in SEQ ID NO: 16, CDRH2 as set forth in SEQ ID NO: 17, and CDRH3 as set forth in SEQ ID NO: 18; and a VL comprising or consisting of CDRL1 as set forth in SEQ ID NO: 19, CDRL2 as set forth in SEQ ID NO: 20, and CDRL3 as set forth in SEQ ID NO: 21. In some embodiments, the anti-mesothelin scFv antibody comprises a VH comprising a sequence having about 80%, 85%, 90%, 95%, 96%, 97%, 98%, 99%, or 100% sequence identity to SEQ ID NO: 22; and a VL comprising a sequence having about 80%, 85%, 90%, 95%, 96%, 97%, 98%, 99%, or 100% sequence identity to SEQ ID NO: 23.

In some embodiments, the VH and the VL of the anti-mesothelin scFv antibody are connected through a peptide linker. The peptide linker can include 3 or more amino acid residues, for example, from about 3 to about 30, from about 3 to about 25, from 3 to about 20, from about 4 to about 30, from about 4 to about 20, from about 4 to about 10, from about 5 to about 30, from about 5 to about 20, or from about 5 to about 10. The peptide linker can include 3, 4, 5, 6, 7, 8, 9, 10, 15, 20, 25, or 30 amino acid residues.

The peptide linker can include a plurality of poly-alanines, poly-glycines, or a mixture of alanine and glycine residues. The peptide linker can include a (Gly₄Ser) n linker, in which n is an integer from 1 to 30 (SEQ ID NO: 76), preferably 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, further preferably 15, 16, 17, 18, 19 or 20. In some instances, the peptide linker comprises GILGSGGGGSGGGGSGGGGS (SEQ ID NO: 45) the peptide linker comprises GGGGSGGGGSGGGGS (SEQ ID NO: 30). In some instances, the peptide linker comprises GSGGGGGGGGSGGGGS (SEQ ID NO: 41). In some instances, the peptide linker comprises SEQ ID NO: 45.

In some embodiments, the anti-mesothelin scFv antibody comprises a sequence comprising about 80%, 85%, 90%, 95%, 96%, 97%, 98%, or 99% sequence identity to QVQLQQSGPGLVTPSQTLSLTCAISGDSVSSNSATWNWIRQSPSRGLEWLGRTY YRSKWYNDYAVSVKSRMSLNPDTSKNQFSLQLNSVTPEDTAVYYCARGMMTY YYGMDVWGQGTTVTVSSGILGSGGGGSGGGGSGGGGSQPVLTQSSSLSASPGA SASLTCTLRSGINVGPYRIYWYQQKPGSPPQYLLNYKSDSDKQQGSGVPSRFSG SKDASANAGVLLISGLRSEDEADYYCMIWHSSAAVFGGGTQLTVLS (SEQ ID NO: 1), in which the linker is italicized. The anti-mesothelin scFv is also referred to herein as P4. In some embodiments, the anti-mesothelin scFv antibody comprises a sequence comprising about 85% sequence identity to SEQ ID NO: 1. In some embodiments, the anti-mesothelin scFv antibody comprises a sequence comprising about 90% sequence identity to SEQ ID NO: 1. In some embodiments, the anti-mesothelin scFv antibody comprises a sequence comprising about 95% sequence identity to SEQ ID NO: 1. In some embodiments, the anti-mesothelin scFv antibody comprises a sequence comprising about 96% sequence identity to SEQ ID NO: 1. In some embodiments, the anti-mesothelin scFv antibody comprises a sequence comprising about 97% sequence identity to SEQ ID NO: 1. In some embodiments, the anti-mesothelin scFv antibody comprises a sequence comprising about 98% sequence identity to SEQ ID NO: 1. In some embodiments, the anti-mesothelin scFv antibody comprises a sequence comprising about 99% sequence identity to SEQ ID NO: 1. In some embodiments, the anti-mesothelin scFv antibody comprises SEQ ID NO: 1. In some embodiments, the anti-mesothelin scFv antibody consists of SEQ ID NO: 1.

### B. Signaling Peptide for CAR

In some embodiments, a chimeric antigen receptor (CAR) disclosed herein comprises a signaling peptide (e.g., as a leader sequence). The signaling peptide can localize the CAR to the surface of the cell. The signaling peptide can include polypeptides of an immune globulin heavy chain, an immunoglobulin light chain, CD8, T cell receptor α and β chains, CD3ζ, CD28, CD3E, CD45, CD4, CD5, CD8, CD9, CD16, CD22, CD33, CD37, CD64, CD80, CD86, CD134, CD137, ICOS, CD154, or a GITR-derived signal peptide (leader sequence).

In some embodiments, the signaling peptide can include polypeptides of an immune globulin heavy chain. In some embodiments, the signaling peptide comprises a sequence comprising about 80%, 85%, 90%, 95%, 96%, 97%, 98%, 99%, or 100% sequence identity to MDWTWRILFLVAAATGAHS (SEQ ID NO: 2). In some embodiments, the signaling peptide comprises a sequence comprising about 85% sequence identity to SEQ ID NO: 2. In some embodiments, the signaling peptide comprises a sequence comprising about 90% sequence identity to SEQ ID NO: 2. In some embodiments, the signaling peptide comprises a sequence comprising about 95% sequence identity to SEQ ID NO: 2. In some embodiments, the signaling peptide comprises a sequence comprising about 96% sequence identity to SEQ ID NO: 2. In some embodiments, the signaling peptide comprises a sequence comprising about 97% sequence identity to SEQ ID NO: 2. In some embodiments, the signaling peptide comprises a sequence comprising about 98% sequence identity to SEQ ID NO: 2. In some embodiments, the signaling peptide comprises a sequence comprising about 99% sequence identity to SEQ ID NO: 2. In some embodiments, the signaling peptide comprises SEQ ID NO: 2. In some embodiments, the signaling peptide consists of SEQ ID NO: 2.

### C. Transmembrane Regions

In some embodiments, the anti-mesothelin antibody is linked to one or more transmembrane and intracellular signaling domains. The transmembrane region can be derived from either a natural or synthetic source. Exemplary transmembrane regions can include polypeptides of transmembrane regions derived from CD8, T cell receptor a and B chains, CD3ζ, CD28, CD3E (CD3 epsilon), CD45, CD4, CD5, CD8, CD9, CD16, CD22, CD33, CD37, CD64, CD80, CD86, CD134, CD137, ICOS, CD154, and GITR. In some embodiments, the transmembrane region comprises a CD8 transmembrane region (e.g., human CD8 transmembrane region).

In some embodiments, transmembrane region is derived from CD8. In some embodiments, the transmembrane region comprises a sequence comprising about 80%, 85%, 90%, 95%, 96%, 97%, 98%, 99%, or 100% sequence identity to IYIWAPLAGTCGVLLLSLVITLYCN (SEQ ID NO: 27). In some embodiments, the transmembrane region comprises a sequence comprising about 85% sequence identity to SEQ ID NO: 27. In some embodiments, the transmembrane region comprises a sequence comprising about 90% sequence identity to SEQ ID NO: 27. In some embodiments, the transmembrane region comprises a sequence comprising about 95% sequence identity to SEQ ID NO: 27. In some embodiments, the transmembrane region comprises a sequence comprising about 96% sequence identity to SEQ ID NO: 27. In some embodiments, the transmembrane region comprises a sequence comprising about 97% sequence identity to SEQ ID NO: 27. In some embodiments, the transmembrane region comprises a sequence comprising about 98% sequence identity to SEQ ID NO: 27. In some embodiments, the transmembrane region comprises a sequence comprising about 99% sequence identity to SEQ ID NO: 27. In some embodiments, the transmembrane region comprises SEQ ID NO: 27. In some embodiments, the transmembrane region consists of SEQ ID NO: 27. In some embodiments, the transmembrane region comprises a sequence comprising about 80%, 85%, 90%, 95%, 96%, 97%, 98%, 99%, or 100% sequence identity to IYIWAPLAGTCGVLLLSLVITLYC (SEQ ID NO: 63). In some embodiments, the transmembrane region comprises a sequence comprising about 85% sequence identity to SEQ ID NO: 63. In some embodiments, the transmembrane region comprises a sequence comprising about 90% sequence identity to SEQ ID NO: 63. In some embodiments, the transmembrane region comprises a sequence comprising about 95% sequence identity to SEQ ID NO: 63. In some embodiments, the transmembrane region comprises a sequence comprising about 96% sequence identity to SEQ ID NO: 63. In some embodiments, the transmembrane region comprises a sequence comprising about 97% sequence identity to SEQ ID NO: 63. In some embodiments, the transmembrane region comprises a sequence comprising about 98% sequence identity to SEQ ID NO: 63. In some embodiments, the transmembrane region comprises a sequence comprising about 99% sequence identity to SEQ ID NO: 63. In some embodiments, the transmembrane region comprises SEQ ID NO: 63. In some embodiments, the transmembrane region consists of SEQ ID NO: 63.

### D. Extracellular Hinge Region

An extracellular hinge region comprising or consisting of an arbitrary oligopeptide or polypeptide may be located between the cell surface molecule recognizing mesothelin and the transmembrane region. Examples of the length of the extracellular hinge region can include 1 to 100 amino acid residues, preferably 10 to 70, 10 to 50, or 10 to 30 amino acid residues. Exemplary extracellular hinge regions can include hinge regions derived from CD8, CD28, and CD4, and an immune globulin hinge region. In some embodiments, the hinge region comprises the hinge region of human CD8.

In some embodiments, the extracellular hinge region is a CD8 hinge region comprising a sequence having about 80%, 85%, 90%, 95%, 96%, 97%, 98%, 99%, or 100% sequence identity to PTTTPAPRPPTPAPTIASQPLSLRPEACRPAAGGAVHTRGLDFACD (SEQ ID NO: 26). In some embodiments, the CD8 hinge region comprises a sequence having about 85% sequence identity to SEQ ID NO: 26. In some embodiments, the CD8 hinge region comprises a sequence having about 90% sequence identity to SEQ ID NO: 26. In some embodiments, the CD8 hinge region comprises a sequence having about 95% sequence identity to SEQ ID NO: 26. In some embodiments, the CD8 hinge region comprises a sequence having about 96% sequence identity to SEQ ID NO: 26. In some embodiments, the CD8 hinge region comprises a sequence having about 97% sequence identity to SEQ ID NO: 26. In some embodiments, the CD8 hinge region comprises a sequence having about 98% sequence identity to SEQ ID NO: 26. In some embodiments, the CD8 hinge region comprises a sequence having about 99% sequence identity to SEQ ID NO: 26. In some embodiments, the CD8 hinge region comprises SEQ ID NO: 26. In some embodiments, the CD8 hinge region consists of SEQ ID NO: 26. In some embodiments, the CD8 hinge region comprises a sequence having about 80%, 85%, 90%, 95%, 96%, 97%, 98%, 99%, or 100% sequence identity to TTTPAPRPPTPAPTIASQPLSLRPEACRPAAGGAVHTRGLDFACD (SEQ ID NO: 64). In some embodiments, the CD8 hinge region comprises a sequence having about 85% sequence identity to SEQ ID NO: 64. In some embodiments, the CD8 hinge region comprises a sequence having about 90% sequence identity to SEQ ID NO: 64. In some embodiments, the CD8 hinge region comprises a sequence having about 95% sequence identity to SEQ ID NO: 64. In some embodiments, the CD8 hinge region comprises a sequence having about 96% sequence identity to SEQ ID NO: 64. In some embodiments, the CD8 hinge region comprises a sequence having about 97% sequence identity to SEQ ID NO: 64. In some embodiments, the CD8 hinge region comprises a sequence having about 98% sequence identity to SEQ ID NO: 64. In some embodiments, the CD8 hinge region comprises a sequence having about 99% sequence identity to SEQ ID NO: 64. In some embodiments, the CD8 hinge region comprises SEQ ID NO: 64. In some embodiments, the CD8 hinge region consists of SEQ ID NO: 64.

In some embodiments, the anti-mesothelin scFv antibody is connected to the hinge region through a peptide linker. The peptide linker can include 3 or more amino acid residues, for example, from about 3 to about 30, from about 3 to about 20, from 3 to about 10, from about 4 to about 30, from about 4 to about 20, from about 4 to about 10, from about 5 to about 30, from about 5 to about 20, or from about 5 to about 10. The peptide linker can include 3, 4, 5, 6, 7, 8, 9, 10, 15, 20, 25, or 30 amino acid residues.

The peptide linker can include a plurality of poly-alanines, poly-glycines, a mixture of alanine and glycine residues, or a mixture of either alanines or glycines with one or more additional amino acids. In some embodiments, the peptide linker comprises ArgAlaAlaAla (“RAAA”) (SEQ ID NO: 4). In some embodiments, the peptide linker is ArgAlaAlaAla (“RAAA”) (SEQ ID NO: 4). In some embodiments, the peptide linker comprises AlaAlaAla (“AAA”). In some embodiments, the peptide linker is a triple alanine linker or AlaAlaAla (“AAA”).

In some embodiments, the anti-mesothelin scFv antibody is connected to the hinge region without a linker.

### E. Intracellular Signaling Regions (Costimulatory Domain)

In some embodiments, the CAR comprises one or more intracellular signaling regions. The intracellular signaling regions (Note the intracellular signaling regions are also referred to herein as “costimulatory domain”) can comprise a region capable of transducing signals into the cell when the cell surface molecule recognizes mesothelin. The intracellular signaling region can comprise at least one or more members selected from intracellular regions of polypeptides of CD28, 4-1BB (CD137), GITR, CD27, OX40, HVEM, CD3ζ, or Fc receptor-associated γ chain. In some embodiments, the intracellular signaling region comprises a polypeptide of a CD28 intracellular region (costimulatory domain), a polypeptide of a 4-1BB intracellular region (costimulatory domain), a polypeptide of a CD3 intracellular region, or a combination thereof.

In some embodiments, the CAR comprises a 4-1BB intracellular region (costimulatory domain). In some embodiments, the 4-1BB intracellular region (costimulatory domain) comprises a sequence comprising about 80%, 85%, 90%, 95%, 96%, 97%, 98%, 99%, or 100% sequence identity to KRGRKKLLYIFKQPFMRPVQTTQEEDGCSCRFPEEEEGGCEL (SEQ ID NO: 24). In some embodiments, the 4-1BB intracellular region comprises a sequence comprising about 85% sequence identity to SEQ ID NO: 24. In some embodiments, the 4-1BB intracellular region comprises a sequence comprising about 90% sequence identity to SEQ ID NO: 24. In some embodiments, the 4-1BB intracellular region comprises a sequence comprising about 95% sequence identity to SEQ ID NO: 24. In some embodiments, the 4-1BB intracellular region comprises a sequence comprising about 96% sequence identity to SEQ ID NO: 24. In some embodiments, the 4-1BB intracellular region comprises a sequence comprising about 97% sequence identity to SEQ ID NO: 24. In some embodiments, the 4-1BB intracellular region comprises a sequence comprising about 98% sequence identity to SEQ ID NO: 24. In some embodiments, the 4-1BB intracellular region comprises a sequence comprising about 99% sequence identity to SEQ ID NO: 24. In some embodiments, the 4-1BB intracellular region comprises SEQ ID NO: 24. In some embodiments, the 4-1BB intracellular region consists of SEQ ID NO: 24.

In some embodiments, the CAR further comprises a CD3 intracellular region. In some embodiments, the CD3ζintracellular region comprises a sequence comprising about 80%, 85%, 90%, 95%, 96%, 97%, 98%, 99%, or 100% sequence identity to RVKFSRSADAPAYQQGQNQLYNELNLGRREEYDVLDKRRGRDPEMGGKPRRK NPQEGLYNELQKDKMAEAYSEIGMKGERRRGKGHDGLYQGLSTATKDTYDAL HMQALPPR (SEQ ID NO: 25). In some embodiments, the CD33 intracellular region comprises a sequence comprising about 85% sequence identity to SEQ ID NO: 25. In some embodiments, the CD3ζintracellular region comprises a sequence comprising about 90% sequence identity to SEQ ID NO: 25. In some embodiments, the CD34 intracellular region comprises a sequence comprising about 95% sequence identity to SEQ ID NO: 25. In some embodiments, the CD3 intracellular region comprises a sequence comprising about 96% sequence identity to SEQ ID NO: 25. In some embodiments, the CD3 intracellular region comprises a sequence comprising about 97% sequence identity to SEQ ID NO: 25. In some embodiments, the CD3ζintracellular region comprises a sequence comprising about 98% sequence identity to SEQ ID NO: 25. In some embodiments, the CD3 intracellular region comprises a sequence comprising about 99% sequence identity to SEQ ID NO: 25. In some embodiments, the CD3ζintracellular region comprises SEQ ID NO: 25. In some embodiments, the CD3ζintracellular region consists of SEQ ID NO: 25.

### IL-15

Interleukin 15 (IL-15) is involved in lymphocyte development, differentiation, and homeostasis. IL-15 also stimulates CD8 T cells and induces natural killer (NK) cell activation. Within the tumor microenvironment, expression of IL-15 has been correlated with enhanced antitumor responses. Furthermore, expression of IL-15 has shown benefits during ex vivo expansion of T cells and enhances the potency of CAR-T cells.

As used herein, the term “IL-15” encompasses not only full-length IL-15 protein but also fragments so long as the function of IL-15 in the effect of the present invention is retained. Further, the term “IL-15” encompasses wild-type IL-15 and variants thereof, comprising one or more mutations. In some cases, the mutations comprise deletions, substitutions, and/or additions. For example, the IL-15 may comprise one or more substitutions. The IL-15 may also comprise a truncation of 1, 2, 3, 4, 5, 6, 7, 8, 9, 10, 15, 20, or more amino acid residues, in which the truncation may be located at the N-terminus, the C-terminus, or an internal region of IL-15. Furthermore, IL-15 or a variant thereof may be linked to another protein to form a fusion protein (e.g., linked to the whole or a portion of IL-15Ra). IL-15 may be a membrane bounded protein or secreting protein.

A. mIL15/Ra

In some instances, the IL-15 comprises a membrane bound IL-15 fused to the IL-15 receptor α chain (IL-15Rα). The fusion IL-15 protein is referred to herein as mIL15/Ra. The IL-15Rα encompasses the entire wild-type IL-15Rα (without the signal peptide), but also encompasses a functional fragment thereof (e.g., an extracellular domain or a sushi domain of IL-15Rα), and variants of IL-15Rα, optionally comprising one or more substitutions, deletions, or additions.

In some instances, the mIL15/Ra fusion protein comprises an IL-15Rα polypeptide fused to the C-terminus of a membrane bound IL-15 polypeptide, either directly or indirectly via a linker. The peptide linker can include 1 or more amino acid residues, for example, from about 1 to about 30, from about 3 to about 20, from 3 to about 10, from about 4 to about 30, from about 4 to about 20, from about 4 to about 10, from about 5 to about 30, from about 5 to about 20, or from about 5 to about 10. The peptide linker can include 3, 4, 5, 6, 7, 8, 9, 10, 15, 20, 25, or 30 amino acid residues.

The peptide linker can include a plurality of poly-alanines, poly-glycines, or a mixture of alanine and glycine residues. The peptide linker can include a (Gly₄Ser) n linker, in which n is an integer from 1 to 30 (SEQ ID NO: 76), preferably 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, further preferably 15, 16, 17, 18, 19, 20, 21, 22, 23, 24, 25, 26, 27, 28, 29 or 30. In some instances, the peptide linker comprises SGGGSGGGGSGGGGGGGGSGGGSLQ (SEQ ID NO: 32). In some instances, the peptide linker comprises SGGSGGGGSGGGSGGGGSLQ (SEQ ID NO: 33). In some instances, the peptide linker comprises GGGGSGGGGSGGGGS (SEQ ID NO: 30). In some instances, the peptide linker comprises GSGGGGSGGGGSGGGGS (SEQ ID NO: 41). In some instances, the peptide linker comprises SEQ ID NO: 45.

In some embodiments, an immune cell described herein expresses mIL15/Ra. In some embodiments, the mIL15/Ra comprises a sequence comprising about 80%, 85%, 90%, 95%, 96%, 97%, 98%, 99%, or 100% sequence identity to NWVNVISDLKKIEDLIQSMHIDATLYTESDVHPSCKVTAMKCFLLELQVISLESG DASIHDTVENLIILANNSLSSNGNVTESGCKECEELEEKNIKEFLQSFVHIVQMFI NTSSGGGSGGGGSGGGGSGGGGSGGGSLQITCPPPMSVEHADIWVKSYSLYSRE RYICNSGFKRKAGTSSLTECVLNKATNVAHWTTPSLKCIRDPALVHQRPAPPST VTTAGVTPQPESLSPSGKEPAASSPSSNNTAATTAAIVPGSQLMPSKSPSTGTTEI SSHESSHGTPSQTTAKNWELTASASHQPPGVYPQGHSDTTVAISTSTVLLCGLS AVSLLACYLKSRQTPPLASVEMEAMEALPVTWGTSSRDEDLENCSHHL (SEQ ID NO: 8). In some embodiments, the mIL15/Ra comprises a sequence comprising about 85% sequence identity to SEQ ID NO: 8. In some embodiments, the mIL15/Ra comprises a sequence comprising about 90% sequence identity to SEQ ID NO: 8. In some embodiments, the mIL15/Ra comprises a sequence comprising about 95% sequence identity to SEQ ID NO: 8. In some embodiments, the mIL15/Ra comprises a sequence comprising about 96% sequence identity to SEQ ID NO: 8. In some embodiments, the mIL15/Ra comprises a sequence comprising about 97% sequence identity to SEQ ID NO: 8. In some embodiments, the mIL15/Ra comprises a sequence comprising about 98% sequence identity to SEQ ID NO: 8. In some embodiments, the mIL15/Ra comprises a sequence comprising about 99% sequence identity to SEQ ID NO: 8. In some embodiments, the mIL15/Ra comprises SEQ ID NO: 8. In some embodiments, the mIL15/Ra consists of SEQ ID NO: 8.

In some instances, the mIL15/Ra further comprises an IL-2 signal peptide (IL2sp). In some cases, the IL2sp comprises MYRMQLLSCIALSLALVTNS (SEQ ID NO: 6). In some cases, the IL2sp consists of SEQ ID NO: 6.

In some cases, an immune cell described herein expresses an IL2sp-mIL15/Ra polypeptide comprising about 80%, 85%, 90%, 95%, 96%, 97%, 98%, 99%, or 100% sequence identity to MYRMQLLSCIALSLALVTNSATSNWVNVISDLKKIEDLIQSMHIDATLYTESDV HPSCKVTAMKCFLLELQVISLESGDASIHDTVENLIILANNSLSSNGNVTESGCK ECEELEEKNIKEFLQSFVHIVQMFINTSSGGGSGGGGSGGGGSGGGGSGGGSLQI TCPPPMSVEHADIWVKSYSLYSRERYICNSGFKRKAGTSSLTECVLNKATNVAH WTTPSLKCIRDPALVHQRPAPPSTVTTAGVTPQPESLSPSGKEPAASSPSSNNTA ATTAAIVPGSQLMPSKSPSTGTTEISSHESSHGTPSQTTAKNWELTASASHQPPG VYPQGHSDTTVAISTSTVLLCGLSAVSLLACYLKSRQTPPLASVEMEAMEALPV TWGTSSRDEDLENCSHHL (SEQ ID NO: 28). In some cases, the IL2sp-mIL15/Ra polypeptide comprises about 85% sequence identity to SEQ ID NO: 28. In some cases, the IL2sp-mIL15/Ra polypeptide comprises about 90% sequence identity to SEQ ID NO: 28. In some cases, the IL2sp-mIL15/Ra polypeptide comprises about 95% sequence identity to SEQ ID NO: 28. In some cases, the IL2sp-mIL15/Ra polypeptide comprises about 96% sequence identity to SEQ ID NO: 28. In some cases, the IL2sp-mIL15/Ra polypeptide comprises about 97% sequence identity to SEQ ID NO: 28. In some cases, the IL2sp-mIL15/Ra polypeptide comprises about 98% sequence identity to SEQ ID NO: 28. In some cases, the IL2sp-mIL15/Ra polypeptide comprises about 99% sequence identity to SEQ ID NO: 28. In some cases, the IL2sp-mIL15/Ra polypeptide comprises SEQ ID NO: 28. In some cases, the IL2sp-mIL15/Ra polypeptide consists of SEQ ID NO: 28.

### B. Sushi15

In some instances, the IL-15 comprises an IL-15 receptor α chain (IL-15Rα) sushi domain fused to an IL-15. This fusion IL-15 protein is referred to herein as sushi15. The IL-15Rα sushi domain is fused to the C-terminus of the IL-15 polypeptide, either directly or indirectly via a linker.

In some case, the sushi domain of the IL-15Rα is fused to the IL-15 polypeptide indirectly via a linker. The peptide linker can include 1 or more amino acid residues, for example, from about 1 to about 30, from about 3 to about 20, from 3 to about 10, from about 4 to about 30, from about 4 to about 20, from about 4 to about 10, from about 5 to about 30, from about 5 to about 20, or from about 5 to about 10. The peptide linker can include 3, 4, 5, 6, 7, 8, 9, 10, 15, 20, 25, or 30 amino acid residues.

The peptide linker can include a plurality of poly-alanines, poly-glycines, or a mixture of alanine and glycine residues. The peptide linker can include a (Gly₄Ser) n linker, in which n is an integer from 1 to 30 (SEQ ID NO: 76), preferably 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, further preferably 15, 16, 17, 18, 19, 20, 21, 22, 23, 24, 25, 26, 27, 28, 29 or 30. In some instances, the peptide linker comprises SEQ ID NO: 32. In some instances, the peptide linker comprises SEQ ID NO: 33. In some instances, the peptide linker comprises SEQ ID NO: 30. In some instances, the peptide linker comprises SEQ ID NO: 41. In some instances, the peptide linker comprises SEQ ID NO: 45.

In some embodiments, an immune cell described herein expresses sushi15. In some embodiments, the sushi15 comprises a sequence comprising about 80%, 85%, 90%, 95%, 96%, 97%, 98%, 99%, or 100% sequence identity to ITCPPPMSVEHADIWVKSYSLYSRERYICNSGFKRKAGTSSLTECVLNKATNVA HWTTPSLKCIRSGGGSGGGGSGGGGSGGGGSGGGSLQNWVNVISDLKKIEDLIQ SMHIDATLYTESDVHPSCKVTAMKCFLLELQVISLESGDASIHDTVENLIILANN SLSSNGNVTESGCKECEELEEKNIKEFLQSFVHIVQMFINTS (SEQ ID NO: 10). In some cases, the sushi15 comprises a sequence comprising about 85% sequence identity to SEQ ID NO: 10. In some cases, the sushi15 comprises a sequence comprising about 90% sequence identity to SEQ ID NO: 10. In some cases, the sushi15 comprises a sequence comprising about 95% sequence identity to SEQ ID NO: 10. In some cases, the sushi15 comprises a sequence comprising about 96% sequence identity to SEQ ID NO: 10. In some cases, the sushi15 comprises a sequence comprising about 97% sequence identity to SEQ ID NO: 10. In some cases, the sushi15 comprises a sequence comprising about 98% sequence identity to SEQ ID NO: 10. In some cases, the sushi15 comprises a sequence comprising about 99% sequence identity to SEQ ID NO: 10. In some cases, the sushi15 comprises SEQ ID NO: 10. In some cases, the sushi15 consists of SEQ ID NO: 10.

In some instances, the sushi15 further comprises an IL-15Rα signal peptide (IL15Rasp). In some cases, the IL15Rasp comprises MAPRRARGCRTLGLPALLLLLLLRPPATRG (SEQ ID NO: 7). In some cases, the IL15Rasp consists of SEQ ID NO: 7.

In some cases, an immune cell described herein expresses an IL15Rasp-sushi 15 polypeptide comprising about 80%, 85%, 90%, 95%, 96%, 97%, 98%, 99%, or 100% sequence identity to MAPRRARGCRTLGLPALLLLLLLRPPATRGITCPPPMSVEHADIWVKSYSLYSR ERYICNSGFKRKAGTSSLTECVLNKATNVAHWTTPSLKCIRSGGGSGGGGSGGG GSGGGGSGGGSLONWVNVISDLKKIEDLIQSMHIDATLYTESDVHPSCKVTAMK CFLLELQVISLESGDASIHDTVENLIILANNSLSSNGNVTESGCKECEELEEKNIK EFLQSFVHIVQMFINTS (SEQ ID NO: 29). In some cases, the IL15Rasp-sushi15 polypeptide comprises about 85% sequence identity to SEQ ID NO: 29. In some cases, the IL15Rasp-sushi15 polypeptide comprises about 90% sequence identity to SEQ ID NO: 29. In some cases, the IL15Rasp-sushi15 polypeptide comprises about 95% sequence identity to SEQ ID NO: 29. In some cases, the IL15Rasp-sushi15 polypeptide comprises about 96% sequence identity to SEQ ID NO: 29. In some cases, the IL15Rasp-sushi15 polypeptide comprises about 97% sequence identity to SEQ ID NO: 29. In some cases, the IL15Rasp-sushi15 polypeptide comprises about 98% sequence identity to SEQ ID NO: 29. In some cases, the IL15Rasp-sushi15 polypeptide comprises about 99% sequence identity to SEQ ID NO: 29. In some cases, the IL15Rasp-sushi15 polypeptide comprises SEQ ID NO: 29. In some cases, the IL15Rasp-sushi15 polypeptide consists of SEQ ID NO: 29.

C. mIL15/Ra-LSP

In some instances, the IL-15 comprises a membrane bound IL-15 fused to an IL-15 receptor α chain (IL-15Rα), IL15 propeptide and long signal peptide of IL-15 (LSP) fusion protein. This fusion IL-15 protein is referred to herein as mIL15/Ra-LSP. The IL-15Rα polypeptide comprises a full-length IL-15Rα chain without the signal peptide. The IL-15Rα is fused to the C-terminus of the IL-15 polypeptide, either directly or indirectly via a linker. The IL15 propeptide is fused to the C-terminus of LSP, either directly or indirectly. IL15 propeptide is fused to the C-terminus of IL-15Rα polypeptide, either directly or indirectly. In some cases, the IL15 propeptide comprises GIHVFILGCFSAGLPKTEA (SEQ ID NO: 48). In some cases, the IL15 propetide consists of SEQ ID NO: 48. In some cases LSP comprises MRISKPHLRSISIQCYLCLLLNSHFLTEA (SEQ ID NO: 47). In some cases, the LSP consists of SEQ ID NO: 47.

In some case, the IL-15Rα is fused to the C-terminus of the IL-15 polypeptide indirectly via a linker. The peptide linker can include 1 or more amino acid residues, for example, from about 1 to about 30, from about 3 to about 20, from 3 to about 10, from about 4 to about 30, from about 4 to about 20, from about 4 to about 10, from about 5 to about 30, from about 5 to about 20, or from about 5 to about 10. The peptide linker can include 3, 4, 5, 6, 7, 8, 9, 10, 15, 20, 25, or 30 amino acid residues.

The peptide linker can include a plurality of poly-serines, poly-glycines, or a mixture of serine and glycine residues. The peptide linker can include a (Gly₄Ser) n linker, in which n is an integer from 1 to 30 (SEQ ID NO: 76), preferably 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, further preferably 15, 16, 17, 18, 19, 20, 21, 22, 23, 24, 25, 26, 27, 28, 29 or 30. In some instances, the peptide linker comprises SEQ ID NO: 32. In some instances, the peptide linker comprises SEQ ID NO: 33. In some instances, the peptide linker comprises SEQ ID NO: 30. In some instances, the peptide linker comprises SEQ ID NO: 41. In some instances, the peptide linker comprises SEQ ID NO: 45.

In some embodiments, an immune cell described herein expresses mIL15/Ra-LSP. In some embodiments, the mIL15/Ra-LSP comprises a sequence comprising about 80%, 85%, 90%, 95%, 96%, 97%, 98%, 99%, or 100% sequence identity to MRISKPHLRSISIQCYLCLLLNSHFLTEAGIHVFILGCFSAGLPKTEANWVNVISD LKKIEDLIQSMHIDATLYTESDVHPSCKVTAMKCFLLELQVISLESGDASIHDTV ENLIILANNSLSSNGNVTESGCKECEELEEKNIKEFLQSFVHIVQMFINTSSGGGS GGGGSGGGGSGGGSLQITCPPPMSVEHADIWVKSYSLYSRERYICNSGF KRKAGTSSLTECVLNKATNVAHWTTPSLKCIRDPALVHQRPAPPSTVTTAGVTP QPESLSPSGKEPAASSPSSNNTAATTAAIVPGSQLMPSKSPSTGTTEISSHESSHG TPSQTTAKNWELTASASHQPPGVYPQGHSDTTVAISTSTVLLCGLSAVSLLACY LKSRQTPPLASVEMEAMEALPVTWGTSSRDEDLENCSHHL (SEQ ID NO: 9). In some cases, the mIL15/Ra-LSP comprises a sequence comprising about 85% sequence identity to SEQ ID NO: 9. In some cases, the mIL15/Ra-LSP comprises a sequence comprising about 90% sequence identity to SEQ ID NO: 9. In some cases, the mIL15/Ra-LSP comprises a sequence comprising about 95% sequence identity to SEQ ID NO: 9. In some cases, the mIL15/Ra-LSP comprises a sequence comprising about 96% sequence identity to SEQ ID NO: 9. In some cases, the mIL15/Ra-LSP comprises a sequence comprising about 97% sequence identity to SEQ ID NO: 9. In some cases, the mIL15/Ra-LSP comprises a sequence comprising about 98% sequence identity to SEQ ID NO: 9. In some cases, the mIL15/Ra-LSP comprises a sequence comprising about 99% sequence identity to SEQ ID NO: 9. In some cases, the mIL15/Ra-LSP comprises SEQ ID NO: 9. In some cases, the mIL15/Ra-LSP consists of SEQ ID NO: 9.

In some instances, the mIL15/Ra-LSP further comprises an IL-2 signal peptide (IL2sp). In some cases, the IL2sp comprises SEQ ID NO: 6. In some cases, the IL2sp consists of SEQ ID NO: 6.

D. sIL15-LSP

In some embodiments, the IL-15 comprises an IL-15 polypeptide, IL-15 propeptide and long signal peptide of IL-15 (LSP) fusion protein. This fusion IL-15 protein is referred to herein as sIL15. The IL15 propeptide is fused to the C-terminus of LSP, either directly or indirectly. IL-15 polypeptide is fused to the C-terminus of IL-15 propeptide, either directly or indirectly. In some cases, the IL15 propeptide comprises SEQ ID NO: 48. In some cases, the IL15 propetide consists of SEQ ID NO: 48. In some cases LSP comprises SEQ ID NO: 47. In some cases, the LSP consists of SEQ ID NO: 47. In some instances, an immune cell described herein expresses sIL15. In some embodiments, the sIL15 comprises a sequence comprising about 80%, 85%, 90%, 95%, 96%, 97%, 98%, 99%, or 100% sequence identity to MRISKPHLRSISIQCYLCLLLNSHFLTEAGIHVFILGCFSAGLPKTEANWVNVISD LKKIEDLIQSMHIDATLYTESDVHPSCKVTAMKCFLLELQVISLESGDASIHDTV ENLIILANNSLSSNGNVTESGCKECEELEEKNIKEFLQSFVHIVQMFINTS (SEQ ID NO: 11). In some cases, the sIL15 comprises a sequence comprising about 85% sequence identity to SEQ ID NO: 11. In some cases, the sIL15 comprises a sequence comprising about 90% sequence identity to SEQ ID NO: 11. In some cases, the sIL15 comprises a sequence comprising about 95% sequence identity to SEQ ID NO: 11. In some cases, the sIL15 comprises a sequence comprising about 96% sequence identity to SEQ ID NO: 11. In some cases, the sIL15 comprises a sequence comprising about 97% sequence identity to SEQ ID NO: 11. In some cases, the sIL15 comprises a sequence comprising about 98% sequence identity to SEQ ID NO: 11. In some cases, the sIL15 comprises a sequence comprising about 99% sequence identity to SEQ ID NO: 11. In some cases, the sIL15 comprises SEQ ID NO: 11. In some cases, the sIL15 consists of SEQ ID NO: 11.

### CCL19

Chemokine (C-C motif) ligand 19 (CCL19), also known as EB11 ligand chemokine (ELC) and macrophage inflammatory protein-3-beta (MIP-3-beta), plays a role in lymphocyte recirculation and homing. CCL19 is expressed by dendritic cells or macrophages of lymph nodes and has a function of initiating the migration of T cells, B cells, or mature dendritic cells via its receptor CCR7.

In certain embodiments, an immune cell described herein further expresses CCL19. In some embodiments, CCL19 comprises a sequence comprising about 80%, 85%, 90%, 95%, 96%, 97%, 98%, 99%, or 100% sequence identity to GTNDAEDCCLSVTQKPIPGYIVRNFHYLLIKDGCRVPAVVFTTLRGRQLCAPPD QPWVERIIQRLQRTSAKMKRRSS (SEQ ID NO: 13). In some embodiments, CCL19 comprises a sequence comprising about 85% sequence identity to SEQ ID NO: 13. In some embodiments, CCL19 comprises a sequence comprising about 90% sequence identity to SEQ ID NO: 13. In some embodiments, CCL19 comprises a sequence comprising about 95% sequence identity to SEQ ID NO: 13. In some embodiments, CCL19 comprises a sequence comprising about 96% sequence identity to SEQ ID NO: 13. In some embodiments, CCL19 comprises a sequence comprising about 97% sequence identity to SEQ ID NO: 13. In some embodiments, CCL19 comprises a sequence comprising about 98% sequence identity to SEQ ID NO: 13. In some embodiments, CCL19 comprises a sequence comprising about 99% sequence identity to SEQ ID NO: 13. In some embodiments, CCL19 comprises SEQ ID NO: 13. In some embodiments, CCL19 consists of SEQ ID NO: 13.

In some embodiments, the CCL19 further comprises a signal peptide, MALLLALSLLVLWTSPAPTLS (SEQ ID NO: 12) (also referred to herein as endosp). In some cases, an immune cell described herein expresses both the signal peptide SEQ ID NO: 12 and CCL19 comprising a sequence comprising about 80%, 85%, 90%, 95%, 96%, 97%, 98%, 99%, or 100% sequence identity to SEQ ID NO: 13. In some cases, CCL19 is fused to the C-terminus of endosp.

### Additional Immune Function Control Factor

The immune cell of the present invention may further express an additional immune function control factor such as CCL21, IL-2, IL-4, IL-7, IL-12, IL-13, IL-17, IL-18, IP-10, interferon-γ, MIP-1alpha, GM-CSF, M-CSF, TGF-beta, XCL1, FLT3L or TNF-alpha. In some embodiments, the additional immune function control factor comprises IL-2. In some embodiments, the additional immune function control factor comprises IL-7. In some embodiments, the additional immune function control factor comprises interferon-γ. In some embodiments, the additional immune function control factor comprises GM-CSF. In some embodiments, the additional immune function control factor comprises TGF-beta. In some embodiments, the additional immune function control factor comprises TNF-alpha. In some embodiments, the additional immune function control factor is preferably an immune function control factor other than IL-12.

### Arrangement of Each Region

In certain embodiments, disclosed herein is an isolated nucleic acid molecule comprising one or more polynucleotides that encode the engineered cell surface molecule that specifically bind to mesothelin (e.g., a CAR that specifically binds to mesothelin), IL-15, and optionally CCL19. In some embodiments, the isolated nucleic acid molecule comprises a first polynucleotide encoding a CAR comprising an antibody that specifically recognizes human mesothelin, a CD8 hinge region, a CD8 transmembrane region, a 4-1BB intracellular region, and a CD3 intracellular region; and a second polynucleotide that encodes IL-15. In some instances, the isolated nucleic acid molecule comprises a first polynucleotide encoding a CAR comprising an antibody that specifically recognizes human mesothelin, a CD8 hinge region, a CD8 transmembrane region, a 4-1BB intracellular region, and a CD3ζ intracellular region; a second polynucleotide that encodes IL-15; and a third polynucleotide that encodes CCL19. In some embodiments, the polynucleotides that encode the CAR, the IL-15, and the CCL19 are located on two or more different polynucleotides in the nucleic acid molecule. In other embodiments, the isolated nucleic acid molecule comprises the polynucleotides that encode the CAR and IL-15, the polynucleotides that encode the CAR and CCL19, or the polynucleotide that encode the CAR, IL-15, or CCL19.

In some embodiments, the polynucleotide encoding the CAR comprises a signaling peptide upstream of the antibody that specifically recognizes human mesothelin. In some embodiments, the antibody is linked to the CD8 hinge region by a peptide linker (e.g., SEQ ID NO: 41). In some embodiments, the 4-1BB intracellular region is located upstream of the CD3ζintracellular region in the polynucleotide.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS (SEQ ID NO: 3) (also referred to herein as ssV_{H}-P4). The CAR can comprise a sequence having about 85% sequence identity to SEQ ID NO: 3. The CAR can comprise a sequence having about 90% sequence identity to SEQ ID NO: 3. The CAR can comprise a sequence having about 95% sequence identity to SEQ ID NO: 3. The CAR can comprise a sequence having about 96% sequence identity to SEQ ID NO: 3. The CAR can comprise a sequence having about 97% sequence identity to SEQ ID NO: 3. The CAR can comprise a sequence having about 98% sequence identity to SEQ ID NO: 3. The CAR can comprise a sequence having about 99% sequence identity to SEQ ID NO: 3. The CAR can comprise SEQ ID NO: 3. The CAR can consist of SEQ ID NO: 3.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] TTTPAPRPPTPAPTIASQPLSLRPEACRPAAGGAVHT RGLDFACDIYIWAPLAGTCGVLLLSLVITLYC [symbol] KRGRKKLLYIFKQPFMRPVQT TQEEDGCSCRFPEEEEGGCELRVKFSRSADAPAYQQGQNQLYNELNLGRREEY DVLDKRRGRDPEMGGKPRRKNPQEGLYNELQKDKMAEAYSEIGMKGERRRG KGHDGLYQGLSTATKDTYDALHMQALPPR (SEQ ID NO: 46) (also referred to herein as ssV_{H}-P4-CAR). The CAR can comprise a sequence having about 85% sequence identity to SEQ ID NO: 46. The CAR can comprise a sequence having about 90% sequence identity to SEQ ID NO: 46. The CAR can comprise a sequence having about 95% sequence identity to SEQ ID NO: 46. The CAR can comprise a sequence having about 96% sequence identity to SEQ ID NO: 46. The CAR can comprise a sequence having about 97% sequence identity to SEQ ID NO: 46. The CAR can comprise a sequence having about 98% sequence identity to SEQ ID NO: 46. The CAR can comprise a sequence having about 99% sequence identity to SEQ ID NO: 46. The CAR can comprise SEQ ID NO: 46. The CAR can consist of SEQ ID NO: 46.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] PTTTPAPRPPTPAPTIASQPLSLRPEACRPAAGGAVHT RGLDFACDIYIWAPLAGTCGVLLLSLVITLYCNKRGRKKLLYIFKQPFMRPVQT TQEEDGCSCRFPEEEEGGCELRVKFSRSADAPAYQQGQNQLYNELNLGRREEY DVLDKRRGRDPEMGGKPRRKNPQEGLYNELQKDKMAEAYSEIGMKGERRRG KGHDGLYQGLSTATKDTYDALHMQALPPR (SEQ ID NO: 43) (also referred to herein as ssVHsp-P4-CD8hinge-TM-41BB-CD3z). The CAR can comprise a sequence having about 85% sequence identity to SEQ ID NO: 43. The CAR can comprise a sequence having about 90% sequence identity to SEQ ID NO: 43. The CAR can comprise a sequence having about 95% sequence identity to SEQ ID NO: 43. The CAR can comprise a sequence having about 96% sequence identity to SEQ ID NO: 43. The CAR can comprise a sequence having about 97% sequence identity to SEQ ID NO: 43. The CAR can comprise a sequence having about 98% sequence identity to SEQ ID NO: 43. The CAR can comprise a sequence having about 99% sequence identity to SEQ ID NO: 43. The CAR can comprise SEQ ID NO: 43. The CAR can consist of SEQ ID NO: 43.

In polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSPTTTPAPRPPTPAPTIASQPLSLRPEACRPAAGGAVHTRGLD FACDIYIWAPLAGTCGVLLLSLVITLYCNKRGRKKLLYIFKQPFMRPVQTTQEE DGCSCRFPEEEEGGCELRVKFSRSADAPAYQQGQNQLYNELNLGRREEYDVLD KRRGRDPEMGGKPRRKNPQEGLYNELQKDKMAEAYSEIGMKGERRRGKGHD GLYQGLSTATKDTYDALHMQALPPR (SEQ ID NO: 65). The CAR can comprise a sequence having about 85% sequence identity to SEQ ID NO: 65. The CAR can comprise a sequence having about 90% sequence identity to SEQ ID NO: 65. The CAR can comprise a sequence having about 95% sequence identity to SEQ ID NO: 65. The CAR can comprise a sequence having about 96% sequence identity to SEQ ID NO: 65. The CAR can comprise a sequence having about 97% sequence identity to SEQ ID NO: 65. The CAR can comprise a sequence having about 98% sequence identity to SEQ ID NO: 65. The CAR can comprise a sequence having about 99% sequence identity to SEQ ID NO: 65. The CAR can comprise SEQ ID NO: 65. The CAR can consist of SEQ ID NO: 65.

In polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSTTTPAPRPPTPAPTIASQPLSLRPEACRPAAGGAVHTRGLDF ACDIYIWAPLAGTCGVLLLSLVITLYCKRGRKKLLYIFKQPFMRPVQTTQEEDG CSCRFPEEEEGGCELRVKFSRSADAPAYQQGQNQLYNELNLGRREEYDVLDKR RGRDPEMGGKPRRKNPQEGLYNELQKDKMAEAYSEIGMKGERRRGKGHDGL YQGLSTATKDTYDALHMQALPPR (SEQ ID NO: 66). The CAR can comprise a sequence having about 85% sequence identity to SEQ ID NO: 66. The CAR can comprise a sequence having about 90% sequence identity to SEQ ID NO: 66. The CAR can comprise a sequence having about 95% sequence identity to SEQ ID NO: 66. The CAR can comprise a sequence having about 96% sequence identity to SEQ ID NO: 66. The CAR can comprise a sequence having about 97% sequence identity to SEQ ID NO: 66. The CAR can comprise a sequence having about 98% sequence identity to SEQ ID NO: 66. The CAR can comprise a sequence having about 99% sequence identity to SEQ ID NO: 66. The CAR can comprise SEQ ID NO: 66. The CAR can consist of SEQ ID NO: 66.

In polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSPTTTPAPRPPTPAPTIASQPLSLRPEACRPAAGGAVHTRGLD FACDIYIWAPLAGTCGVLLLSLVITLYCKRGRKKLLYIFKQPFMRPVQTTQEED GCSCRFPEEEEGGCELRVKFSRSADAPAYQQGQNQLYNELNLGRREEYDVLDK RRGRDPEMGGKPRRKNPQEGLYNELQKDKMAEAYSEIGMKGERRRGKGHDG LYQGLSTATKDTYDALHMQALPPR (SEQ ID NO: 67). The CAR can comprise a sequence having about 85% sequence identity to SEQ ID NO: 67. The CAR can comprise a sequence having about 90% sequence identity to SEQ ID NO: 67. The CAR can comprise a sequence having about 95% sequence identity to SEQ ID NO: 67. The CAR can comprise a sequence having about 96% sequence identity to SEQ ID NO: 67. The CAR can comprise a sequence having about 97% sequence identity to SEQ ID NO: 67. The CAR can comprise a sequence having about 98% sequence identity to SEQ ID NO: 67. The CAR can comprise a sequence having about 99% sequence identity to SEQ ID NO: 67. The CAR can comprise SEQ ID NO: 67. The CAR can consist of SEQ ID NO: 67.

In polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSTTTPAPRPPTPAPTIASQPLSLRPEACRPAAGGAVHTRGLDF ACDIYIWAPLAGTCGVLLLSLVITLYCNKRGRKKLLYIFKQPFMRPVQTTQEED GCSCRFPEEEEGGCELRVKFSRSADAPAYQQGQNQLYNELNLGRREEYDVLDK RRGRDPEMGGKPRRKNPQEGLYNELQKDKMAEAYSEIGMKGERRRGKGHDG LYQGLSTATKDTYDALHMQALPPR (SEQ ID NO: 68). The CAR can comprise a sequence having about 85% sequence identity to SEQ ID NO: 68. The CAR can comprise a sequence having about 90% sequence identity to SEQ ID NO: 68. The CAR can comprise a sequence having about 95% sequence identity to SEQ ID NO: 68. The CAR can comprise a sequence having about 96% sequence identity to SEQ ID NO: 68. The CAR can comprise a sequence having about 97% sequence identity to SEQ ID NO: 68. The CAR can comprise a sequence having about 98% sequence identity to SEQ ID NO: 68. The CAR can comprise a sequence having about 99% sequence identity to SEQ ID NO: 68. The CAR can comprise SEQ ID NO: 68. The CAR can consist of SEQ ID NO: 68.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] PTTTPAPRPPTPAPTIASQPLSLRPEACRPAAGGAVHTR GLDFACDIYIWAPLAGTCGVLLLSLVITLYCNKRGRKKLLYIFKQPFMRPVQTT QEEDGCSCRFPEEEEGGCELRVKFSRSADAPAYQQGQNQLYNELNLGRREEYD VLDKRRGRDPEMGGKPRRKNPQEGLYNELQKDKMAEAYSEIGMKGERRRGK GHDGLYQGLSTATKDTYDALHMQALPPR (SEQ ID NO: 69). The CAR can comprise a sequence having about 85% sequence identity to SEQ ID NO: 69. The CAR can comprise a sequence having about 90% sequence identity to SEQ ID NO: 69. The CAR can comprise a sequence having about 95% sequence identity to SEQ ID NO: 69. The CAR can comprise a sequence having about 96% sequence identity to SEQ ID NO: 69. The CAR can comprise a sequence having about 97% sequence identity to SEQ ID NO: 69. The CAR can comprise a sequence having about 98% sequence identity to SEQ ID NO: 69. The CAR can comprise a sequence having about 99% sequence identity to SEQ ID NO: 69. The CAR can comprise SEQ ID NO: 69. The CAR can consist of SEQ ID NO: 69.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] TTTPAPRPPTPAPTIASQPLSLRPEACRPAAGGAVHTRG LDFACDIYIWAPLAGTCGVLLLSLVITLYCNKRGRKKLLYIFKQPFMRPVQTTQ EEDGCSCRFPEEEEGGCELRVKFSRSADAPAYQQGQNQLYNELNLGRREEYDV LDKRRGRDPEMGGKPRRKNPQEGLYNELQKDKMAEAYSEIGMKGERRRGKG HDGLYQGLSTATKDTYDALHMQALPPR (SEQ ID NO: 70). The CAR can comprise a sequence having about 85% sequence identity to SEQ ID NO: 70. The CAR can comprise a sequence having about 90% sequence identity to SEQ ID NO: 70. The CAR can comprise a sequence having about 95% sequence identity to SEQ ID NO: 70. The CAR can comprise a sequence having about 96% sequence identity to SEQ ID NO: 70. The CAR can comprise a sequence having about 97% sequence identity to SEQ ID NO: 70. The CAR can comprise a sequence having about 98% sequence identity to SEQ ID NO: 70. The CAR can comprise a sequence having about 99% sequence identity to SEQ ID NO: 70. The CAR can comprise SEQ ID NO: 70. The CAR can consist of SEQ ID NO: 70.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] PTTTPAPRPPTPAPTIASQPLSLRPEACRPAAGGAVHTR GLDFACDIYIWAPLAGTCGVLLLSLVITLYCKRGRKKLLYIFKQPFMRPVQTTQ EEDGCSCRFPEEEEGGCELRVKFSRSADAPAYQQGQNQLYNELNLGRREEYDV LDKRRGRDPEMGGKPRRKNPQEGLYNELQKDKMAEAYSEIGMKGERRRGKG HDGLYQGLSTATKDTYDALHMQALPPR (SEQ ID NO: 71). The CAR can comprise a sequence having about 85% sequence identity to SEQ ID NO: 71. The CAR can comprise a sequence having about 90% sequence identity to SEQ ID NO: 71. The CAR can comprise a sequence having about 95% sequence identity to SEQ ID NO: 71. The CAR can comprise a sequence having about 96% sequence identity to SEQ ID NO: 71. The CAR can comprise a sequence having about 97% sequence identity to SEQ ID NO: 71. The CAR can comprise a sequence having about 98% sequence identity to SEQ ID NO: 71. The CAR can comprise a sequence having about 99% sequence identity to SEQ ID NO: 71. The CAR can comprise SEQ ID NO: 71. The CAR can consist of SEQ ID NO: 71.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] TTTPAPRPPTPAPTIASQPLSLRPEACRPAAGGAVHTR GLDFACDIYIWAPLAGTCGVLLLSLVITLYCKRGRKKLLYIFKQPFMRPVQTTQ EEDGCSCRFPEEEEGGCELRVKFSRSADAPAYQQGQNQLYNELNLGRREEYDV LDKRRGRDPEMGGKPRRKNPQEGLYNELQKDKMAEAYSEIGMKGERRRGKG HDGLYQGLSTATKDTYDALHMQALPPR (SEQ ID NO: 72). The CAR can comprise a sequence having about 85% sequence identity to SEQ ID NO: 72. The CAR can comprise a sequence having about 90% sequence identity to SEQ ID NO: 72. The CAR can comprise a sequence having about 95% sequence identity to SEQ ID NO: 72. The CAR can comprise a sequence having about 96% sequence identity to SEQ ID NO: 72. The CAR can comprise a sequence having about 97% sequence identity to SEQ ID NO: 72. The CAR can comprise a sequence having about 98% sequence identity to SEQ ID NO: 72. The CAR can comprise a sequence having about 99% sequence identity to SEQ ID NO: 72. The CAR can comprise SEQ ID NO: 72. The CAR can consist of SEQ ID NO: 72.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] PTTTPAPRPPTPAPTIASQPLSLRPEACRPAAGGAVHT RGLDFACDIYIWAPLAGTCGVLLLSLVITLYCKRGRKKLLYIFKQPFMRPVQTT QEEDGCSCRFPEEEEGGCELRVKFSRSADAPAYQQGQNQLYNELNLGRREEYD VLDKRRGRDPEMGGKPRRKNPQEGLYNELQKDKMAEAYSEIGMKGERRRGK GHDGLYQGLSTATKDTYDALHMQALPPR (SEQ ID NO: 73). The CAR can comprise a sequence having about 85% sequence identity to SEQ ID NO: 73. The CAR can comprise a sequence having about 90% sequence identity to SEQ ID NO: 73. The CAR can comprise a sequence having about 95% sequence identity to SEQ ID NO: 73. The CAR can comprise a sequence having about 96% sequence identity to SEQ ID NO: 73. The CAR can comprise a sequence having about 97% sequence identity to SEQ ID NO: 73. The CAR can comprise a sequence having about 98% sequence identity to SEQ ID NO: 73. The CAR can comprise a sequence having about 99% sequence identity to SEQ ID NO: 73. The CAR can comprise SEQ ID NO: 73. The CAR can consist of SEQ ID NO: 73.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] TTTPAPRPPTPAPTIASQPLSLRPEACRPAAGGAVHTR GLDFACDIYIWAPLAGTCGVLLLSLVITLYCNKRGRKKLLYIFKQPFMRPVQTT QEEDGCSCRFPEEEEGGCELRVKFSRSADAPAYQQGQNQLYNELNLGRREEYD VLDKRRGRDPEMGGKPRRKNPQEGLYNELQKDKMAEAYSEIGMKGERRRGK GHDGLYQGLSTATKDTYDALHMQALPPR (SEQ ID NO: 74). The CAR can comprise a sequence having about 85% sequence identity to SEQ ID NO: 74. The CAR can comprise a sequence having about 90% sequence identity to SEQ ID NO: 74. The CAR can comprise a sequence having about 95% sequence identity to SEQ ID NO: 74. The CAR can comprise a sequence having about 96% sequence identity to SEQ ID NO: 74. The CAR can comprise a sequence having about 97% sequence identity to SEQ ID NO: 74. The CAR can comprise a sequence having about 98% sequence identity to SEQ ID NO: 74. The CAR can comprise a sequence having about 99% sequence identity to SEQ ID NO: 74. The CAR can comprise SEQ ID NO: 74. The CAR can consist of SEQ ID NO: 74.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] TTTPAPRPPTPAPTIASQPLSLRPEACRPAAGGAVHTRG LDFACDIYIWAPLAGTCGVLLLSLVITLYCKRGRKKLLYIFKQPFMRPVQTTQEE DGCCRFPEEEEGGCELRVKFSRSADAPAYQQGQNQLYNELNLGRREEYDVLD KRRGRDPEMGGKPRRKNPQEGLYNELQKDKMAEAYSEIGMKGERRRGKGHD GLYQGLSTATKDTYDALHMQALPPR (SEQ ID NO: 75). The CAR can comprise a sequence having about 85% sequence identity to SEQ ID NO: 75. The CAR can comprise a sequence having about 90% sequence identity to SEQ ID NO: 75. The CAR can comprise a sequence having about 95% sequence identity to SEQ ID NO: 75. The CAR can comprise a sequence having about 96% sequence identity to SEQ ID NO: 75. The CAR can comprise a sequence having about 97% sequence identity to SEQ ID NO: 75. The CAR can comprise a sequence having about 98% sequence identity to SEQ ID NO: 75. The CAR can comprise a sequence having about 99% sequence identity to SEQ ID NO: 75. The CAR can comprise SEQ ID NO: 43. The CAR can consist of SEQ ID NO: 75.

The polynucleotide encoding a CAR described herein can comprise a nucleic acid sequence having about 80%, 85%, 90%, 95%, 96%, 97%, 98%, 99%, or 100% sequence identity to ATGGACTGGACATGGCGGATACTCTTCCTCGTCGCTGCTGCAACCGGAGCCC ACAGCCAGGTGCAGCTCCAGCAGTCTGGGCCAGGTTTGGTGACTCCTAGTC AGACATTGAGCTTGACTTGTGCTATCAGCGGAGACTCTGTTTCATCTAATTC TGCAACTTGGAACTGGATTCGGCAGTCCCCCAGCCGGGGGCTCGAGTGGTT GGGTCGGACCTACTATCGGAGCAAATGGTACAATGACTATGCAGTGAGCGT CAAATCAAGAATGAGCATCAATCCTGACACAAGCAAGAACCAGTTTAGCCT TCAGCTTAATAGCGTGACTCCAGAGGACACAGCTGTGTACTATTGCGCGAG AGGCATGATGACATACTATTACGGAATGGACGTGTGGGGCCAGGGAACTAC TGTTACAGTGTCAAGCGGAATCCTCGGTAGCGGAGGCGGCGGTTCCGGCGG AGGGGGTAGTGGTGGCGGGGGTAGTCAACCTGTGCTGACCCAGAGCAGCTC TCTTAGTGCTAGCCCAGGTGCAAGTGCAAGTCTTACCTGTACACTGCGCTCC GGTATTAATGTGGGCCCTTACCGAATTTACTGGTACCAGCAGAAACCAGGC TCCCCTCCCCAGTATCTGCTGAACTATAAGTCTGACTCAGACAAACAGCAGG GCTCCGGTGTGCCATCCCGATTTAGTGGCTCAAAGGATGCTAGTGCAAATGC CGGTGTTCTCCTGATCAGCGGACTCAGATCAGAGGACGAAGCAGACTATTA CTGTATGATTTGGCATAGCAGCGCTGCTGTCTTCGGAGGAGGGACTCAGCTC ACTGTCTTGAGTCGGGCCGCTGCACCTACCACTACCCCTGCCCCTCGACCCC CTACTCCCGCCCCAACTATCGCATCCCAACCACTCTCTCTCAGACCCGAAGC CTGTAGACCCGCAGCCGGTGGCGCTGTGCATACTCGCGGACTTGATTTTGCT TGTGATATTTATATCTGGGCCCCCCTTGCCGGAACTTGTGGAGTTCTCCTGCT GTCTCTCGTAATCACCCTTTATTGTAACAAACGGGGGCGCAAAAAACTTCTT TACATTTTCAAGCAGCCCTTTATGCGGCCCGTGCAGACCACACAGGAAGAA GATGGCTGCAGCTGCAGGTTCCCAGAGGAAGAAGAGGGCGGCTGCGAGCT GCGAGTAAAGTTCAGCCGGAGCGCCGATGCACCTGCATACCAGCAGGGTCA GAACCAGCTCTACAATGAGCTGAACCTGGGCAGAAGAGAGGAATATGATGT ACTCGACAAGAGAAGGGGACGCGATCCAGAGATGGGCGGCAAACCACGGC GGAAAAATCCGCAGGAGGGGCTCTATAACGAGCTCCAGAAGGACAAGATG GCAGAAGCCTACTCAGAAATTGGCATGAAAGGAGAGAGAAGGAGGGGAAA GGGCCATGATGGCCTTTACCAAGGGTTGTCTACTGCCACCAAGGATACGTA CGATGCACTCCATATGCAGGCTCTTCCTCCCCGA (SEQ ID NO: 50), ATGGACTGGACATGGCGGATACTCTTCCTCGTCGCTGCTGCAACCGGAGCCC ACAGCCAGGTGCAGCTCCAGCAGTCTGGGCCAGGTTTGGTGACTCCTAGTC AGACATTGAGCTTGACTTGTGCTATCAGCGGAGACTCTGTTTCATCTAATTC TGCAACTTGGAACTGGATTCGGCAGTCCCCCAGCCGGGGGCTCGAGTGGTT GGGTCGGACCTACTATCGGAGCAAATGGTACAATGACTATGCAGTGAGCGT CAAATCAAGAATGAGCATCAATCCTGACACAAGCAAGAACCAGTTTAGCCT TCAGCTTAATAGCGTGACTCCAGAGGACACAGCTGTGTACTATTGCGCGAG AGGCATGATGACATACTATTACGGAATGGACGTGTGGGGCCAGGGAACTAC TGTTACAGTGTCAAGCGGAATCCTCGGTAGCGGAGGCGGCGGTTCCGGCGG AGGGGGTAGTGGTGGCGGGGGTAGTCAACCTGTGCTGACCCAGAGCAGCTC TCTTAGTGCTAGCCCAGGTGCAAGTGCAAGTCTTACCTGTACACTGCGCTCC GGTATTAATGTGGGCCCTTACCGAATTTACTGGTACCAGCAGAAACCAGGA TCCCCTCCCCAGTATCTGCTGAACTATAAGTCTGACTCAGACAAACAGCAGG GCTCCGGTGTGCCATCCCGATTTAGTGGCTCAAAGGATGCTAGTGCAAATGC CGGTGTTCTCCTGATCAGCGGACTCAGATCAGAGGACGAAGCAGACTATTA CTGTATGATTTGGCATAGCAGCGCTGCTGTCTTCGGAGGAGGGACTCAGCTC ACTGTCTTGAGTCGGGCCGCTGCACCTACCACTACCCCTGCCCCTCGACCCC CTACTCCCGCCCCAACTATCGCATCCCAACCACTCTCTCTCAGACCCGAAGC CTGTAGACCCGCAGCCGGTGGCGCTGTGCATACTCGCGGACTTGATTTTGCT TGTGATATTTATATCTGGGCCCCCCTTGCCGGAACTTGTGGAGTTCTCCTGCT GTCTCTCGTAATCACCCTTTATTGTAACAAACGGGGGCGCAAAAAACTTCTT TACATTTTCAAGCAGCCCTTTATGCGGCCCGTGCAGACCACACAGGAAGAA GATGGCTGCAGCTGCAGGTTCCCAGAGGAAGAAGAGGGCGGCTGCGAGCT GCGAGTAAAGTTCAGCCGGAGCGCCGATGCACCTGCATACCAGCAGGGTCA GAACCAGCTCTACAATGAGCTGAACCTGGGCAGAAGAGAGGAATATGATGT ACTCGACAAGAGAAGGGGACGCGATCCAGAGATGGGCGGCAAACCACGGC GGAAAAATCCGCAGGAGGGGCTCTATAACGAGCTCCAGAAGGACAAGATG GCAGAAGCCTACTCAGAAATTGGCATGAAAGGAGAGAGAAGGAGGGGAAA GGGCCATGATGGCCTTTACCAAGGGTTGTCTACTGCCACCAAGGATACGTA CGATGCACTCCATATGCAGGCTCTTCCTCCCCGA (SEQ ID NO: 51), or ATGGATTGGACCTGGCGAATACTCTTCCTCGTCGCAGCGGCCACTGGTGCCC ATTCACAAGTCCAACTGCAGCAGAGCGGACCTGGCCTGGTGACACCCAGTC AGACTCTCAGCCTGACTTGTGCAATCAGCGGCGATAGTGTGTCTAGTAATTC TGCAACATGGAACTGGATCAGACAATCACCAAGTCGGGGACTGGAGTGGCT CGGTAGAACCTATTATAGGTCCAAATGGTATAACGATTATGCAGTGTCCGTG AAGTCCCGAATGTCTATCAACCCTGATACTAGTAAGAATCAATTCAGTCTGC AGCTTAACAGCGTAACCCCCGAAGATACTGCTGTGTATTACTGTGCCCGGG GTATGATGACTTACTACTACGGAATGGATGTGTGGGGGCAGGGAACAACCG TTACTGTTTCATCCGGCATTCTCGGGAGCGGAGGCGGTGGAAGCGGTGGGG GAGGGTCCGGGGGAGGAGGATCTCAGCCTGTTCTTACTCAATCTTCTTCCCT CTCCGCCTCACCCGGGGCCTCCGCCTCACTGACCTGCACTCTGCGATCAGGC ATCAACGTTGGGCCTTATAGAATCTACTGGTACCAGCAAAAGCCTGGATCA CCGCCCCAGTACCTGCTGAACTATAAATCAGACTCAGACAAGCAGCAGGGC TCCGGCGTGCCGAGTCGATTTAGCGGGAGCAAGGACGCGTCTGCTAATGCC GGCGTGCTTCTCATCAGCGGGCTCCGCAGTGAGGATGAGGCAGATTACTAC TGCATGATTTGGCATAGCAGTGCAGCCGTATTTGGCGGAGGAACACAGCTG ACTGTCCTCTCTCGCGCCGCCGCTCCGACCACCACCCCTGCACCACGCCCAC CTACTCCTGCGCCAACCATTGCCAGCCAGCCTCTCTCTCTCCGACCCGAGGC CTGTAGACCTGCCGCTGGCGGTGCAGTTCATACTCGGGGTCTCGATTTCGCC TGCGACATCTACATCTGGGCACCACTGGCTGGCACTTGTGGCGTTTTGCTCC TGTCCCTGGTGATCACTCTCTACTGTAATAAGAGGGGGAGGAAGAAACTCC TGTATATTTTCAAACAACCCTTTATGCGCCCTGTCCAAACCACCCAGGAAGA AGATGGATGTAGTTGCAGATTCCCAGAAGAAGAGGAGGGTGGGTGTGAACT TAGGGTGAAGTTTAGTCGCAGTGCCGACGCTCCCGCTTACCAACAGGGTCA GAACCAACTCTACAATGAGCTGAATCTGGGGAGGCGCGAAGAATACGACGT TCTGGATAAAAGACGCGGCCGCGACCCCGAGATGGGCGGGAAACCGCGGA GAAAGAACCCACAGGAAGGATTGTACAATGAGCTCCAGAAAGATAAGATG GCAGAAGCCTACTCCGAGATCGGCATGAAGGGGGAGCGAAGGCGCGGGAA AGGACACGATGGGCTGTACCAGGGTCTTTCAACCGCGACAAAGGACACCTA TGATGCTCTCCATATGCAGGCCCTCCCGCCACGC (SEQ ID NO: 52). The polynucleotide can comprise a nucleic acid sequence having about 85% sequence identity to SEQ ID NO: 50, SEQ ID NO: 51 or SEQ ID NO: 52. The polynucleotide can comprise a nucleic acid sequence having about 90% sequence identity to SEQ ID NO: 50, SEQ ID NO: 51 or SEQ ID NO: 52. The polynucleotide can comprise a nucleic acid sequence having about 95% sequence identity to SEQ ID NO: 50, SEQ ID NO: 51 or SEQ ID NO: 52. The polynucleotide can comprise a nucleic acid sequence having about 96% sequence identity to SEQ ID NO: 50, SEQ ID NO: 51 or SEQ ID NO: 52. The polynucleotide can comprise a nucleic acid sequence having about 97% sequence identity to SEQ ID NO: 50, SEQ ID NO: 51 or SEQ ID NO: 52. The polynucleotide can comprise a nucleic acid sequence having about 98% sequence identity to SEQ ID NO: 50, SEQ ID NO: 51 or SEQ ID NO: 52. The polynucleotide can comprise a nucleic acid sequence having about 99% sequence identity to SEQ ID NO: 50, SEQ ID NO: 51 or SEQ ID NO: 52. The polynucleotide can comprise SEQ ID NO: 50, SEQ ID NO: 51 or SEQ ID NO: 52. The polynucleotide can consists of SEQ ID NO: 50, SEQ ID NO: 51 or SEQ ID NO: 52.

In some embodiments, the polynucleotide encoding the CAR, the polynucleotide encoding IL-15, and optionally the polynucleotide encoding CCL19 are each independently transcribed under a promoter comprising a polynucleotide encoding a self-cleaving 2A peptide (2A peptide) or an internal ribosome entry site (IRES). In some embodiments, the polynucleotide encoding IL-15 and the polynucleotide encoding CCL19 are each independently transcribed under a promoter comprising a polynucleotide encoding the 2A peptide or IRES. In some embodiments, the polynucleotide encoding IL-15 is transcribed under a promoter comprising a polynucleotide encoding the 2A peptide or IRES.

In some embodiments, the polynucleotide encoding the CAR, the polynucleotide encoding IL-15, and optionally the polynucleotide encoding CCL19 are each independently transcribed under a promoter comprising a polynucleotide encoding the 2A peptide. There are four members in the 2A peptide family: P2A, E2A, F2A, and T2A. P2A is derived from porcine teschovirus-1 2A. E2A is derived from equine rhinitis A virus. F2A is derived from foot-and-mouth disease virus 18. T2A is derived from thosea asigna virus 2A. Exemplary sequences for 2A peptide members include:

P2A-
(SEQ ID NO: 77)
ATNFSLLKQAGDVEENPGP;
E2A-
(SEQ ID NO: 78)
QCTNYALLKLAGDVESNPGP;
F2A-
(SEQ ID NO: 79)
VKQTLNFDLLKLAGDVESNPGP;
and
T2A-
(SEQ ID NO: 80)
EGRGSLLTCGDVEENPGP.

In some embodiments, a peptide linker is further added to the terminus of the 2A peptide, e.g., at the N-terminus. In some embodiments, the peptide linker comprises GSG. In some cases, the P2A comprises GSGATNFSLLKQAGDVEENPGP (SEQ ID NO: 5).

In some embodiments, the polynucleotide encoding the CAR, the polynucleotide encoding IL-15, and optionally the polynucleotide encoding CCL19 are each independently transcribed under a promoter comprising a polynucleotide encoding a 2A peptide. In some instances, the polynucleotide encoding IL-15 and the polynucleotide encoding CCL19 are each transcribed under a promoter comprising the same 2A peptide. In some instances, the polynucleotide encoding IL-15 and the polynucleotide encoding CCL19 are each transcribed under a promoter comprising two different 2A peptides. In some cases, the polynucleotide encoding the CAR, the polynucleotide encoding IL-15, and the polynucleotide encoding CCL19 are each independently transcribed under a promoter comprising different 2A peptides.

In some embodiments, the polynucleotide encoding the CAR, the polynucleotide encoding IL-15, and optionally the polynucleotide encoding CCL19 are each independently transcribed under a promoter comprising a polynucleotide encoding the P2A peptide. The P2A peptide can comprise ATNFSLLKQAGDVEENPGP (SEQ ID NO: 77). The P2A peptide can also comprise SEQ ID NO: 5.

In some embodiments, the polynucleotide encoding the CAR and the polynucleotide encoding IL-15 are arranged in the nucleic acid molecule from the 5′ terminus to the 3′ terminus as:
the polynucleotide encoding the CAR—the polynucleotide encoding IL-15; or
the polynucleotide encoding IL-15—the polynucleotide encoding the CAR.

In some embodiments, the polynucleotide encoding the CAR, the polynucleotide encoding IL-15, and the polynucleotide encoding CCL19 are arranged in the nucleic acid molecule from the 5′ terminus to the 3′ terminus as:
the polynucleotide encoding the CAR—the polynucleotide encoding IL-15—the polynucleotide encoding CCL19;
the polynucleotide encoding the CAR—the polynucleotide encoding CCL19—the polynucleotide encoding IL-15;
the polynucleotide encoding IL-15—the polynucleotide encoding the CAR—the polynucleotide encoding CCL19;
the polynucleotide encoding CCL19—the polynucleotide encoding the CAR—the polynucleotide encoding IL-15;
the polynucleotide encoding IL-15—the polynucleotide encoding CCL19—the polynucleotide encoding the CAR; or
the polynucleotide encoding CCL19—the polynucleotide encoding IL-15—the polynucleotide encoding the CAR.

In some embodiments, the isolated nucleic acid molecule comprises a polynucleotide encoding mIL-15/Ra and a polynucleotide encoding CCL19. In some instances, the isolated nucleic acid molecule encodes a polypeptide comprising about 80%, 85%, 90%, 95%, 96%, 97%, 98%, 99%, or 100% sequence identity to MYRMQLLSCIALSLALVTNSATSNWVNVISDLKKIEDLIQSMHIDATLYTESDV HPSCKVTAMKCFLLELQVISLESGDASIHDTVENLIILANNSLSSNGNVTESGCK ECEELEEKNIKEFLQSFVHIVQMFINTSSGGGSGGGGSGGGGSGGGGSGGGSLQI TCPPPMSVEHADIWVKSYSLYSRERYICNSGFKRKAGTSSLTECVLNKATNVAH WTTPSLKCIRDPAL VHQRPAPPSTVTTAGVTPQPESLSPSGKEPAASSPSSNNTA ATTAAIVPGSQLMPSKSPSTGTTEISSHESSHGTPSQTTAKNWELTASASHQPPG VYPQGHSDTTVAISTSTVLLCGLSAVSLLACYLKSRQTPPLASVEMEAMEALPV TWGTSSRDEDLENCSHHLGSGATNFSLLKQAGDVEENPGPMALLLALSLLVLW TSPAPTLSGTNDAEDCCLSVTQKPIPGYIVRNFHYLLIKDGCRVPAVVFTTLRGR QLCAPPDQPWVERIIQRLQRTSAKMKRRSS (SEQ ID NO: 14) (also referred to herein as mIL15/Ra-P2A-CCL19). In some cases, the isolated nucleic acid molecule encodes a polypeptide comprising about 85% sequence identity to SEQ ID NO: 14. In some cases, the isolated nucleic acid molecule encodes a polypeptide comprising about 90% sequence identity to SEQ ID NO: 14. In some cases, the isolated nucleic acid molecule encodes a polypeptide comprising about 95% sequence identity to SEQ ID NO: 14. In some cases, the isolated nucleic acid molecule encodes a polypeptide comprising about 96% sequence identity to SEQ ID NO: 14. In some cases, the isolated nucleic acid molecule encodes a polypeptide comprising about 97% sequence identity to SEQ ID NO: 14. In some cases, the isolated nucleic acid molecule encodes a polypeptide comprising about 98% sequence identity to SEQ ID NO: 14. In some cases, the isolated nucleic acid molecule encodes a polypeptide comprising about 99% sequence identity to SEQ ID NO: 14. In some cases, the isolated nucleic acid molecule encodes a polypeptide comprising SEQ ID NO: 14. In some cases, the isolated nucleic acid molecule encodes a polypeptide consisting of SEQ ID NO: 14.

In some embodiments, the isolated nucleic acid molecule comprises a polynucleotide encoding sushi15 and a polynucleotide encoding CCL19. In some instances, the isolated nucleic acid molecule encodes a polypeptide comprising about 80%, 85%, 90%, 95%, 96%, 97%, 98%, 99%, or 100% sequence identity to MAPRRARGCRTLGLPALLLLLLLRPPATRGITCPPPMSVEHADIWVKSYSLYSR ERYICNSGFKRKAGTSSLTECVLNKATNVAHWTTPSLKCIRSGGGSGGGGSGGG GSGGGGSGGGSLONWVNVISDLKKIEDLIQSMHIDATLYTESDVHPSCKVTAMK CFLLELQVISLESGDASIHDTVENLIILANNSLSSNGNVTESGCKECEELEEKNIK EFLQSFVHIVQMFINTSGSGATNFSLLKQAGDVEENPGPMALLLALSLLVLWTS PAPTLSGTNDAEDCCLSVTQKPIPGYIVRNFHYLLIKDGCRVPAVVFTTLRGRQL CAPPDQPWVERIIQRLQRTSAKMKRRSS (SEQ ID NO: 31) (also referred to herein as IL15Rasp-sushi15-P2A-endosp-CCL19 or sushi15-CCL19). In some cases, the isolated nucleic acid molecule encodes a polypeptide comprising about 85% sequence identity to SEQ ID NO: 31. In some cases, the isolated nucleic acid molecule encodes a polypeptide comprising about 90% sequence identity to SEQ ID NO: 31. In some cases, the isolated nucleic acid molecule encodes a polypeptide comprising about 95% sequence identity to SEQ ID NO: 31. In some cases, the isolated nucleic acid molecule encodes a polypeptide comprising about 96% sequence identity to SEQ ID NO: 31. In some cases, the isolated nucleic acid molecule encodes a polypeptide comprising about 97% sequence identity to SEQ ID NO: 31. In some cases, the isolated nucleic acid molecule encodes a polypeptide comprising about 98% sequence identity to SEQ ID NO: 31. In some cases, the isolated nucleic acid molecule encodes a polypeptide comprising about 99% sequence identity to SEQ ID NO: 31. In some cases, the isolated nucleic acid molecule encodes a polypeptide comprising SEQ ID NO: 31. In some cases, the isolated nucleic acid molecule encodes a polypeptide consisting of SEQ ID NO: 31.

In some embodiments, the isolated nucleic acid molecule comprises a polynucleotide encoding a CAR that specifically binds to mesothelin and a polynucleotide encoding mIL-15/Ra. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSSGILGSGGGGSGGGGS GGGGSQPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSRAAAPTTTPAPRPPTPAPTIASQPLSLRPEACRPAAGGAVHT RGLDFACDIYIWAPLAGTCGVLLLSLVITLYCNKRGRKKLLYIFKQPFMRPVQT TQEEDGCSCRFPEEEEGGCELRVKFSRSADAPAYQQGQNQLYNELNLGRREEY DVLDKRRGRDPEMGGKPRRKNPQEGLYNELQKDKMAEAYSEIGMKGERRRG KGHDGLYQGLSTATKDTYDALHMQALPPRGSGATNFSLLKQAGDVEENPGPM YRMQLLSCIALSLALVTNSATSNWVNVISDLKKIEDLIQSMHIDATLYTESDVHP SCKVTAMKCFLLELQVISLESGDASIHDTVENLIILANNSLSSNGNVTESGCKEC EELEEKNIKEFLQSFVHIVQMFINTSSGGGSGGGGSGGGGSGGGGSGGGSLQITC PPPMSVEHADIWVKSYSLYSRERYICNSGFKRKAGTSSLTECVLNKATNVAHW TTPSLKCIRDPALVHQRPAPPSTVTTAGVTPQPESLSPSGKEPAASSPSSNNTAAT TAAIVPGSQLMPSKSPSTGTTEISSHESSHGTPSQTTAKNWELTASASHQPPGVY PQGHSDTTVAISTSTVLLCGLSAVSLLACYLKSRQTPPLASVEMEAMEALPVTW GTSSRDEDLENCSHHL (SEQ ID NO: 34) (also referred to herein as ssV_{H}-P4-CAR-P2A-IL2sp-mIL15/Ra or P4-BB-mIL15/Ra). In some cases, the isolated nucleic acid molecule encodes a polypeptide comprising about 85% sequence identity to SEQ ID NO: 34. In some cases, the isolated nucleic acid molecule encodes a polypeptide comprising about 90% sequence identity to SEQ ID NO: 34. In some cases, the isolated nucleic acid molecule encodes a polypeptide comprising about 95% sequence identity to SEQ ID NO: 34. In some cases, the isolated nucleic acid molecule encodes a polypeptide comprising about 96% sequence identity to SEQ ID NO: 34. In some cases, the isolated nucleic acid molecule encodes a polypeptide comprising about 97% sequence identity to SEQ ID NO: 34. In some cases, the isolated nucleic acid molecule encodes a polypeptide comprising about 98% sequence identity to SEQ ID NO: 34. In some cases, the isolated nucleic acid molecule encodes a polypeptide comprising about 99% sequence identity to SEQ ID NO: 34. In some cases, the isolated nucleic acid molecule encodes a polypeptide comprising SEQ ID NO: 34. In some cases, the isolated nucleic acid molecule encodes a polypeptide consisting of SEQ ID NO: 34.

In some embodiments, the isolated nucleic acid molecule encoding ssV_{H}-P4-CAR-P2A-IL2sp-mIL15/Ra or P4-BB-mIL15/Ra comprises a sequence comprising about 80%, 85%, 90%, 95%, 96%, 97%, 98%, 99%, or 100% sequence identity to ATGGACTGGACATGGCGGATACTCTTCCTCGTCGCTGCTGCAACCGGAGCCC ACAGCCAGGTGCAGCTCCAGCAGTCTGGGCCAGGTTTGGTGACTCCTAGTC AGACATTGAGCTTGACTTGTGCTATCAGCGGAGACTCTGTTTCATCTAATTC TGCAACTTGGAACTGGATTCGGCAGTCCCCCAGCCGGGGGCTCGAGTGGTT GGGTCGGACCTACTATCGGAGCAAATGGTACAATGACTATGCAGTGAGCGT CAAATCAAGAATGAGCATCAATCCTGACACAAGCAAGAACCAGTTTAGCCT TCAGCTTAATAGCGTGACTCCAGAGGACACAGCTGTGTACTATTGCGCGAG AGGCATGATGACATACTATTACGGAATGGACGTGTGGGGCCAGGGAACTAC TGTTACAGTGTCAAGCGGAATCCTCGGTAGCGGAGGCGGCGGTTCCGGCGG AGGGGGTAGTGGTGGCGGGGGTAGTCAACCTGTGCTGACCCAGAGCAGCTC TCTTAGTGCTAGCCCAGGTGCAAGTGCAAGTCTTACCTGTACACTGCGCTCC GGTATTAATGTGGGCCCTTACCGAATTTACTGGTACCAGCAGAAACCAGGC TCCCCTCCCCAGTATCTGCTGAACTATAAGTCTGACTCAGACAAACAGCAGG GCTCCGGTGTGCCATCCCGATTTAGTGGCTCAAAGGATGCTAGTGCAAATGC CGGTGTTCTCCTGATCAGCGGACTCAGATCAGAGGACGAAGCAGACTATTA CTGTATGATTTGGCATAGCAGCGCTGCTGTCTTCGGAGGAGGGACTCAGCTC ACTGTCTTGAGTCGGGCCGCTGCACCTACCACTACCCCTGCCCCTCGACCCC CTACTCCCGCCCCAACTATCGCATCCCAACCACTCTCTCTCAGACCCGAAGC CTGTAGACCCGCAGCCGGTGGCGCTGTGCATACTCGCGGACTTGATTTTGCT TGTGATATTTATATCTGGGCCCCCCTTGCCGGAACTTGTGGAGTTCTCCTGCT GTCTCTCGTAATCACCCTTTATTGTAACAAACGGGGGCGCAAAAAACTTCTT TACATTTTCAAGCAGCCCTTTATGCGGCCCGTGCAGACCACACAGGAAGAA GATGGCTGCAGCTGCAGGTTCCCAGAGGAAGAAGAGGGCGGCTGCGAGCT GCGAGTAAAGTTCAGCCGGAGCGCCGATGCACCTGCATACCAGCAGGGTCA GAACCAGCTCTACAATGAGCTGAACCTGGGCAGAAGAGAGGAATATGATGT ACTCGACAAGAGAAGGGGACGCGATCCAGAGATGGGCGGCAAACCACGGC GGAAAAATCCGCAGGAGGGGCTCTATAACGAGCTCCAGAAGGACAAGATG GCAGAAGCCTACTCAGAAATTGGCATGAAAGGAGAGAGAAGGAGGGGAAA GGGCCATGATGGCCTTTACCAAGGGTTGTCTACTGCCACCAAGGATACGTA CGATGCACTCCATATGCAGGCTCTTCCTCCCCGAGGTTCAGGCGCAACAAAT TTTTCACTTCTTAAACAAGCTGGCGATGTCGAGGAAAACCCAGGTCCCATGT ATAGAATGCAGCTTCTGTCATGTATCGCACTGAGTCTGGCCCTGGTGACCAA CAGTGCCACCAGCAACTGGGTGAATGTGATAAGCGACCTTAAGAAAATAGA AGACCTTATTCAGTCCATGCACATAGATGCCACACTGTACACCGAGAGCGA TGTGCACCCTTCCTGCAAAGTGACAGCTATGAAATGCTTCCTTCTGGAACTG CAAGTAATTTCATTGGAATCTGGCGATGCTTCCATACATGACACCGTGGAAA ACCTTATTATTTTGGCTAACAATTCATTGAGCTCAAATGGAAACGTGACAGA ATCCGGTTGTAAGGAATGTGAAGAGCTGGAAGAAAAAAATATCAAGGAATT CCTGCAGAGCTTTGTTCACATTGTGCAAATGTTTATTAATACATCCTCAGGG GGCGGTTCCGGAGGCGGGGGAAGTGGCGGAGGAGGAAGCGGCGGAGGAGG AAGCGGAGGAGGATCACTTCAAATCACATGTCCCCCCCCTATGAGTGTTGA ACATGCTGACATCTGGGTGAAATCCTATTCCCTTTATTCAAGAGAACGATAC ATATGTAATTCCGGGTTTAAGAGGAAAGCAGGCACATCATCTCTCACCGAA TGTGTCCTGAATAAGGCGACAAACGTAGCTCACTGGACTACGCCCTCCCTCA AATGCATTAGAGACCCAGCACTCGTGCACCAAAGGCCAGCCCCCCCAAGCA CCGTCACTACTGCAGGTGTAACCCCGCAACCAGAATCCCTCTCACCAAGCG GAAAAGAGCCAGCCGCATCTTCTCCTAGTTCCAATAATACAGCCGCGACAA CAGCCGCAATTGTCCCTGGAAGCCAGTTGATGCCATCAAAGTCCCCAAGTA CGGGTACGACCGAAATCTCCTCCCACGAAAGCAGCCACGGAACACCAAGCC AGACTACCGCCAAGAACTGGGAGCTGACCGCTTCTGCATCACATCAGCCGC CGGGAGTGTATCCACAGGGGCACTCTGATACCACAGTAGCAATCTCAACCT CCACCGTCCTGCTGTGTGGCCTTAGCGCTGTGTCTCTCCTCGCATGTTACCTC AAATCCAGGCAGACCCCCCCCCTTGCTAGTGTCGAAATGGAGGCAATGGAA GCACTTCCCGTGACATGGGGCACTTCTAGCAGAGATGAGGACCTTGAAAAC TGCTCACACCACCTC (SEQ ID NO: 37). In some cases, the isolated nucleic acid molecule comprises a sequence comprising about 85% sequence identity to SEQ ID NO: 37. In some cases, the isolated nucleic acid molecule comprises a sequence comprising about 90% sequence identity to SEQ ID NO: 37. In some cases, the isolated nucleic acid molecule comprises a sequence comprising about 95% sequence identity to SEQ ID NO: 37. In some cases, the isolated nucleic acid molecule comprises a sequence comprising about 96% sequence identity to SEQ ID NO: 37. In some cases, the isolated nucleic acid molecule comprises a sequence comprising about 97% sequence identity to SEQ ID NO: 37. In some cases, the isolated nucleic acid molecule comprises a sequence comprising about 98% sequence identity to SEQ ID NO: 37. In some cases, the isolated nucleic acid molecule comprises a sequence comprising about 99% sequence identity to SEQ ID NO: 37. In some cases, the isolated nucleic acid molecule comprises SEQ ID NO: 37. In some cases, the isolated nucleic acid molecule consists of SEQ ID NO: 37.

In some embodiments, the isolated nucleic acid molecule comprises a polynucleotide encoding a CAR that specifically binds to mesothelin and a polynucleotide encoding sushi15. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSSGILGSGGGGSGGGGS GGGGSQPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSRAAAPTTTPAPRPPTPAPTIASQPLSLRPEACRPAAGGAVHT RGLDFACDIYIWAPLAGTCGVLLLSLVITLYCNKRGRKKLLYIFKQPFMRPVQT TQEEDGCSCRFPEEEEGGCELRVKFSRSADAPAYQQGQNQLYNELNLGRREEY DVLDKRRGRDPEMGGKPRRKNPQEGLYNELQKDKMAEAYSEIGMKGERRRG KGHDGLYQGLSTATKDTYDALHMQALPPRGSGATNFSLLKQAGDVEENPGPM APRRARGCRTLGLPALLLLLLLRPPATRGITCPPPMSVEHADIWVKSYSLYSRER YICNSGFKRKAGTSSLTECVLNKATNVAHWTTPSLKCIRSGGGSGGGGSGGGG SGGGGSGGGSLQNWVNVISDLKKIEDLIQSMHIDATLYTESDVHPSCKVTAMK CFLLELQVISLESGDASIHDTVENLIILANNSLSSNGNVTESGCKECEELEEKNIK EFLQSFVHIVQMFINTS (SEQ ID NO: 35) (also referred to herein as ssV_{H}-P4-CAR-P2A-sushi15 or P4-BB-sushi15). In some cases, the isolated nucleic acid molecule encodes a polypeptide comprising about 85% sequence identity to SEQ ID NO: 35. In some cases, the isolated nucleic acid molecule encodes a polypeptide comprising about 90% sequence identity to SEQ ID NO: 35. In some cases, the isolated nucleic acid molecule encodes a polypeptide comprising about 95% sequence identity to SEQ ID NO: 35. In some cases, the isolated nucleic acid molecule encodes a polypeptide comprising about 96% sequence identity to SEQ ID NO: 35. In some cases, the isolated nucleic acid molecule encodes a polypeptide comprising about 97% sequence identity to SEQ ID NO: 35. In some cases, the isolated nucleic acid molecule encodes a polypeptide comprising about 98% sequence identity to SEQ ID NO: 35. In some cases, the isolated nucleic acid molecule encodes a polypeptide comprising about 99% sequence identity to SEQ ID NO: 35. In some cases, the isolated nucleic acid molecule encodes a polypeptide comprising SEQ ID NO: 35. In some cases, the isolated nucleic acid molecule encodes a polypeptide consisting of SEQ ID NO: 35.

In some embodiments, the isolated nucleic acid molecule encoding ssV_{H}-P4-CAR-P2A-sushi15 or P4-BB-sushi15 comprises a sequence comprising about 80%, 85%, 90%, 95%, 96%, 97%, 98%, 99%, or 100% sequence identity to ATGGACTGGACATGGCGGATACTCTTCCTCGTCGCTGCTGCAACCGGAGCCC ACAGCCAGGTGCAGCTCCAGCAGTCTGGGCCAGGTTTGGTGACTCCTAGTC AGACATTGAGCTTGACTTGTGCTATCAGCGGAGACTCTGTTTCATCTAATTC TGCAACTTGGAACTGGATTCGGCAGTCCCCCAGCCGGGGGCTCGAGTGGTT GGGTCGGACCTACTATCGGAGCAAATGGTACAATGACTATGCAGTGAGCGT CAAATCAAGAATGAGCATCAATCCTGACACAAGCAAGAACCAGTTTAGCCT TCAGCTTAATAGCGTGACTCCAGAGGACACAGCTGTGTACTATTGCGCGAG AGGCATGATGACATACTATTACGGAATGGACGTGTGGGGCCAGGGAACTAC TGTTACAGTGTCAAGCGGAATCCTCGGTAGCGGAGGCGGCGGTTCCGGCGG AGGGGGTAGTGGTGGCGGGGGTAGTCAACCTGTGCTGACCCAGAGCAGCTC TCTTAGTGCTAGCCCAGGTGCAAGTGCAAGTCTTACCTGTACACTGCGCTCC GGTATTAATGTGGGCCCTTACCGAATTTACTGGTACCAGCAGAAACCAGGA TCCCCTCCCCAGTATCTGCTGAACTATAAGTCTGACTCAGACAAACAGCAGG GCTCCGGTGTGCCATCCCGATTTAGTGGCTCAAAGGATGCTAGTGCAAATGC CGGTGTTCTCCTGATCAGCGGACTCAGATCAGAGGACGAAGCAGACTATTA CTGTATGATTTGGCATAGCAGCGCTGCTGTCTTCGGAGGAGGGACTCAGCTC ACTGTCTTGAGTCGGGCCGCTGCACCTACCACTACCCCTGCCCCTCGACCCC CTACTCCCGCCCCAACTATCGCATCCCAACCACTCTCTCTCAGACCCGAAGC CTGTAGACCCGCAGCCGGTGGCGCTGTGCATACTCGCGGACTTGATTTTGCT TGTGATATTTATATCTGGGCCCCCCTTGCCGGAACTTGTGGAGTTCTCCTGCT GTCTCTCGTAATCACCCTTTATTGTAACAAACGGGGGCGCAAAAAACTTCTT TACATTTTCAAGCAGCCCTTTATGCGGCCCGTGCAGACCACACAGGAAGAA GATGGCTGCAGCTGCAGGTTCCCAGAGGAAGAAGAGGGCGGCTGCGAGCT GCGAGTAAAGTTCAGCCGGAGCGCCGATGCACCTGCATACCAGCAGGGTCA GAACCAGCTCTACAATGAGCTGAACCTGGGCAGAAGAGAGGAATATGATGT ACTCGACAAGAGAAGGGGACGCGATCCAGAGATGGGCGGCAAACCACGGC GGAAAAATCCGCAGGAGGGGCTCTATAACGAGCTCCAGAAGGACAAGATG GCAGAAGCCTACTCAGAAATTGGCATGAAAGGAGAGAGAAGGAGGGGAAA GGGCCATGATGGCCTTTACCAAGGGTTGTCTACTGCCACCAAGGATACGTA CGATGCACTCCATATGCAGGCTCTTCCTCCCCGAGGGTCTGGCGCTACGAAT TTCTCTCTCCTTAAACAGGCCGGAGACGTGGAAGAAAATCCCGGCCCGATG GCGCCCCGCAGGGCCCGCGGGTGTCGAACATTGGGTCTGCCTGCTCTCTTGC TGCTGTTGCTTCTCAGACCTCCCGCCACACGCGGAATTACGTGCCCTCCCCC CATGTCTGTGGAACATGCCGACATATGGGTCAAGTCTTACAGTCTTTACTCT AGAGAACGGTATATCTGCAATAGCGGGTTCAAAAGAAAAGCAGGGACTTCC AGCCTGACAGAGTGCGTACTGAATAAGGCCACTAACGTTGCTCACTGGACC ACCCCATCATTGAAGTGTATTCGATCAGGAGGCGGAAGCGGTGGTGGGGGC TCAGGGGGTGGCGGTAGTGGAGGCGGGGGCAGCGGAGGGGGCTCTTTGCA AAACTGGGTTAATGTTATTAGCGACCTTAAGAAAATCGAGGACCTGATACA GTCCATGCACATCGATGCGACCCTGTACACTGAGAGCGATGTGCATCCCAG TTGCAAAGTGACTGCTATGAAATGCTTTCTGCTCGAGTTGCAGGTGATCTCC CTGGAAAGCGGCGACGCCTCAATACACGACACGGTCGAAAATCTGATCATT CTCGCCAACAACTCTCTCTCAAGTAACGGGAATGTGACAGAAAGTGGATGC AAAGAATGCGAGGAACTTGAGGAGAAAAACATTAAAGAATTCCTCCAGTCC TTCGTCCACATCGTGCAGATGTTTATCAATACTTCC (SEQ ID NO: 38). In some cases, the isolated nucleic acid molecule comprises a sequence comprising about 85% sequence identity to SEQ ID NO: 38. In some cases, the isolated nucleic acid molecule comprises a sequence comprising about 90% sequence identity to SEQ ID NO: 38. In some cases, the isolated nucleic acid molecule comprises a sequence comprising about 95% sequence identity to SEQ ID NO: 38. In some cases, the isolated nucleic acid molecule comprises a sequence comprising about 96% sequence identity to SEQ ID NO: 38. In some cases, the isolated nucleic acid molecule comprises a sequence comprising about 97% sequence identity to SEQ ID NO: 38. In some cases, the isolated nucleic acid molecule comprises a sequence comprising about 98% sequence identity to SEQ ID NO: 38. In some cases, the isolated nucleic acid molecule comprises a sequence comprising about 99% sequence identity to SEQ ID NO: 38. In some cases, the isolated nucleic acid molecule comprises SEQ ID NO: 38. In some cases, the isolated nucleic acid molecule consists of SEQ ID NO: 38.

In some embodiments, the isolated nucleic acid molecule comprises a polynucleotide encoding a CAR that specifically binds to mesothelin, a polynucleotide encoding mIL-15/Ra and a polynucleotide encoding CCL19. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSSGILGSGGGGSGGGGS GGGGSQPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSRAAAPTTTPAPRPPTPAPTIASQPLSLRPEACRPAAGGAVHT RGLDFACDIYIWAPLAGTCGVLLLSLVITLYCNKRGRKKLLYIFKQPFMRPVQT TQEEDGCSCRFPEEEEGGCELRVKFSRSADAPAYQQGQNQLYNELNLGRREEY DVLDKRRGRDPEMGGKPRRKNPQEGLYNELQKDKMAEAYSEIGMKGERRRG KGHDGLYQGLSTATKDTYDALHMQALPPRGSGATNFSLLKQAGDVEENPGPM YRMQLLSCIALSLALVTNSATSNWVNVISDLKKIEDLIQSMHIDATLYTESDVHP SCKVTAMKCFLLELQVISLESGDASIHDTVENLIILANNSLSSNGNVTESGCKEC EELEEKNIKEFLQSFVHIVQMFINTSSGGGSGGGGSGGGGSGGGGSGGGSLQITC PPPMSVEHADIWVKSYSLYSRERYICNSGFKRKAGTSSLTECVLNKATNVAHW TTPSLKCIRDPALVHQRPAPPSTVTTAGVTPQPESLSPSGKEPAASSPSSNNTAAT TAAIVPGSQLMPSKSPSTGTTEISSHESSHGTPSQTTAKNWELTASASHQPPGVY PQGHSDTTVAISTSTVLLCGLSAVSLLACYLKSRQTPPLASVEMEAMEALPVTW GTSSRDEDLENCSHHLGSGATNFSLLKQAGDVEENPGPMALLLALSLLVLWTS PAPTLSGTNDAEDCCLSVTQKPIPGYIVRNFHYLLIKDGCRVPAVVFTTLRGRQL CAPPDQPWVERIIQRLQRTSAKMKRRSS (SEQ ID NO: 36) (also referred to herein as ssV_{H}-P4-CAR-P2A-IL2sp-mIL15/Ra-P2A-CCL19 or P4-BB-mIL15-CCL19). In some cases, the isolated nucleic acid molecule encodes a polypeptide comprising about 85% sequence identity to SEQ ID NO: 36. In some cases, the isolated nucleic acid molecule encodes a polypeptide comprising about 90% sequence identity to SEQ ID NO: 36. In some cases, the isolated nucleic acid molecule encodes a polypeptide comprising about 95% sequence identity to SEQ ID NO: 36. In some cases, the isolated nucleic acid molecule encodes a polypeptide comprising about 96% sequence identity to SEQ ID NO: 36. In some cases, the isolated nucleic acid molecule encodes a polypeptide comprising about 97% sequence identity to SEQ ID NO: 36. In some cases, the isolated nucleic acid molecule encodes a polypeptide comprising about 98% sequence identity to SEQ ID NO: 36. In some cases, the isolated nucleic acid molecule encodes a polypeptide comprising about 99% sequence identity to SEQ ID NO: 36. In some cases, the isolated nucleic acid molecule encodes a polypeptide comprising SEQ ID NO: 36. In some cases, the isolated nucleic acid molecule encodes a polypeptide consisting of SEQ ID NO: 36.

In some embodiments, the isolated nucleic acid molecule encoding ssV_{H}-P4-CAR-P2A-IL2sp-mIL15/Ra-P2A-CCL19 or P4-BB-mIL15-CCL19 comprises a sequence comprising about 80%, 85%, 90%, 95%, 96%, 97%, 98%, 99%, or 100% sequence identity to ATGGATTGGACCTGGCGAATACTCTTCCTCGTCGCAGCGGCCACTGGTGCCC ATTCACAAGTCCAACTGCAGCAGAGCGGACCTGGCCTGGTGACACCCAGTC AGACTCTCAGCCTGACTTGTGCAATCAGCGGCGATAGTGTGTCTAGTAATTC TGCAACATGGAACTGGATCAGACAATCACCAAGTCGGGGACTGGAGTGGCT CGGTAGAACCTATTATAGGTCCAAATGGTATAACGATTATGCAGTGTCCGTG AAGTCCCGAATGTCTATCAACCCTGATACTAGTAAGAATCAATTCAGTCTGC AGCTTAACAGCGTAACCCCCGAAGATACTGCTGTGTATTACTGTGCCCGGG GTATGATGACTTACTACTACGGAATGGATGTGTGGGGGCAGGGAACAACCG TTACTGTTTCATCCGGCATTCTCGGGAGCGGAGGCGGTGGAAGCGGTGGGG GAGGGTCCGGGGGAGGAGGATCTCAGCCTGTTCTTACTCAATCTTCTTCCCT CTCCGCCTCACCCGGGGCCTCCGCCTCACTGACCTGCACTCTGCGATCAGGC ATCAACGTTGGGCCTTATAGAATCTACTGGTACCAGCAAAAGCCTGGATCA CCGCCCCAGTACCTGCTGAACTATAAATCAGACTCAGACAAGCAGCAGGGC TCCGGCGTGCCGAGTCGATTTAGCGGGAGCAAGGACGCGTCTGCTAATGCC GGCGTGCTTCTCATCAGCGGGCTCCGCAGTGAGGATGAGGCAGATTACTAC TGCATGATTTGGCATAGCAGTGCAGCCGTATTTGGCGGAGGAACACAGCTG ACTGTCCTCTCTCGCGCCGCCGCTCCGACCACCACCCCTGCACCACGCCCAC CTACTCCTGCGCCAACCATTGCCAGCCAGCCTCTCTCTCTCCGACCCGAGGC CTGTAGACCTGCCGCTGGCGGTGCAGTTCATACTCGGGGTCTCGATTTCGCC TGCGACATCTACATCTGGGCACCACTGGCTGGCACTTGTGGCGTTTTGCTCC TGTCCCTGGTGATCACTCTCTACTGTAATAAGAGGGGGAGGAAGAAACTCC TGTATATTTTCAAACAACCCTTTATGCGCCCTGTCCAAACCACCCAGGAAGA AGATGGATGTAGTTGCAGATTCCCAGAAGAAGAGGAGGGTGGGTGTGAACT TAGGGTGAAGTTTAGTCGCAGTGCCGACGCTCCCGCTTACCAACAGGGTCA GAACCAACTCTACAATGAGCTGAATCTGGGGAGGCGCGAAGAATACGACGT TCTGGATAAAAGACGCGGCCGCGACCCCGAGATGGGCGGGAAACCGCGGA GAAAGAACCCACAGGAAGGATTGTACAATGAGCTCCAGAAAGATAAGATG GCAGAAGCCTACTCCGAGATCGGCATGAAGGGGGAGCGAAGGCGCGGGAA AGGACACGATGGGCTGTACCAGGGTCTTTCAACCGCGACAAAGGACACCTA TGATGCTCTCCATATGCAGGCCCTCCCGCCACGCGGAAGTGGAGCAACTAA TTTTAGCCTTCTGAAACAAGCTGGCGATGTTGAGGAAAATCCTGGGCCGAT GTACAGGATGCAGCTGCTTTCTTGCATTGCACTGAGTTTGGCACTCGTCACC AACTCTGCCACATCAAATTGGGTTAACGTTATCAGCGATCTGAAGAAAATC GAGGATTTGATCCAGAGTATGCATATTGACGCAACCTTGTATACAGAATCTG ATGTGCACCCAAGCTGTAAAGTCACAGCTATGAAATGCTTTTTGCTGGAACT CCAAGTGATCTCCCTCGAATCCGGCGATGCATCCATCCACGATACTGTCGAA AACCTTATAATTTTGGCCAATAACAGCCTCAGCAGCAATGGCAACGTGACA GAGTCTGGGTGTAAGGAGTGTGAAGAACTGGAGGAGAAAAATATTAAAGA ATTCCTGCAGTCCTTTGTACACATTGTGCAAATGTTCATTAACACTTCAAGT GGCGGCGGGAGCGGCGGGGGTGGTTCAGGTGGTGGCGGCAGCGGTGGTGG GGGGTCTGGCGGGGGTAGTCTCCAAATTACTTGTCCTCCCCCAATGAGCGTT GAACACGCCGACATTTGGGTCAAGTCTTATTCACTGTACAGCCGAGAAAGA TATATCTGTAACTCTGGATTTAAGCGCAAGGCCGGAACGTCTAGTCTGACTG AGTGCGTGCTGAATAAGGCCACTAATGTTGCCCACTGGACTACCCCCAGCCT GAAGTGTATTCGCGATCCTGCCTTGGTGCACCAACGACCCGCGCCACCCAG CACAGTCACTACTGCCGGTGTGACTCCACAGCCCGAGTCTTTGTCCCCGAGC GGAAAGGAGCCCGCCGCATCTTCACCTTCTTCAAATAACACGGCCGCCACA ACCGCTGCAATCGTCCCAGGTAGTCAACTGATGCCCTCTAAAAGCCCCTCTA CGGGGACAACTGAGATAAGCAGCCACGAGTCTAGTCACGGCACACCAAGCC AGACTACCGCCAAAAACTGGGAGCTGACCGCCTCTGCCTCACACCAACCAC CAGGCGTGTATCCCCAGGGGCACAGCGACACCACTGTGGCAATCAGCACCA GCACGGTACTGTTGTGCGGACTCTCTGCCGTCAGTCTGCTGGCCTGCTACCT GAAATCCAGACAGACTCCCCCCCTGGCCAGCGTGGAAATGGAAGCTATGGA GGCTCTGCCCGTGACCTGGGGGACTAGCTCCAGAGATGAAGACTTGGAGAA CTGCAGTCACCATCTCGGGTCCGGAGCCACGAATTTCTCTCTCCTCAAACAA GCTGGGGATGTTGAGGAGAACCCTGGGCCAATGGCCCTCTTGCTCGCACTG TCCCTCCTGGTCCTGTGGACATCACCCGCCCCCACCCTGTCCGGCACGAATG ACGCAGAAGACTGCTGCCTGTCTGTCACGCAGAAACCCATCCCCGGCTATA TAGTGCGGAACTTCCATTACCTGCTGATCAAGGACGGATGTAGGGTGCCAG CCGTCGTCTTCACCACCCTGCGAGGGCGCCAGCTGTGCGCTCCTCCTGACCA GCCCTGGGTGGAGCGGATCATTCAACGCTTGCAGCGCACCTCAGCAAAAAT GAAAAGAAGAAGTAGT (SEQ ID NO: 39). In some cases, the isolated nucleic acid molecule comprises a sequence comprising about 85% sequence identity to SEQ ID NO: 39. In some cases, the isolated nucleic acid molecule comprises a sequence comprising about 90% sequence identity to SEQ ID NO: 39. In some cases, the isolated nucleic acid molecule comprises a sequence comprising about 95% sequence identity to SEQ ID NO: 39. In some cases, the isolated nucleic acid molecule comprises a sequence comprising about 96% sequence identity to SEQ ID NO: 39. In some cases, the isolated nucleic acid molecule comprises a sequence comprising about 97% sequence identity to SEQ ID NO: 39. In some cases, the isolated nucleic acid molecule comprises a sequence comprising about 98% sequence identity to SEQ ID NO: 39. In some cases, the isolated nucleic acid molecule comprises a sequence comprising about 99% sequence identity to SEQ ID NO: 39. In some cases, the isolated nucleic acid molecule comprises SEQ ID NO: 39. In some cases, the isolated nucleic acid molecule consists of SEQ ID NO: 39.

In some embodiments, the isolated nucleic acid molecule comprises a polynucleotide encoding a CAR that specifically binds to mesothelin, a polynucleotide encoding sushi15 and a polynucleotide encoding CCL19. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSSGILGSGGGGSGGGGS GGGGSQPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSRAAAPTTTPAPRPPTPAPTIASQPLSLRPEACRPAAGGAVHT RGLDFACDIYIWAPLAGTCGVLLLSLVITLYCNKRGRKKLLYIFKQPFMRPVQT TQEEDGCSCRFPEEEEGGCELRVKFSRSADAPAYQQGQNQLYNELNLGRREEY DVLDKRRGRDPEMGGKPRRKNPQEGLYNELQKDKMAEAYSEIGMKGERRRG KGHDGLYQGLSTATKDTYDALHMQALPPRGSGATNFSLLKQAGDVEENPGPM APRRARGCRTLGLPALLLLLLLRPPATRGITCPPPMSVEHADIWVKSYSLYSRER YICNSGFKRKAGTSSLTECVLNKATNVAHWTTPSLKCIRSGGGSGGGGSGGGG SGGGGSGGGSLQNWVNVISDLKKIEDLIQSMHIDATLYTESDVHPSCKVTAMK CFLLELQVISLESGDASIHDTVENLIILANNSLSSNGNVTESGCKECEELEEKNIK EFLQSFVHIVQMFINTSGSGATNFSLLKQAGDVEENPGPMALLLALSLLVLWTS PAPTLSGTNDAEDCCLSVTQKPIPGYIVRNFHYLLIKDGCRVPAVVFTTLRGRQL CAPPDQPWVERIIQRLQRTSAKMKRRSS (SEQ ID NO: 15) (also referred to herein as ssV_{H}-P4-CAR-P2A-sushi15-P2A-CCL19 or P4-BB-sushi15-CCL19). In some cases, the isolated nucleic acid molecule encodes a polypeptide comprising about 85% sequence identity to SEQ ID NO: 15. In some cases, the isolated nucleic acid molecule encodes a polypeptide comprising about 90% sequence identity to SEQ ID NO: 15. In some cases, the isolated nucleic acid molecule encodes a polypeptide comprising about 95% sequence identity to SEQ ID NO: 15. In some cases, the isolated nucleic acid molecule encodes a polypeptide comprising about 96% sequence identity to SEQ ID NO: 15. In some cases, the isolated nucleic acid molecule encodes a polypeptide comprising about 97% sequence identity to SEQ ID NO: 15. In some cases, the isolated nucleic acid molecule encodes a polypeptide comprising about 98% sequence identity to SEQ ID NO: 15. In some cases, the isolated nucleic acid molecule encodes a polypeptide comprising about 99% sequence identity to SEQ ID NO: 15. In some cases, the isolated nucleic acid molecule encodes a polypeptide comprising SEQ ID NO: 15. In some cases, the isolated nucleic acid molecule encodes a polypeptide consisting of SEQ ID NO: 15.

In some embodiments, the isolated nucleic acid molecule encoding ssV_{H}-P4-CAR-P2A-sushi15-P2A-CCL19 or P4-BB-sushi15-CCL19 comprises a sequence comprising about 80%, 85%, 90%, 95%, 96%, 97%, 98%, 99%, or 100% sequence identity to ATGGATTGGACCTGGCGAATACTCTTCCTCGTCGCAGCGGCCACTGGTGCCC ATTCACAAGTCCAACTGCAGCAGAGCGGACCTGGCCTGGTGACACCCAGTC AGACTCTCAGCCTGACTTGTGCAATCAGCGGCGATAGTGTGTCTAGTAATTC TGCAACATGGAACTGGATCAGACAATCACCAAGTCGGGGACTGGAGTGGCT CGGTAGAACCTATTATAGGTCCAAATGGTATAACGATTATGCAGTGTCCGTG AAGTCCCGAATGTCTATCAACCCTGATACTAGTAAGAATCAATTCAGTCTGC AGCTTAACAGCGTAACCCCCGAAGATACTGCTGTGTATTACTGTGCCCGGG GTATGATGACTTACTACTACGGAATGGATGTGTGGGGGCAGGGAACAACCG TTACTGTTTCATCCGGCATTCTCGGGAGCGGAGGCGGTGGAAGCGGTGGGG GAGGGTCCGGGGGAGGAGGATCTCAGCCTGTTCTTACTCAATCTTCTTCCCT CTCCGCCTCACCCGGGGCCTCCGCCTCACTGACCTGCACTCTGCGATCAGGC ATCAACGTTGGGCCTTATAGAATCTACTGGTACCAGCAAAAGCCTGGATCA CCGCCCCAGTACCTGCTGAACTATAAATCAGACTCAGACAAGCAGCAGGGC TCCGGCGTGCCGAGTCGATTTAGCGGGAGCAAGGACGCGTCTGCTAATGCC GGCGTGCTTCTCATCAGCGGGCTCCGCAGTGAGGATGAGGCAGATTACTAC TGCATGATTTGGCATAGCAGTGCAGCCGTATTTGGCGGAGGAACACAGCTG ACTGTCCTCTCTCGCGCCGCCGCTCCGACCACCACCCCTGCACCACGCCCAC CTACTCCTGCGCCAACCATTGCCAGCCAGCCTCTCTCTCTCCGACCCGAGGC CTGTAGACCTGCCGCTGGCGGTGCAGTTCATACTCGGGGTCTCGATTTCGCC TGCGACATCTACATCTGGGCACCACTGGCTGGCACTTGTGGCGTTTTGCTCC TGTCCCTGGTGATCACTCTCTACTGTAATAAGAGGGGGAGGAAGAAACTCC TGTATATTTTCAAACAACCCTTTATGCGCCCTGTCCAAACCACCCAGGAAGA AGATGGATGTAGTTGCAGATTCCCAGAAGAAGAGGAGGGTGGGTGTGAACT TAGGGTGAAGTTTAGTCGCAGTGCCGACGCTCCCGCTTACCAACAGGGTCA GAACCAACTCTACAATGAGCTGAATCTGGGGAGGCGCGAAGAATACGACGT TCTGGATAAAAGACGCGGCCGCGACCCCGAGATGGGCGGGAAACCGCGGA GAAAGAACCCACAGGAAGGATTGTACAATGAGCTCCAGAAAGATAAGATG GCAGAAGCCTACTCCGAGATCGGCATGAAGGGGGAGCGAAGGCGCGGGAA AGGACACGATGGGCTGTACCAGGGTCTTTCAACCGCGACAAAGGACACCTA TGATGCTCTCCATATGCAGGCCCTCCCGCCACGCGGAAGTGGAGCAACTAA TTTTAGCCTTCTGAAACAAGCTGGCGATGTTGAGGAAAATCCTGGGCCGAT GGCACCTAGACGGGCACGCGGGTGTAGAACGCTGGGCCTCCCCGCACTGTT GTTGCTCTTGCTTCTGAGACCTCCCGCTACAAGGGGGATAACTTGCCCTCCA CCTATGAGCGTCGAGCATGCTGACATTTGGGTGAAGTCCTATTCACTCTATT CCCGGGAGCGGTACATCTGTAACTCTGGATTCAAGAGGAAAGCCGGCACCA GCAGTCTGACCGAGTGCGTGCTGAATAAGGCCACCAATGTGGCCCACTGGA CAACCCCTAGCCTTAAATGTATACGGTCAGGGGGCGGATCTGGAGGCGGCG GCTCCGGTGGAGGCGGGAGTGGGGGCGGGGGCTCTGGAGGTGGTAGCCTGC AGAATTGGGTTAACGTGATTAGCGACCTCAAAAAAATCGAAGATCTTATCC AGAGCATGCATATAGACGCAACCCTGTACACAGAAAGCGATGTTCACCCGT CCTGCAAGGTAACGGCTATGAAGTGTTTTCTTTTGGAGTTGCAAGTCATATC ACTGGAAAGTGGGGATGCCTCAATTCACGATACCGTGGAGAACCTCATCAT CCTCGCAAATAACAGCCTGAGCTCCAATGGCAATGTCACAGAGTCAGGTTG CAAAGAGTGTGAAGAGCTGGAAGAGAAAAACATCAAAGAGTTCCTCCAGT CATTTGTGCACATTGTCCAGATGTTCATTAACACTAGTGGTAGTGGTGCCAC AAATTTTAGTCTGTTGAAACAGGCCGGGGACGTCGAAGAAAACCCGGGGCC TATGGCCCTCTTGCTCGCACTGTCCCTCCTGGTCCTGTGGACATCACCCGCC CCCACCCTGTCCGGCACGAATGACGCAGAAGACTGCTGCCTGTCTGTCACG CAGAAACCCATCCCCGGCTATATAGTGCGGAACTTCCATTACCTGCTGATCA AGGACGGATGTAGGGTGCCAGCCGTCGTCTTCACCACCCTGCGAGGGCGCC AGCTGTGCGCTCCTCCTGACCAGCCCTGGGTGGAGCGGATCATTCAACGCTT GCAGCGCACCTCAGCAAAAATGAAAAGAAGAAGTAGT (SEQ ID NO: 40). In some cases, the isolated nucleic acid molecule comprises a sequence comprising about 85% sequence identity to SEQ ID NO: 40. In some cases, the isolated nucleic acid molecule comprises a sequence comprising about 90% sequence identity to SEQ ID NO: 40. In some cases, the isolated nucleic acid molecule comprises a sequence comprising about 95% sequence identity to SEQ ID NO: 40. In some cases, the isolated nucleic acid molecule comprises a sequence comprising about 96% sequence identity to SEQ ID NO: 40. In some cases, the isolated nucleic acid molecule comprises a sequence comprising about 97% sequence identity to SEQ ID NO: 40. In some cases, the isolated nucleic acid molecule comprises a sequence comprising about 98% sequence identity to SEQ ID NO: 40. In some cases, the isolated nucleic acid molecule comprises a sequence comprising about 99% sequence identity to SEQ ID NO: 40. In some cases, the isolated nucleic acid molecule comprises SEQ ID NO: 40. In some cases, the isolated nucleic acid molecule consists of SEQ ID NO: 40.

In some embodiments, the isolated nucleic acid molecule comprises a polynucleotide encoding a CAR that specifically binds to mesothelin and a polynucleotide encoding mIL15/Ra-LSP. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSRAAAPTTTPAPRPPTPAPTIASQPLSLRPEACRPAAGGAVHT RGLDFACDIYIWAPLAGTCGVLLLSLVITLYCNKRGRKKLLYIFKQPFMRPVQ TTQEEDGCSCRFPEEEEGGCELRVKFSRSADAPAYQQGQNQLYNELNLGRREE YDVLDKRRGRDPEMGGKPRRKNPQEGLYNELQKDKMAEAYSEIGMKGERRR GKGHDGLYQGLSTATKDTYDALHMQALPPRGSGATNFSLLKQAGDVEENPGP MRISKPHLRSISIQCYLCLLLNSHFLTEAGIHVFILGCFSAGLPKTEANWVNVISD LKKIEDLIQSMHIDATLYTESDVHPSCKVTAMKCFLLELQVISLESGDASIHDTV ENLIILANNSLSSNGNVTESGCKECEELEEKNIKEFLQSFVHIVQMFINTSSGGGS GGGGSGGGGSGGGSLQITCPPPMSVEHADIWVKSYSLYSRERYICNSGF KRKAGTSSLTECVLNKATNVAHWTTPSLKCIRDPAL VHQRPAPPSTVTTAGVTP QPESLSPSGKEPAASSPSSNNTAATTAAIVPGSQLMPSKSPSTGTTEISSHESSHG TPSQTTAKNWELTASASHQPPGVYPQGHSDTTVAISTSTVLLCGLSAVSLLACY LKSRQTPPLASVEMEAMEALPVTWGTSSRDEDLENCSHHL (SEQ ID NO: 53) (also referred to herein as ssVHsp-P4-CD8h/TM-BB-CD3z-P2A-IL15LSP-IL 15propeptide-mIL15/Ra or ssVHsp-P4-BB-mIL15/Ra-LSP). In some cases, the isolated nucleic acid molecule encodes a polypeptide comprising about 85% sequence identity to SEQ ID NO: 53. In some cases, the isolated nucleic acid molecule encodes a polypeptide comprising about 90% sequence identity to SEQ ID NO: 53. In some cases, the isolated nucleic acid molecule encodes a polypeptide comprising about 95% sequence identity to SEQ ID NO: 53. In some cases, the isolated nucleic acid molecule encodes a polypeptide comprising about 96% sequence identity to SEQ ID NO: 53. In some cases, the isolated nucleic acid molecule encodes a polypeptide comprising about 97% sequence identity to SEQ ID NO: 53. In some cases, the isolated nucleic acid molecule encodes a polypeptide comprising about 98% sequence identity to SEQ ID NO: 53. In some cases, the isolated nucleic acid molecule encodes a polypeptide comprising about 99% sequence identity to SEQ ID NO: 53. In some cases, the isolated nucleic acid molecule encodes a polypeptide comprising SEQ ID NO: 53. In some cases, the isolated nucleic acid molecule encodes a polypeptide consisting of SEQ ID NO: 53.

In some embodiments, the isolated nucleic acid molecule encoding ssVHsp-P4-CD8h/TM-BB-CD3z-P2A-IL15LSP-IL15propeptide-mIL15/Ra or ssVHsp-P4-BB-mIL15/Ra-LSP comprises a sequence comprising about 80%, 85%, 90%, 95%, 96%, 97%, 98%, 99%, or 100% sequence identity to ATGGACTGGACATGGCGGATACTCTTCCTCGTCGCTGCTGCAACCGGAGCCC ACAGCCAGGTGCAGCTCCAGCAGTCTGGGCCAGGTTTGGTGACTCCTAGTC AGACATTGAG CTTGACTTGTGCTATCAGCGGAGACTCTGTTTCATCTAATTCTGCAACTTGG AACTGGATTCGGCAGTCCCCCAGCCGGGGGCTCGAGTGGTTGGGTCGGACC TACTATCGGAGCAAATGGTACAATGACTATGCAGTGAGCGTCAAATCAAGA ATGAGCATCAATCCTGACACAAGCAAGAACCAGTTTAGCCTTCAGCTTAAT AGCGTGACTCCAGAGGACACAGCTGTGTACTATTGCGCGAGAGGCATGATG ACATACTATTACGGAATGGACGTGTGGGGCCAGGGAACTACTGTTACAGTG TCAAGCGGAATCCTCGGTAGCGGAGGCGGCGGTTCCGGCGGAGGGGGTAGT GGTGGCGGGGGTAGTCAACCTGTGCTGACCCAGAGCAGCTCTCTTAGTGCT AGCCCAGGTGCAAGTGCAAGTCTTACCTGTACACTGCGCTCCGGTATTAATG TGGGCCCTTACCGAATTTACTGGTACCAGCAGAAACCAGGCTCCCCTCCCCA GTATCTGCTGAACTATAAGTCTGACTCAGACAAACAGCAGGGCTCCGGTGT GCCATCCCGATTTAGTGGCTCAAAGGATGCTAGTGCAAATGCCGGTGTTCTC CTGATCAGCGGACTCAGATCAGAGGACGAAGCAGACTATTACTGTATGATT TGGCATAGCAGCGCTGCTGTCTTCGGAGGAGGGACTCAGCTCACTGTCTTGA GTCGGGCCGCTGCACCTACCACTACCCCTGCCCCTCGACCCCCTACTCCCGC CCCAACTATCGCATCCCAACCACTCTCTCTCAGACCCGAAGCCTGTAGACCC GCAGCCGGTGGCGCTGTGCATACTCGCGGACTTGATTTTGCTTGTGATATTT ATATCTGGGCCCCCCTTGCCGGAACTTGTGGAGTTCTCCTGCTGTCTCTCGT AATCACCCTTTATTGTAACAAACGGGGGCGCAAAAAACTTCTTTACATTTTC AAGCAGCCCTTTATGCGGCCCGTGCAGACCACACAGGAAGAAGATGGCTGC AGCTGCAGGTTCCCAGAGGAAGAAGAGGGCGGCTGCGAGCTGCGAGTAAA GTTCAGCCGGAGCGCCGATGCACCTGCATACCAGCAGGGTCAGAACCAGCT CTACAATGAGCTGAACCTGGGCAGAAGAGAGGAATATGATGTACTCGACAA GAGAAGGGGACGCGATCCAGAGATGGGCGGCAAACCACGGCGGAAAAATC CGCAGGAGGGGCTCTATAACGAGCTCCAGAAGGACAAGATGGCAGAAGCC TACTCAGAAATTGGCATGAAAGGAGAGAGAAGGAGGGGAAAGGGCCATGA TGGCCTTTACCAAGGGTTGTCTACTGCCACCAAGGATACGTACGATGCACTC CATATGCAGGCTCTTCCTCCCCGAGGCTCAGGAGCCACCAACTTCTCCCTGC TGAAGCAGGCCGGCGACGTGGAGGAGAACCCAGGTCCTATGAGAATCTCAA AACCCCATCTTAGAAGCATCTCTATACAGTGTTATCTGTGTCTCTTGCTGAA CTCCCACTTTTTGACAGAAGCTGGGATACATGTCTTTATCCTGGGATGTTTTT CCGCCGGGCTCCCTAAAACCGAGGCCAACTGGGTAAACGTAATCTCAGACC TTAAAAAGATTGAGGACCTGATTCAGTCAATGCATATCGATGCAACTTTGTA CACGGAGAGCGATGTTCACCCAAGTTGTAAAGTGACCGCGATGAAATGTTT TCTCCTCGAATTGCAGGTGATCTCCCTCGAGTCAGGCGACGCGTCTATCCAC GATACTGTGGAAAACCTTATCATTTTGGCGAACAATAGCCTCTCATCTAATG GTAACGTGACCGAGTCCGGCTGCAAGGAATGTGAGGAACTGGAGGAGAAA AATATCAAGGAATTCCTGCAGTCATTTGTACACATCGTGCAAATGTTTATCA ACACTTCTTCAGGAGGCGGGTCAGGAGGGGGAGGCTCAGGCGGCGGAGGT AGTGGAGGAGGAGGAAGTGGAGGCGGCAGTCTCCAGATCACCTGTCCACCA CCAATGAGTGTGGAACACGCGGACATTTGGGTCAAGTCATATTCTCTTTACT CCAGAGAGCGATACATATGCAACAGTGGTTTCAAGCGGAAAGCGGGTACTT CTTCACTTACCGAGTGCGTGCTCAATAAAGCAACCAACGTCGCGCACTGGA CAACACCTAGCCTGAAATGCATAAGAGATCCTGCCCTGGTTCACCAGCGGC CAGCGCCACCGTCCACAGTGACAACAGCTGGTGTGACACCCCAGCCGGAGA GCCTTAGCCCTAGCGGCAAAGAGCCGGCCGCAAGCTCACCAAGCTCAAATA ACACAGCCGCGACAACTGCTGCTATCGTGCCCGGTTCACAATTGATGCCGA GCAAATCACCAAGCACCGGAACTACCGAAATCTCAAGTCATGAAAGTAGTC ACGGTACTCCTAGCCAGACGACGGCAAAGAATTGGGAGCTGACTGCCTCTG CGAGCCACCAGCCGCCGGGTGTTTACCCTCAGGGGCATTCAGATACTACTGT GGCTATCTCTACTTCCACCGTCCTCTTGTGCGGCTTGTCTGCTGTGTCTCTTC TGGCTTGCTATTTGAAAAGTAGACAGACACCACCCCTTGCAAGTGTCGAGA TGGAAGCGATGGAGGCATTGCCTGTGACCTGGGGAACCAGTAGTAGGGACG AGGACCTGGA AAATTGTAGTCACCACCTGTGA (SEQ ID NO: 58). In some cases, the isolated nucleic acid molecule comprises a sequence comprising about 85% sequence identity to SEQ ID NO: 58. In some cases, the isolated nucleic acid molecule comprises a sequence comprising about 90% sequence identity to SEQ ID NO: 58. In some cases, the isolated nucleic acid molecule comprises a sequence comprising about 95% sequence identity to SEQ ID NO: 58. In some cases, the isolated nucleic acid molecule comprises a sequence comprising about 96% sequence identity to SEQ ID NO: 58. In some cases, the isolated nucleic acid molecule comprises a sequence comprising about 97% sequence identity to SEQ ID NO: 58. In some cases, the isolated nucleic acid molecule comprises a sequence comprising about 98% sequence identity to SEQ ID NO: 58. In some cases, the isolated nucleic acid molecule comprises a sequence comprising about 99% sequence identity to SEQ ID NO: 58. In some cases, the isolated nucleic acid molecule comprises SEQ ID NO: 58. In some cases, the isolated nucleic acid molecule consists of SEQ ID NO: 58.

In some embodiments, the isolated nucleic acid molecule comprises a polynucleotide encoding a CAR that specifically binds to mesothelin and a polynucleotide encoding sIL15-LSP. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSSGILGSGGGGSGGGGS GGGGSQPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSRAAAPTTTPAPRPPTPAPTIASQPLSLRPEACRPAAGGAVHT RGLDFACDIYIWAPLAGTCGVLLLSLVITLYCNKRGRKKLLYIFKQPFMRPVQT TQEEDGCSCRFPEEEEGGCELRVKFSRSADAPAYQQGQNQLYNELNLGRREEY DVLDKRRGRDPEMGGKPRRKNPQEGLYNELQKDKMAEAYSEIGMKGERRRG KGHDGLYQGLSTATKDTYDALHMQALPPRGSGATNFSLLKQAGDVEENPGPM RISKPHLRSISIQCYLCLLLNSHFLTEAGIHVFILGCFSAGLPKTEANWVNVISDL KKIEDLIQSMHIDATLYTESDVHPSCKVTAMKCFLLELQVISLESGDASIHDTVE NLIILANNSLSSNGNVTESGCKECEELEEKNIKEFLQSFVHIVQMFINTS (SEQ ID NO: 54) (also referred to herein as ssVHsp-P4-CD8h/TM-BB-CD3z-P2A-IL15LSP-IL 15propeptide-IL15 or ssVHsp-P4-BB-sIL15-LSP). In some cases, the isolated nucleic acid molecule encodes a polypeptide comprising about 85% sequence identity to SEQ ID NO: 54. In some cases, the isolated nucleic acid molecule encodes a polypeptide comprising about 90% sequence identity to SEQ ID NO: 54. In some cases, the isolated nucleic acid molecule encodes a polypeptide comprising about 95% sequence identity to SEQ ID NO: 54. In some cases, the isolated nucleic acid molecule encodes a polypeptide comprising about 96% sequence identity to SEQ ID NO: 54. In some cases, the isolated nucleic acid molecule encodes a polypeptide comprising about 97% sequence identity to SEQ ID NO: 54. In some cases, the isolated nucleic acid molecule encodes a polypeptide comprising about 98% sequence identity to SEQ ID NO: 54. In some cases, the isolated nucleic acid molecule encodes a polypeptide comprising about 99% sequence identity to SEQ ID NO: 54. In some cases, the isolated nucleic acid molecule encodes a polypeptide comprising SEQ ID NO: 54. In some cases, the isolated nucleic acid molecule encodes a polypeptide consisting of SEQ ID NO: 54.

In some embodiments, the isolated nucleic acid molecule encoding ssVHsp-P4-CD8h/TM-BB-CD3z-P2A-IL15LSP-IL15propeptide-IL15 or ssVHsp-P4-BB-sIL 15-LSP comprises a sequence comprising about 80%, 85%, 90%, 95%, 96%, 97%, 98%, 99%, or 100% sequence identity to ATGGACTGGACATGGCGGATACTCTTCCTCGTCGCTGCTGCAACCGGAGCCC ACAGCCAGGTGCAGCTCCAGCAGTCTGGGCCAGGTTTGGTGACTCCTAGTC AGACATTGAGCTTGACTTGTGCTATCAGCGGAGACTCTGTTTCATCTAATTC TGCAACTTGGAACTGGATTCGGCAGTCCCCCAGCCGGGGGCTCGAGTGGTT GGGTCGGACCTACTATCGGAGCAAATGGTACAATGACTATGCAGTGAGCGT CAAATCAAGAATGAGCATCAATCCTGACACAAGCAAGAACCAGTTTAGCCT TCAGCTTAATAGCGTGACTCCAGAGGACACAGCTGTGTACTATTGCGCGAG AGGCATGATGACATACTATTACGGAATGGACGTGTGGGGCCAGGGAACTAC TGTTACAGTGTCAAGCGGAATCCTCGGTAGCGGAGGCGGCGGTTCCGGCGG AGGGGGTAGTGGTGGCGGGGGTAGTCAACCTGTGCTGACCCAGAGCAGCTC TCTTAGTGCTAGCCCAGGTGCAAGTGCAAGTCTTACCTGTACACTGCGCTCC GGTATTAATGTGGGCCCTTACCGAATTTACTGGTACCAGCAGAAACCAGGC TCCCCTCCCCAGTATCTGCTGAACTATAAGTCTGACTCAGACAAACAGCAGG GCTCCGGTGTGCCATCCCGATTTAGTGGCTCAAAGGATGCTAGTGCAAATGC CGGTGTTCTCCTGATCAGCGGACTCAGATCAGAGGACGAAGCAGACTATTA CTGTATGATTTGGCATAGCAGCGCTGCTGTCTTCGGAGGAGGGACTCAGCTC ACTGTCTTGAGTCGGGCCGCTGCACCTACCACTACCCCTGCCCCTCGACCCC CTACTCCCGCCCCAACTATCGCATCCCAACCACTCTCTCTCAGACCCGAAGC CTGTAGACCCGCAGCCGGTGGCGCTGTGCATACTCGCGGACTTGATTTTGCT TGTGATATTTATATCTGGGCCCCCCTTGCCGGAACTTGTGGAGTTCTCCTGCT GTCTCTCGTAATCACCCTTTATTGTAACAAACGGGGGCGCAAAAAACTTCTT TACATTTTCAAGCAGCCCTTTATGCGGCCCGTGCAGACCACACAGGAAGAA GATGGCTGCAGCTGCAGGTTCCCAGAGGAAGAAGAGGGCGGCTGCGAGCT GCGAGTAAAGTTCAGCCGGAGCGCCGATGCACCTGCATACCAGCAGGGTCA GAACCAGCTCTACAATGAGCTGAACCTGGGCAGAAGAGAGGAATATGATGT ACTCGACAAGAGAAGGGGACGCGATCCAGAGATGGGCGGCAAACCACGGC GGAAAAATCCGCAGGAGGGGCTCTATAACGAGCTCCAGAAGGACAAGATG GCAGAAGCCTACTCAGAAATTGGCATGAAAGGAGAGAGAAGGAGGGGAAA GGGCCATGATGGCCTTTACCAAGGGTTGTCTACTGCCACCAAGGATACGTA CGATGCACTCCATATGCAGGCTCTTCCTCCCCGAGGTTCAGGCGCAACAAAT TTTTCACTTCTTAAACAAGCTGGCGATGTCGAGGAAAACCCAGGTCCCATGC GGATCTCTAAACCCCACTTGCGGAGCATTTCTATCCAGTGTTATCTTTGCCTC CTGCTTAACTCCCACTTTCTCACAGAAGCAGGGATACACGTGTTCATCCTGG GCTGTTTTTCTGCCGGTCTCCCCAAAACAGAAGCCAACTGGGTGAATGTGAT CAGTGATCTTAAGAAAATAGAAGACCTCATCCAGTCAATGCACATCGATGC CACCTTGTACACTGAGAGCGACGTGCACCCTTCCTGCAAGGTGACAGCTAT GAAGTGCTTCCTGCTTGAGCTCCAGGTCATATCCCTTGAGTCTGGAGATGCA AGTATCCACGATACGGTGGAAAACCTTATTATACTGGCCAATAATTCTCTTT CTTCCAATGGCAATGTTACCGAATCAGGGTGTAAAGAGTGCGAAGAGCTGG AGGAGAAAAATATCAAAGAGTTTTTGCAGTCATTTGTGCACATCGTCCAGA TGTTTATTAATACAAGTTGA (SEQ ID NO: 59). In some cases, the isolated nucleic acid molecule comprises a sequence comprising about 85% sequence identity to SEQ ID NO: 59. In some cases, the isolated nucleic acid molecule comprises a sequence comprising about 90% sequence identity to SEQ ID NO: 59. In some cases, the isolated nucleic acid molecule comprises a sequence comprising about 95% sequence identity to SEQ ID NO: 59. In some cases, the isolated nucleic acid molecule comprises a sequence comprising about 96% sequence identity to SEQ ID NO: 59. In some cases, the isolated nucleic acid molecule comprises a sequence comprising about 97% sequence identity to SEQ ID NO: 59. In some cases, the isolated nucleic acid molecule comprises a sequence comprising about 98% sequence identity to SEQ ID NO: 59. In some cases, the isolated nucleic acid molecule comprises a sequence comprising about 99% sequence identity to SEQ ID NO: 59. In some cases, the isolated nucleic acid molecule comprises SEQ ID NO: 59. In some cases, the isolated nucleic acid molecule consists of SEQ ID NO: 59.

In some embodiments, the isolated nucleic acid molecule comprises a polynucleotide encoding a CAR that specifically binds to mesothelin, a polynucleotide encoding mIL15/Ra-LSP and a polynucleotide encoding CCL19. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSSGILGSGGGGSGGGGS GGGGSQPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLSRAAAPTTTPAPRPPTPAPTIASQPLSLRPEACRPAAGGAVHT RGLDFACDIYIWAPLAGTCGVLLLSLVITLYCNKRGRKKLL YIFKQPFMRPVQT TQEEDGCSCRFPEEEEGGCELRVKFSRSADAPAYQQGQNQLYNELNLGRREEY DVLDKRRGRDPEMGGKPRRKNPQEGLYNELQKDKMAEAYSEIGMKGERRRG KGHDGLYQGLSTATKDTYDALHMQALPPRGSGATNFSLLKQAGDVEENPGPM RISKPHLRSISIQCYLCLLLNSHFLTEAGIHVFILGCFSAGLPKTEANWVNVISDL KKIEDLIQSMHIDATLYTESDVHPSCKVTAMKCFLLELQVISLESGDASIHDTVE NLIILANNSLSSNGNVTESGCKECEELEEKNIKEFLQSFVHIVQMFINTSGSGATN FSLLKQAGDVEENPGPMALLLALSLLVLWTSPAPTLSGTNDAEDCCLSVTQKPI PGYIVRNFHYLLIKDGCRVPAVVFTTLRGRQLCAPPDQPWVERIIQRLQRTSAK MKRRSS (SEQ ID NO: 55) (also referred to herein as ssVHsp-P4-CD8h/TM-BB-CD3z-P2A-IL15LSP-IL15propeptide-IL15-P2A-endospCCL19 or ssVHsp-P4-BB-mIL15/Ra-LSP-CCL19). In some cases, the isolated nucleic acid molecule encodes a polypeptide comprising about 85% sequence identity to SEQ ID NO: 55. In some cases, the isolated nucleic acid molecule encodes a polypeptide comprising about 90% sequence identity to SEQ ID NO: 55. In some cases, the isolated nucleic acid molecule encodes a polypeptide comprising about 95% sequence identity to SEQ ID NO: 55. In some cases, the isolated nucleic acid molecule encodes a polypeptide comprising about 96% sequence identity to SEQ ID NO: 55. In some cases, the isolated nucleic acid molecule encodes a polypeptide comprising about 97% sequence identity to SEQ ID NO: 55. In some cases, the isolated nucleic acid molecule encodes a polypeptide comprising about 98% sequence identity to SEQ ID NO: 55. In some cases, the isolated nucleic acid molecule encodes a polypeptide comprising about 99% sequence identity to SEQ ID NO: 55. In some cases, the isolated nucleic acid molecule encodes a polypeptide comprising SEQ ID NO: 55. In some cases, the isolated nucleic acid molecule encodes a polypeptide consisting of SEQ ID NO: 55.

In some embodiments, the isolated nucleic acid molecule encoding ssVHsp-P4-CD8h/TM-BB-CD3z-P2A-IL15LSP-IL15propeptide-IL15-P2A-endospCCL19 or ssVHsp-P4-BB-mIL15/Ra-LSP-CCL19 comprises a sequence comprising about 80%, 85%, 90%, 95%, 96%, 97%, 98%, 99%, or 100% sequence identity to ATGGATTGGACCTGGCGAATACTCTTCCTCGTCGCAGCGGCCACTGGTGCCC ATTCACAAGTCCAACTGCAGCAGAGCGGACCTGGCCTGGTGACACCCAGTC AGACTCTCAGCCTGACTTGTGCAATCAGCGGCGATAGTGTGTCTAGTAATTC TGCAACATGGAACTGGATCAGACAATCACCAAGTCGGGGACTGGAGTGGCT CGGTAGAACCTATTATAGGTCCAAATGGTATAACGATTATGCAGTGTCCGTG AAGTCCCGAATGTCTATCAACCCTGATACTAGTAAGAATCAATTCAGTCTGC AGCTTAACAGCGTAACCCCCGAAGATACTGCTGTGTATTACTGTGCCCGGG GTATGATGACTTACTACTACGGAATGGATGTGTGGGGGCAGGGAACAACCG TTACTGTTTCATCCGGCATTCTCGGGAGCGGAGGCGGTGGAAGCGGTGGGG GAGGGTCCGGGGGAGGAGGATCTCAGCCTGTTCTTACTCAATCTTCTTCCCT CTCCGCCTCACCCGGGGCCTCCGCCTCACTGACCTGCACTCTGCGATCAGGC ATCAACGTTGGGCCTTATAGAATCTACTGGTACCAGCAAAAGCCTGGATCA CCGCCCCAGTACCTGCTGAACTATAAATCAGACTCAGACAAGCAGCAGGGC TCCGGCGTGCCGAGTCGATTTAGCGGGAGCAAGGACGCGTCTGCTAATGCC GGCGTGCTTCTCATCAGCGGGCTCCGCAGTGAGGATGAGGCAGATTACTAC TGCATGATTTGGCATAGCAGTGCAGCCGTATTTGGCGGAGGAACACAGCTG ACTGTCCTCTCTCGCGCCGCCGCTCCGACCACCACCCCTGCACCACGCCCAC CTACTCCTGCGCCAACCATTGCCAGCCAGCCTCTCTCTCTCCGACCCGAGGC CTGTAGACCTGCCGCTGGCGGTGCAGTTCATACTCGGGGTCTCGATTTCGCC TGCGACATCTACATCTGGGCACCACTGGCTGGCACTTGTGGCGTTTTGCTCC TGTCCCTGGTGATCACTCTCTACTGTAATAAGAGGGGGAGGAAGAAACTCC TGTATATTTTCAAACAACCCTTTATGCGCCCTGTCCAAACCACCCAGGAAGA AGATGGATGTAGTTGCAGATTCCCAGAAGAAGAGGAGGGTGGGTGTGAACT TAGGGTGAAGTTTAGTCGCAGTGCCGACGCTCCCGCTTACCAACAGGGTCA GAACCAACTCTACAATGAGCTGAATCTGGGGAGGCGCGAAGAATACGACGT TCTGGATAAAAGACGCGGCCGCGACCCCGAGATGGGCGGGAAACCGCGGA GAAAGAACCCACAGGAAGGATTGTACAATGAGCTCCAGAAAGATAAGATG GCAGAAGCCTACTCCGAGATCGGCATGAAGGGGGAGCGAAGGCGCGGGAA AGGACACGATGGGCTGTACCAGGGTCTTTCAACCGCGACAAAGGACACCTA TGATGCTCTCCATATGCAGGCCCTCCCGCCACGCGGAAGTGGAGCAACTAA TTTTAGCCTTCTGAAACAAGCTGGCGATGTTGAGGAAAATCCTGGGCCGAT GCGCATTAGCAAGCCACATCTGAGGAGTATCAGCATCCAGTGCTACCTTTGC CTGCTGCTCAACTCTCACTTTCTGACAGAAGCTGGCATCCACGTCTTCATCC TGGGGTGCTTCAGCGCCGGCTTGCCGAAGACCGAAGCCAACTGGGTGAATG TGATCTCCGACCTCAAGAAGATCGAGGACCTGATCCAGAGTATGCATATTG ATGCTACACTTTACACCGAGTCCGATGTTCACCCTAGTTGTAAGGTGACTGC CATGAAATGTTTCTTGCTGGAGCTTCAGGTAATAAGCCTTGAGTCTGGGGAT GCAAGCATTCATGACACGGTTGAGAATCTCATCATCCTGGCAAATAATTCAC TGTCTTCAAATGGTAACGTTACAGAGAGCGGCTGTAAGGAGTGCGAAGAGC TTGAAGAGAAAAACATCAAGGAATTCCTCCAGAGTTTCGTGCACATCGTGC AAATGTTCATCAACACGAGCTCTGGAGGCGGATCAGGAGGCGGAGGATCAG GGGGGGGAGGGTCAGGCGGAGGGGGATCTGGTGGAGGCAGCCTTCAAATC ACATGCCCGCCACCTATGTCCGTTGAGCACGCCGACATATGGGTGAAGTCA TATTCACTGTATAGTCGGGAGAGGTACATTTGTAATTCAGGTTTCAAGCGAA AAGCTGGGACATCAAGCCTGACAGAATGCGTACTTAACAAGGCCACAAATG TCGCCCATTGGACCACTCCGAGTCTGAAGTGTATACGAGATCCCGCACTGGT GCACCAGCGACCTGCTCCCCCTAGTACAGTAACAACCGCGGGCGTTACGCC CCAGCCTGAATCCCTGAGCCCATCTGGCAAGGAGCCTGCAGCTAGCTCTCC GAGCAGCAATAATACTGCAGCGACCACTGCAGCCATCGTCCCCGGCTCCCA GCTCATGCCTAGTAAAAGTCCGTCTACAGGAACGACCGAAATCTCCAGCCA CGAGTCTAGTCACGGGACCCCGAGTCAGACCACTGCCAAGAACTGGGAGCT TACGGCCAGTGCCTCCCATCAACCCCCGGGCGTCTACCCGCAAGGCCATAG CGACACCACAGTCGCCATTAGCACATCTACTGTCCTCTTGTGCGGGCTCTCC GCTGTGTCCCTCCTGGCCTGTTATCTCAAGAGCAGACAGACCCCCCCATTGG CAAGCGTTGAGATGGAGGCAATGGAGGCTCTGCCCGTTACTTGGGGGACTT CTTCACGCGACGAGGATCTGGAGAACTGCTCCCACCACCTGGGAAGTGGTG CCACAAATTTCAGCCTGCTCAAGCAGGCCGGGGATGTTGAAGAGAACCCAG GGCCGATGGCCCTCTTGCTCGCACTGTCCCTCCTGGTCCTGTGGACATCACC CGCCCCCACCCTGTCCGGCACGAATGACGCAGAAGACTGCTGCCTGTCTGTC ACGCAGAAACCCATCCCCGGCTATATAGTGCGGAACTTCCATTACCTGCTGA TCAAGGACGGATGTAGGGTGCCAGCCGTCGTCTTCACCACCCTGCGAGGGC GCCAGCTGTGCGCTCCTCCTGACCAGCCCTGGGTGGAGCGGATCATTCAAC GCTTGCAGCGCACCTCAGCAAAAATGAAAAGAAGAAGTAGTTGA (SEQ ID NO: 60). In some cases, the isolated nucleic acid molecule comprises a sequence comprising about 85% sequence identity to SEQ ID NO: 60. In some cases, the isolated nucleic acid molecule comprises a sequence comprising about 90% sequence identity to SEQ ID NO: 60. In some cases, the isolated nucleic acid molecule comprises a sequence comprising about 95% sequence identity to SEQ ID NO: 60. In some cases, the isolated nucleic acid molecule comprises a sequence comprising about 96% sequence identity to SEQ ID NO: 60. In some cases, the isolated nucleic acid molecule comprises a sequence comprising about 97% sequence identity to SEQ ID NO: 60. In some cases, the isolated nucleic acid molecule comprises a sequence comprising about 98% sequence identity to SEQ ID NO: 60. In some cases, the isolated nucleic acid molecule comprises a sequence comprising about 99% sequence identity to SEQ ID NO: 60. In some cases, the isolated nucleic acid molecule comprises SEQ ID NO: 60. In some cases, the isolated nucleic acid molecule consists of SEQ ID NO: 60.

In some embodiments, the isolated nucleic acid molecule comprises a polynucleotide encoding a CAR that specifically binds to mesothelin, a polynucleotide encoding sIL15-LSP and a polynucleotide encoding CCL19. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLN SVTPEDTAVYYCARGMMTYYYGMDVWGQGTTVTVSS [symbol] [symbol] QPVLTQSSSLSASPGASASLTCTLRSGINVGPYRIYWYQQKPGSPPQYLL NYKSDSDKQQGSGVPSRFSGSKDASANAGVLLISGLRSEDEADYYCMIWHSSA AVFGGGTQLTVLS [symbol] PTTTPAPRPPTPAPTIASQPLSLRPEACRPAAGGAVHT RGLDFACDIYIWAPLAGTCGVLLLSLVITLYCNKRGRKKLLYIFKQPFMRPVQ TTQEEDGCSCRFPEEEEGGCELRVKFSRSADAPAYQQGQNQLYNELNLGRREE YDVLDKRRGRDPEMGGKPRRKNPQEGLYNELQKDKMAEAYSEIGMKGERRR GKGHDGLYQGLSTATKDTYDALHMQALPPRGSGATNFSLLKQAGDVEENPGP MRISKPHLRSISIQCYLCLLLNSHFLTEAGIHVFILGCFSAGLPKTEANWVNVISD LKKIEDLIQSMHIDATLYTESDVHPSCKVTAMKCFLLELQVISLESGDASIHDTV ENLIILANNSLSSNGNVTESGCKECEELEEKNIKEFLQSFVHIVQMFINTSGSGAT NFSLLKQAGDVEENPGPMALLLALSLLVLWTSPAPTLSGTNDAEDCCLSVTQK PIPGYIVRNFHYLLIKDGCRVPAVVFTTLRGRQLCAPPDQPWVERIIQRLQRTSA KMKRRSS (SEQ ID NO: 56) (also referred to herein as ssVHsp-P4-CD8h/TM-BB-CD3z-P2A-IL15LSP-IL15propeptide-IL15-P2A-endospCCL19 or ssVHsp-P4-BB-SIL15-LSP-CCL19). In some cases, the isolated nucleic acid molecule encodes a polypeptide comprising about 85% sequence identity to SEQ ID NO: 56. In some cases, the isolated nucleic acid molecule encodes a polypeptide comprising about 90% sequence identity to SEQ ID NO: 56. In some cases, the isolated nucleic acid molecule encodes a polypeptide comprising about 95% sequence identity to SEQ ID NO: 56. In some cases, the isolated nucleic acid molecule encodes a polypeptide comprising about 96% sequence identity to SEQ ID NO: 56. In some cases, the isolated nucleic acid molecule encodes a polypeptide comprising about 97% sequence identity to SEQ ID NO: 56. In some cases, the isolated nucleic acid molecule encodes a polypeptide comprising about 98% sequence identity to SEQ ID NO: 56. In some cases, the isolated nucleic acid molecule encodes a polypeptide comprising about 99% sequence identity to SEQ ID NO: 56. In some cases, the isolated nucleic acid molecule encodes a polypeptide comprising SEQ ID NO: 56. In some cases, the isolated nucleic acid molecule encodes a polypeptide consisting of SEQ ID NO: 56.

In some embodiments, the isolated nucleic acid molecule encoding ssVHsp-P4-CD8h/TM-BB-CD3z-P2A-IL15LSP-IL15propeptide-IL15-P2A-endospCCL19 or ssVHsp-P4-BB-sIL15-LSP-CCL19 comprises a sequence comprising about 80%, 85%, 90%, 95%, 96%, 97%, 98%, 99%, or 100% sequence identity to ATGGATTGGACCTGGCGAATACTCTTCCTCGTCGCAGCGGCCACTGGTGCCC ATTCACAAGTCCAACTGCAGCAGAGCGGACCTGGCCTGGTGACACCCAGTC AGACTCTCAGCCTGACTTGTGCAATCAGCGGCGATAGTGTGTCTAGTAATTC TGCAACATGGAACTGGATCAGACAATCACCAAGTCGGGGACTGGAGTGGCT CGGTAGAACCTATTATAGGTCCAAATGGTATAACGATTATGCAGTGTCCGTG AAGTCCCGAATGTCTATCAACCCTGATACTAGTAAGAATCAATTCAGTCTGC AGCTTAACAGCGTAACCCCCGAAGATACTGCTGTGTATTACTGTGCCCGGG GTATGATGACTTACTACTACGGAATGGATGTGTGGGGGCAGGGAACAACCG TTACTGTTTCATCCGGCATTCTCGGGAGCGGAGGCGGTGGAAGCGGTGGGG GAGGGTCCGGGGGAGGAGGATCTCAGCCTGTTCTTACTCAATCTTCTTCCCT CTCCGCCTCACCCGGGGCCTCCGCCTCACTGACCTGCACTCTGCGATCAGGC ATCAACGTTGGGCCTTATAGAATCTACTGGTACCAGCAAAAGCCTGGATCA CCGCCCCAGTACCTGCTGAACTATAAATCAGACTCAGACAAGCAGCAGGGC TCCGGCGTGCCGAGTCGATTTAGCGGGAGCAAGGACGCGTCTGCTAATGCC GGCGTGCTTCTCATCAGCGGGCTCCGCAGTGAGGATGAGGCAGATTACTAC TGCATGATTTGGCATAGCAGTGCAGCCGTATTTGGCGGAGGAACACAGCTG ACTGTCCTCTCTCGCGCCGCCGCTCCGACCACCACCCCTGCACCACGCCCAC CTACTCCTGCGCCAACCATTGCCAGCCAGCCTCTCTCTCTCCGACCCGAGGC CTGTAGACCTGCCGCTGGCGGTGCAGTTCATACTCGGGGTCTCGATTTCGCC TGCGACATCTACATCTGGGCACCACTGGCTGGCACTTGTGGCGTTTTGCTCC TGTCCCTGGTGATCACTCTCTACTGTAATAAGAGGGGGAGGAAGAAACTCC TGTATATTTTCAAACAACCCTTTATGCGCCCTGTCCAAACCACCCAGGAAGA AGATGGATGTAGTTGCAGATTCCCAGAAGAAGAGGAGGGTGGGTGTGAACT TAGGGTGAAGTTTAGTCGCAGTGCCGACGCTCCCGCTTACCAACAGGGTCA GAACCAACTCTACAATGAGCTGAATCTGGGGAGGCGCGAAGAATACGACGT TCTGGATAAAAGACGCGGCCGCGACCCCGAGATGGGCGGGAAACCGCGGA GAAAGAACCCACAGGAAGGATTGTACAATGAGCTCCAGAAAGATAAGATG GCAGAAGCCTACTCCGAGATCGGCATGAAGGGGGAGCGAAGGCGCGGGAA AGGACACGATGGGCTGTACCAGGGTCTTTCAACCGCGACAAAGGACACCTA TGATGCTCTCCATATGCAGGCCCTCCCGCCACGCGGAAGTGGAGCAACTAA TTTTAGCCTTCTGAAACAAGCTGGCGATGTTGAGGAAAATCCTGGGCCGAT GCGCATCTCCAAGCCCCATCTGAGGAGCATCAGCATCCAGTGCTACCTGTGT CTGCTGCTCAACAGCCACTTCCTGACGGAAGCAGGCATTCATGTCTTTATCC TGGGATGCTTTTCTGCCGGCCTGCCAAAGACAGAAGCAAACTGGGTTAACG TTATCAGTGATCTGAAAAAAATCGAGGACCTGATCCAGTCCATGCATATTG ACGCTACGCTGTATACAGAGTCCGACGTCCACCCATCATGCAAGGTGACCG CTATGAAGTGTTTCCTGCTGGAACTGCAGGTTATCAGCTTGGAAAGTGGCGA CGCTTCCATTCACGATACGGTGGAGAACTTGATAATCCTTGCGAATAATAGT CTGAGCAGCAACGGCAACGTTACTGAAAGCGGGTGCAAAGAATGTGAAGA GCTCGAAGAGAAAAACATCAAAGAATTTTTGCAGTCTTTCGTGCATATTGTT CAGATGTTTATTAACACCAGTGGATCAGGAGCAACTAACTTCTCTCTTCTTA AGCAAGCTGGCGATGTAGAGGAAAACCCTGGGCCTATGGCCCTCTTGCTCG CACTGTCCCTCCTGGTCCTGTGGACATCACCCGCCCCCACCCTGTCCGGCAC GAATGACGCAGAAGACTGCTGCCTGTCTGTCACGCAGAAACCCATCCCCGG CTATATAGTGCGGAACTTCCATTACCTGCTGATCAAGGACGGATGTAGGGT GCCAGCCGTCGTCTTCACCACCCTGCGAGGGCGCCAGCTGTGCGCTCCTCCT GACCAGCCCTGGGTGGAGCGGATCATTCAACGCTTGCAGCGCACCTCAGCA AAAATGAAAAGAAGAAGTAGTTGA (SEQ ID NO: 61). In some cases, the isolated nucleic acid molecule comprises a sequence comprising about 85% sequence identity to SEQ ID NO: 61. In some cases, the isolated nucleic acid molecule comprises a sequence comprising about 90% sequence identity to SEQ ID NO: 61. In some cases, the isolated nucleic acid molecule comprises a sequence comprising about 95% sequence identity to SEQ ID NO: 61. In some cases, the isolated nucleic acid molecule comprises a sequence comprising about 96% sequence identity to SEQ ID NO: 61. In some cases, the isolated nucleic acid molecule comprises a sequence comprising about 97% sequence identity to SEQ ID NO: 61. In some cases, the isolated nucleic acid molecule comprises a sequence comprising about 98% sequence identity to SEQ ID NO: 61. In some cases, the isolated nucleic acid molecule comprises a sequence comprising about 99% sequence identity to SEQ ID NO: 61. In some cases, the isolated nucleic acid molecule comprises SEQ ID NO: 61. In some cases, the isolated nucleic acid molecule consists of SEQ ID NO: 61.

In some embodiments, the isolated nucleic acid molecule comprises a polynucleotide encoding a CAR that specifically binds to mesothelin, a polynucleotide encoding sushi15 and a polynucleotide encoding CCL19. In some instances, the isolated nucleic acid molecule encodes a polypeptide comprising about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSAT WNWIRQSPSRGLEWLGRTYYRSKWYNDYAVSVKSRMSLNPDTSKNQFSLQLNS VTPEDTAVYYCARGMMTYYYGMDVWGQGTTVTVSS [symbol] [symbol] QPVLTQSSSLSASPGASASLTCTLRSGINVGPYRIYWYQQKPGSPPQYLLN YKSDSDKQQGSGVPSRFSGSKDASANAGVLLISGLRSEDEADYYCMIWHSSAA VFGGGTQLTVLS [symbol] IEVMYPPPYLDNEKSNGTIIHVKGKHLCPSPLFPGPS KPFWVLVVVGGVLACYSLLVTVAFIIFWVRSKRSRLLHSDYMNMTPRRPGP TRKHYQPYAPPRDFAAYRSRVKFSRSADAPAYQQGQNQLYNELNLGRREEYDV LDKRRGRDPEMGGKPRRKNPQEGLYNELQKDKMAEAYSEIGMKGERRRGKGH DGLYQGLSTATKDTYDALHMQALPPRGSGATNFSLLKQAGDVEENPGPMAPRR ARGCRTLGLPALLLLLLLRPPATRGITCPPPMSVEHADIWVKSYSLYSRERYICN SGFKRKAGTSSLTECVLNKATNVAHWTTPSLKCIRSGGSGGGGSGGGSGGGG SLQNWVNVISDLKKIEDLIQSMHIDATLYTESDVHPSCKVTAMKCFLLELQVIS LESGDASIHDTVENLIILANNSLSSNGNVTESGCKECEELEEKNIKEFLQSFVHIV QMFINTSGSGATNFSLLKQAGDVEENPGPMALLLALSLLVLWTSPAPTLSGTND AEDCCLSVTQKPIPGYIVRNFHYLLIKDGCRVPAVVFTTLRGRQLCAPPDQPWV ERIIQRLQRTSAKMKRRSS (SEQ ID NO: 57) (also referred to herein as ssV_{H}-P4-CD28hinge-TM-CD28cyto-CD3z-P2A-IL15Rasp-IL15Rα (sushi)-20aalinker-IL15-P2A-endospCCL19 or SSVHsp-P4-CD28-sushi15-CCL19). In some cases, the isolated nucleic acid molecule encodes a polypeptide comprising about 85% sequence identity to SEQ ID NO: 57. In some cases, the isolated nucleic acid molecule encodes a polypeptide comprising about 90% sequence identity to SEQ ID NO: 57. In some cases, the isolated nucleic acid molecule encodes a polypeptide comprising about 95% sequence identity to SEQ ID NO: 57. In some cases, the isolated nucleic acid molecule encodes a polypeptide comprising about 96% sequence identity to SEQ ID NO: 57. In some cases, the isolated nucleic acid molecule encodes a polypeptide comprising about 97% sequence identity to SEQ ID NO: 57. In some cases, the isolated nucleic acid molecule encodes a polypeptide comprising about 98% sequence identity to SEQ ID NO: 57. In some cases, the isolated nucleic acid molecule encodes a polypeptide comprising about 99% sequence identity to SEQ ID NO: 57. In some cases, the isolated nucleic acid molecule encodes a polypeptide comprising SEQ ID NO: 57. In some cases, the isolated nucleic acid molecule encodes a polypeptide consisting of SEQ ID NO: 57.

### Vectors

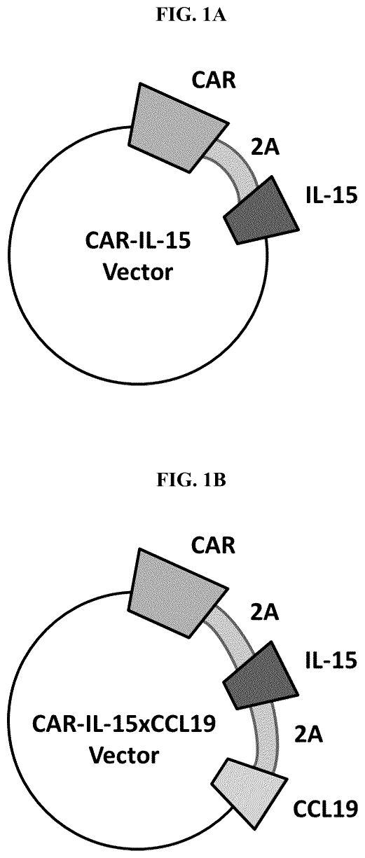

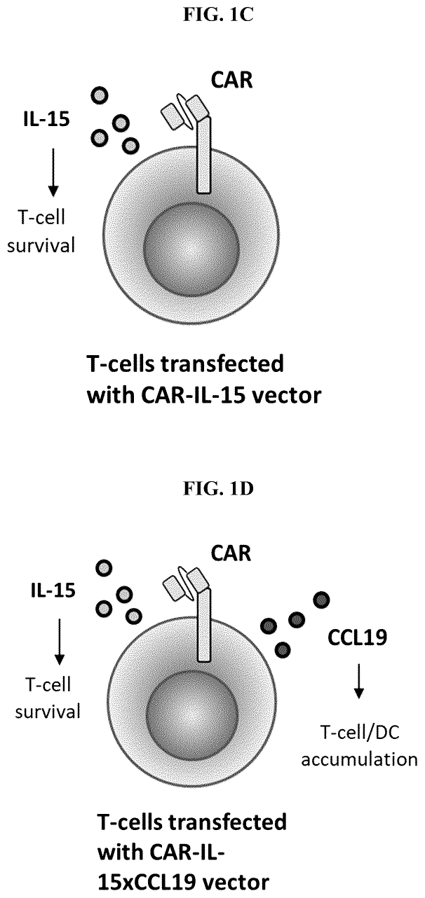

In some embodiments, one or more vectors encompass the polynucleotide encoding the CAR, the polynucleotide encoding IL-15, and optionally the polynucleotide encoding CCL19. In some embodiments, a vector (e.g., an expression vector) comprises the nucleic acid molecule comprising a polynucleotide encoding a chimeric antigen receptor (CAR) comprising an antibody that specifically recognizes human mesothelin, a CD8 hinge region, a CD8 transmembrane region, a 4-1BB intracellular region, and a CD3 (intracellular region; a polynucleotide encoding IL-15; and optionally a polynucleotide encoding CCL19. See FIG. 1A and FIG. 1B.

In some embodiments, the polynucleotide encoding the CAR and the polynucleotide encoding IL-15 are arranged in the vector (e.g., an expression vector) from the 5′ terminus to the 3′ terminus as:
the polynucleotide encoding the CAR—the polynucleotide encoding IL-15; or
the polynucleotide encoding IL-15—the polynucleotide encoding the CAR.

In some embodiments, the polynucleotide encoding the CAR, the polynucleotide encoding IL-15, and the polynucleotide encoding CCL19 are arranged in the vector (e.g., an expression vector) from the 5′ terminus to the 3′ terminus as:
the polynucleotide encoding the CAR—the polynucleotide encoding IL-15—the polynucleotide encoding CCL19;
the polynucleotide encoding the CAR—the polynucleotide encoding CCL19—the polynucleotide encoding IL-15;
the polynucleotide encoding IL-15—the polynucleotide encoding the CAR—the polynucleotide encoding CCL19;
the polynucleotide encoding CCL19—the polynucleotide encoding the CAR—the polynucleotide encoding IL-15;
the polynucleotide encoding IL-15—the polynucleotide encoding CCL19—the polynucleotide encoding the CAR; or
the polynucleotide encoding CCL19—the polynucleotide encoding IL-15—the polynucleotide encoding the CAR.

In some embodiments, a first vector (e.g., a first expression vector) comprises the polynucleotide encoding the CAR, and a second vector (e.g., a second expression vector) comprises the polynucleotide encoding IL-15.

In some embodiments, a first vector (e.g., a first expression vector) comprises the polynucleotide encoding the CAR, and a second vector (e.g., a second expression vector) comprises the polynucleotide encoding IL-15 and the polynucleotide encoding CCL19, in which the polynucleotide encoding IL-15 and the polynucleotide encoding CCL19 are optionally arranged in the second vector (e.g., the second expression vector) from the 5′ terminus to the 3′ terminus as the polynucleotide encoding IL-15—the polynucleotide encoding CCL19 or the polynucleotide encoding CCL19—the polynucleotide encoding IL-15.

In additional embodiments, a first vector (e.g., a first expression vector) comprises the polynucleotide encoding the CAR and either the polynucleotide encoding IL-15 or the polynucleotide encoding CCL19 and a second vector (e.g., a second expression vector) comprises the polynucleotide encoding IL-15 or the polynucleotide encoding CCL19 that is not included in the first vector. In some embodiments, the first vector (e.g., the first expression vector) comprises the polynucleotide encoding the CAR and the polynucleotide encoding IL-15 and the second vector (e.g., the second expression vector) comprises the polynucleotide encoding CCL19. In other embodiments, the first vector (e.g., the first expression vector) comprises the polynucleotide encoding the CAR and the polynucleotide encoding CCL19 and the second vector (e.g., the second expression vector) comprises the polynucleotide encoding IL-15.

In additional embodiments, a first vector (e.g., a first expression vector) comprises the polynucleotide encoding the CAR, a second vector (e.g., a second expression vector) comprises the polynucleotide encoding IL-15, and a third vector (e.g., a third expression vector) comprises the polynucleotide encoding CCL19.

In some embodiments, a vector (e.g., an expression vector) described herein comprises a polynucleotide encoding mIL-15/Ra and a polynucleotide encoding CCL19. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 14 (also referred to herein as mIL15/Ra-P2A-CCL19). In some cases, the nucleic acid sequence encodes a polypeptide comprising about 85% sequence identity to SEQ ID NO: 14. In some cases, the nucleic acid sequence encodes a polypeptide comprising about 90% sequence identity to SEQ ID NO: 14. In some cases, the nucleic acid sequence encodes a polypeptide comprising about 95% sequence identity to SEQ ID NO: 14. In some cases, the nucleic acid sequence encodes a polypeptide comprising about 96% sequence identity to SEQ ID NO: 14. In some cases, the nucleic acid sequence encodes a polypeptide comprising about 97% sequence identity to SEQ ID NO: 14. In some cases, the nucleic acid sequence encodes a polypeptide comprising about 98% sequence identity to SEQ ID NO: 14. In some cases, the nucleic acid sequence encodes a polypeptide comprising about 99% sequence identity to SEQ ID NO: 14. In some cases, the nucleic acid sequence encodes a polypeptide comprising SEQ ID NO: 14. In some cases, the nucleic acid sequence encodes a polypeptide consisting of SEQ ID NO: 14.

In some embodiments, a vector (e.g., an expression vector) described herein comprises a polynucleotide encoding sushi15 and a polynucleotide encoding CCL19. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 31 (also referred to herein as sushi15-CCL19). In some cases, the nucleic acid sequence encodes a polypeptide comprising about 85% sequence identity to SEQ ID NO: 31. In some cases, the nucleic acid sequence encodes a polypeptide comprising about 90% sequence identity to SEQ ID NO: 31. In some cases, the nucleic acid sequence encodes a polypeptide comprising about 95% sequence identity to SEQ ID NO: 31. In some cases, the nucleic acid sequence encodes a polypeptide comprising about 96% sequence identity to SEQ ID NO: 31. In some cases, the nucleic acid sequence encodes a polypeptide comprising about 97% sequence identity to SEQ ID NO: 31. In some cases, the nucleic acid sequence encodes a polypeptide comprising about 98% sequence identity to SEQ ID NO: 31. In some cases, the nucleic acid sequence encodes a polypeptide comprising about 99% sequence identity to SEQ ID NO: 31. In some cases, the nucleic acid sequence encodes a polypeptide comprising SEQ ID NO: 31. In some cases, the nucleic acid sequence encodes a polypeptide consisting of SEQ ID NO: 31.

In some embodiments, a vector (e.g., an expression vector) described herein comprises a polynucleotide encoding a CAR that specifically binds to mesothelin and a polynucleotide encoding mIL-15/Ra. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 34 (also referred to herein as ssV_{H}-P4-CAR-P2A-IL2sp-mIL15/Ra or P4-BB-mIL15/Ra). In some cases, the nucleic acid sequence encodes a polypeptide comprising about 85% sequence identity to SEQ ID NO: 34. In some cases, the nucleic acid sequence encodes a polypeptide comprising about 90% sequence identity to SEQ ID NO: 34. In some cases, the nucleic acid sequence encodes a polypeptide comprising about 95% sequence identity to SEQ ID NO: 34. In some cases, the nucleic acid sequence encodes a polypeptide comprising about 96% sequence identity to SEQ ID NO: 34. In some cases, the nucleic acid sequence encodes a polypeptide comprising about 97% sequence identity to SEQ ID NO: 34. In some cases, the nucleic acid sequence encodes a polypeptide comprising about 98% sequence identity to SEQ ID NO: 34. In some cases, the nucleic acid sequence encodes a polypeptide comprising about 99% sequence identity to SEQ ID NO: 34. In some cases, the nucleic acid sequence encodes a polypeptide comprising SEQ ID NO: 34. In some cases, the nucleic acid sequence encodes a polypeptide consisting of SEQ ID NO: 34.

In some embodiments, the vector comprising the nucleic acid sequence encoding ssV_{H}-P4-CAR-P2A-IL2sp-mIL15/Ra or P4-BB-mIL15/Ra comprises a sequence comprising about 80%, 85%, 90%, 95%, 96%, 97%, 98%, 99%, or 100% sequence identity to SEQ ID NO: 37. In some cases, the nucleic acid sequence comprises a sequence comprising about 85% sequence identity to SEQ ID NO: 37. In some cases, the nucleic acid sequence comprises a sequence comprising about 90% sequence identity to SEQ ID NO: 37. In some cases, the nucleic acid sequence comprises a sequence comprising about 95% sequence identity to SEQ ID NO: 37. In some cases, the nucleic acid sequence comprises a sequence comprising about 96% sequence identity to SEQ ID NO: 37. In some cases, the nucleic acid sequence comprises a sequence comprising about 97% sequence identity to SEQ ID NO: 37. In some cases, the nucleic acid sequence comprises a sequence comprising about 98% sequence identity to SEQ ID NO: 37. In some cases, the nucleic acid sequence comprises a sequence comprising about 99% sequence identity to SEQ ID NO: 37. In some cases, the nucleic acid sequence comprises SEQ ID NO: 37. In some cases, the nucleic acid sequence consists of SEQ ID NO: 37.

In some embodiments, a vector (e.g., an expression vector) described herein comprises a polynucleotide encoding a CAR that specifically binds to mesothelin and a polynucleotide encoding sushi15. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 35 (also referred to herein as ssV_{H}-P4-CAR-P2A-sushi15 or P4-BB-sushi15). In some cases, the nucleic acid sequence encodes a polypeptide comprising about 85% sequence identity to SEQ ID NO: 35. In some cases, the nucleic acid sequence encodes a polypeptide comprising about 90% sequence identity to SEQ ID NO: 35. In some cases, the nucleic acid sequence encodes a polypeptide comprising about 95% sequence identity to SEQ ID NO: 35. In some cases, the nucleic acid sequence encodes a polypeptide comprising about 96% sequence identity to SEQ ID NO: 35. In some cases, the nucleic acid sequence encodes a polypeptide comprising about 97% sequence identity to SEQ ID NO: 35. In some cases, the nucleic acid sequence encodes a polypeptide comprising about 98% sequence identity to SEQ ID NO: 35. In some cases, the nucleic acid sequence encodes a polypeptide comprising about 99% sequence identity to SEQ ID NO: 35. In some cases, the nucleic acid sequence encodes a polypeptide comprising SEQ ID NO: 35. In some cases, the nucleic acid sequence encodes a polypeptide consisting of SEQ ID NO: 35.

In some embodiments, the vector comprising the nucleic acid sequence encoding ssV_{H}-P4-CAR-P2A-sushi15 or P4-BB-sushi15 comprises a sequence comprising about 80%, 85%, 90%, 95%, 96%, 97%, 98%, 99%, or 100% sequence identity to SEQ ID NO: 38. In some cases, the nucleic acid sequence comprises a sequence comprising about 85% sequence identity to SEQ ID NO: 38. In some cases, the nucleic acid sequence comprises a sequence comprising about 90% sequence identity to SEQ ID NO: 38. In some cases, the nucleic acid sequence comprises a sequence comprising about 95% sequence identity to SEQ ID NO: 38. In some cases, the nucleic acid sequence comprises a sequence comprising about 96% sequence identity to SEQ ID NO: 38. In some cases, the nucleic acid sequence comprises a sequence comprising about 97% sequence identity to SEQ ID NO: 38. In some cases, the nucleic acid sequence comprises a sequence comprising about 98% sequence identity to SEQ ID NO: 38. In some cases, the nucleic acid sequence comprises a sequence comprising about 99% sequence identity to SEQ ID NO: 38. In some cases, the nucleic acid sequence comprises SEQ ID NO: 38. In some cases, the nucleic acid sequence consists of SEQ ID NO: 38.

In some embodiments, a vector (e.g., an expression vector) described herein comprises a polynucleotide encoding a CAR that specifically binds to mesothelin, a polynucleotide encoding mIL-15/Ra and a polynucleotide encoding CCL19. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 36 (also referred to herein as ssV_{H}-P4-CAR-P2A-IL2sp-mIL15/Ra-P2A-CCL19 or P4-BB-mIL15-CCL19). In some cases, the nucleic acid sequence encodes a polypeptide comprising about 85% sequence identity to SEQ ID NO: 36. In some cases, the nucleic acid sequence encodes a polypeptide comprising about 90% sequence identity to SEQ ID NO: 36. In some cases, the nucleic acid sequence encodes a polypeptide comprising about 95% sequence identity to SEQ ID NO: 36. In some cases, the nucleic acid sequence encodes a polypeptide comprising about 96% sequence identity to SEQ ID NO: 36. In some cases, the nucleic acid sequence encodes a polypeptide comprising about 97% sequence identity to SEQ ID NO: 36. In some cases, the nucleic acid sequence encodes a polypeptide comprising about 98% sequence identity to SEQ ID NO: 36. In some cases, the nucleic acid sequence encodes a polypeptide comprising about 99% sequence identity to SEQ ID NO: 36. In some cases, the nucleic acid sequence encodes a polypeptide comprising SEQ ID NO: 36. In some cases, the nucleic acid sequence encodes a polypeptide consisting of SEQ ID NO: 36.

In some embodiments, the vector comprising the nucleic acid sequence encoding ssV_{H}-P4-CAR-P2A-IL2sp-mIL15/Ra-P2A-CCL19 or P4-BB-mIL15-CCL19 comprises a sequence comprising about 80%, 85%, 90%, 95%, 96%, 97%, 98%, 99%, or 100% sequence identity to SEQ ID NO: 39. In some cases, the nucleic acid sequence comprises a sequence comprising about 85% sequence identity to SEQ ID NO: 39. In some cases, the nucleic acid sequence comprises a sequence comprising about 90% sequence identity to SEQ ID NO: 39. In some cases, the nucleic acid sequence comprises a sequence comprising about 95% sequence identity to SEQ ID NO: 39. In some cases, the nucleic acid sequence comprises a sequence comprising about 96% sequence identity to SEQ ID NO: 39. In some cases, the nucleic acid sequence comprises a sequence comprising about 97% sequence identity to SEQ ID NO: 39. In some cases, the nucleic acid sequence comprises a sequence comprising about 98% sequence identity to SEQ ID NO: 39. In some cases, the nucleic acid sequence comprises a sequence comprising about 99% sequence identity to SEQ ID NO: 39. In some cases, the nucleic acid sequence comprises SEQ ID NO: 39. In some cases, the nucleic acid sequence consists of SEQ ID NO: 39.

In some embodiments, a vector (e.g., an expression vector) described herein comprises a polynucleotide encoding a CAR that specifically binds to mesothelin, a polynucleotide encoding sushi15 and a polynucleotide encoding CCL19. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 15 (also referred to herein as ssV_{H}-P4-CAR-P2A-sushi15-P2A-CCL19 or P4-BB-sushi15-CCL19). In some cases, the nucleic acid sequence encodes a polypeptide comprising about 85% sequence identity to SEQ ID NO: 15. In some cases, the nucleic acid sequence encodes a polypeptide comprising about 90% sequence identity to SEQ ID NO: 15. In some cases, the nucleic acid sequence encodes a polypeptide comprising about 95% sequence identity to SEQ ID NO: 15. In some cases, the nucleic acid sequence encodes a polypeptide comprising about 96% sequence identity to SEQ ID NO: 15. In some cases, the nucleic acid sequence encodes a polypeptide comprising about 97% sequence identity to SEQ ID NO: 15. In some cases, the nucleic acid sequence encodes a polypeptide comprising about 98% sequence identity to SEQ ID NO: 15. In some cases, the nucleic acid sequence encodes a polypeptide comprising about 99% sequence identity to SEQ ID NO: 15. In some cases, the nucleic acid sequence encodes a polypeptide comprising SEQ ID NO: 15. In some cases, the nucleic acid sequence encodes a polypeptide consisting of SEQ ID NO: 15.

In some embodiments, the vector comprising the nucleic acid sequence encoding ssV_{H}-P4-CAR-P2A-sushi15-P2A-CCL19 or P4-BB-sushi15-CCL19 comprises a sequence comprising about 80%, 85%, 90%, 95%, 96%, 97%, 98%, 99%, or 100% sequence identity to SEQ ID NO: 40. In some cases, the nucleic acid sequence comprises a sequence comprising about 85% sequence identity to SEQ ID NO: 40. In some cases, the nucleic acid sequence comprises a sequence comprising about 90% sequence identity to SEQ ID NO: 40. In some cases, the nucleic acid sequence comprises a sequence comprising about 95% sequence identity to SEQ ID NO: 40. In some cases, the nucleic acid sequence comprises a sequence comprising about 96% sequence identity to SEQ ID NO: 40. In some cases, the nucleic acid sequence comprises a sequence comprising about 97% sequence identity to SEQ ID NO: 40. In some cases, the nucleic acid sequence comprises a sequence comprising about 98% sequence identity to SEQ ID NO: 40. In some cases, the nucleic acid sequence comprises a sequence comprising about 99% sequence identity to SEQ ID NO: 40. In some cases, the nucleic acid sequence comprises SEQ ID NO: 40. In some cases, the nucleic acid sequence consists of SEQ ID NO: 40.

In some embodiments, a vector (e.g., an expression vector) described herein comprises a polynucleotide encoding a CAR that specifically binds to mesothelin and a polynucleotide encoding mIL15/Ra-LSP. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 53 (also referred to herein as ssVHsp-P4-CD8h/TM-BB-CD3z-P2A-IL15LSP-IL15propeptide-mIL15/Ra or SSVHsp-P4-BB-mIL15/Ra-LSP). In some cases, the nucleic acid sequence encodes a polypeptide comprising about 85% sequence identity to SEQ ID NO: 53. In some cases, the nucleic acid sequence encodes a polypeptide comprising about 90% sequence identity to SEQ ID NO: 53. In some cases, the nucleic acid sequence encodes a polypeptide comprising about 95% sequence identity to SEQ ID NO: 53. In some cases, the nucleic acid sequence encodes a polypeptide comprising about 96% sequence identity to SEQ ID NO: 53. In some cases, the nucleic acid sequence encodes a polypeptide comprising about 97% sequence identity to SEQ ID NO: 53. In some cases, the nucleic acid sequence encodes a polypeptide comprising about 98% sequence identity to SEQ ID NO: 53. In some cases, the nucleic acid sequence encodes a polypeptide comprising about 99% sequence identity to SEQ ID NO: 53. In some cases, the nucleic acid sequence encodes a polypeptide comprising SEQ ID NO: 53. In some cases, the nucleic acid sequence encodes a polypeptide consisting of SEQ ID NO: 53.

In some embodiments, the vector comprising the nucleic acid sequence encoding ssVHsp-P4-CD8h/TM-BB-CD3z-P2A-IL15LSP-IL15propeptide-mIL15/Ra or SSVHsp-P4-BB-mIL15/Ra-LSP comprises a sequence comprising about 80%, 85%, 90%, 95%, 96%, 97%, 98%, 99%, or 100% sequence identity to SEQ ID NO: 58. In some cases, the nucleic acid sequence comprises a sequence comprising about 85% sequence identity to SEQ ID NO: 58. In some cases, the nucleic acid sequence comprises a sequence comprising about 90% sequence identity to SEQ ID NO: 58. In some cases, the nucleic acid sequence comprises a sequence comprising about 95% sequence identity to SEQ ID NO: 58. In some cases, the nucleic acid sequence comprises a sequence comprising about 96% sequence identity to SEQ ID NO: 58. In some cases, the nucleic acid sequence comprises a sequence comprising about 97% sequence identity to SEQ ID NO: 58. In some cases, the nucleic acid sequence comprises a sequence comprising about 98% sequence identity to SEQ ID NO: 58. In some cases, the nucleic acid sequence comprises a sequence comprising about 99% sequence identity to SEQ ID NO: 58. In some cases, the nucleic acid sequence comprises SEQ ID NO: 58. In some cases, the nucleic acid sequence consists of SEQ ID NO: 58.

In some embodiments, a vector (e.g., an expression vector) described herein comprises a polynucleotide encoding a CAR that specifically binds to mesothelin and a polynucleotide encoding sIL15-LSP. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 54 (also referred to herein as ssVHsp-P4-CD8h/TM-BB-CD3z-P2A-IL15LSP-IL 15propeptide-IL15 or SSVHsp-P4-BB-sIL15-LSP). In some cases, the nucleic acid sequence encodes a polypeptide comprising about 85% sequence identity to SEQ ID NO: 54. In some cases, the nucleic acid sequence encodes a polypeptide comprising about 90% sequence identity to SEQ ID NO: 54. In some cases, the nucleic acid sequence encodes a polypeptide comprising about 95% sequence identity to SEQ ID NO: 54. In some cases, the nucleic acid sequence encodes a polypeptide comprising about 96% sequence identity to SEQ ID NO: 54. In some cases, the nucleic acid sequence encodes a polypeptide comprising about 97% sequence identity to SEQ ID NO: 54. In some cases, the nucleic acid sequence encodes a polypeptide comprising about 98% sequence identity to SEQ ID NO: 54. In some cases, the nucleic acid sequence encodes a polypeptide comprising about 99% sequence identity to SEQ ID NO: 54. In some cases, the nucleic acid sequence encodes a polypeptide comprising SEQ ID NO: 54. In some cases, the nucleic acid sequence encodes a polypeptide consisting of SEQ ID NO: 54.

In some embodiments, the vector comprising the nucleic acid sequence encoding ssVHsp-P4-CD8h/TM-BB-CD3z-P2A-IL15LSP-IL15propeptide-IL15 or SSVHsp-P4-BB-sIL15-LSP comprises a sequence comprising about 80%, 85%, 90%, 95%, 96%, 97%, 98%, 99%, or 100% sequence identity to SEQ ID NO: 59. In some cases, the nucleic acid sequence comprises a sequence comprising about 85% sequence identity to SEQ ID NO: 59. In some cases, the nucleic acid sequence comprises a sequence comprising about 90% sequence identity to SEQ ID NO: 59. In some cases, the nucleic acid sequence comprises a sequence comprising about 95% sequence identity to SEQ ID NO: 59. In some cases, the nucleic acid sequence comprises a sequence comprising about 96% sequence identity to SEQ ID NO: 59. In some cases, the nucleic acid sequence comprises a sequence comprising about 97% sequence identity to SEQ ID NO: 59. In some cases, the nucleic acid sequence comprises a sequence comprising about 98% sequence identity to SEQ ID NO: 59. In some cases, the nucleic acid sequence comprises a sequence comprising about 99% sequence identity to SEQ ID NO: 59. In some cases, the nucleic acid sequence comprises SEQ ID NO: 59. In some cases, the nucleic acid sequence consists of SEQ ID NO: 59.

In some embodiments, a vector (e.g., an expression vector) described herein comprises a polynucleotide encoding a CAR that specifically binds to mesothelin, a polynucleotide encoding mIL15/Ra-LSP and a polynucleotide encoding CCL19. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 55 (also referred to herein as ssVHsp-P4-CD8h/TM-BB-CD3z-P2A-IL15LSP-IL15propeptide-IL15-P2A-endospCCL19 or ssVHsp-P4-BB-mIL15/Ra-LSP-CCL19). In some cases, the nucleic acid sequence encodes a polypeptide comprising about 85% sequence identity to SEQ ID NO: 55. In some cases, the nucleic acid sequence encodes a polypeptide comprising about 90% sequence identity to SEQ ID NO: 55. In some cases, the nucleic acid sequence encodes a polypeptide comprising about 95% sequence identity to SEQ ID NO: 55. In some cases, the nucleic acid sequence encodes a polypeptide comprising about 96% sequence identity to SEQ ID NO: 55. In some cases, the nucleic acid sequence encodes a polypeptide comprising about 97% sequence identity to SEQ ID NO: 55. In some cases, the nucleic acid sequence encodes a polypeptide comprising about 98% sequence identity to SEQ ID NO: 55. In some cases, the nucleic acid sequence encodes a polypeptide comprising about 99% sequence identity to SEQ ID NO: 55. In some cases, the nucleic acid sequence encodes a polypeptide comprising SEQ ID NO: 55. In some cases, the nucleic acid sequence encodes a polypeptide consisting of SEQ ID NO: 55.

In some embodiments, the vector comprising the nucleic acid sequence encoding ssVHsp-P4-CD8h/TM-BB-CD3z-P2A-IL15LSP-IL15propeptide-IL15-P2A-endospCCL19 or ssVHsp-P4-BB-mIL15/Ra-LSP-CCL19 comprises a sequence comprising about 80%, 85%, 90%, 95%, 96%, 97%, 98%, 99%, or 100% sequence identity to SEQ ID NO: 60. In some cases, the nucleic acid sequence comprises a sequence comprising about 85% sequence identity to SEQ ID NO: 60. In some cases, the nucleic acid sequence comprises a sequence comprising about 90% sequence identity to SEQ ID NO: 60. In some cases, the nucleic acid sequence comprises a sequence comprising about 95% sequence identity to SEQ ID NO: 60. In some cases, the nucleic acid sequence comprises a sequence comprising about 96% sequence identity to SEQ ID NO: 60. In some cases, the nucleic acid sequence comprises a sequence comprising about 97% sequence identity to SEQ ID NO: 60. In some cases, the nucleic acid sequence comprises a sequence comprising about 98% sequence identity to SEQ ID NO: 60. In some cases, the nucleic acid sequence comprises a sequence comprising about 99% sequence identity to SEQ ID NO: 60. In some cases, the nucleic acid sequence comprises SEQ ID NO: 60. In some cases, the nucleic acid sequence consists of SEQ ID NO: 60.

In some embodiments, a vector (e.g., an expression vector) described herein comprises a polynucleotide encoding a CAR that specifically binds to mesothelin, a polynucleotide encoding sIL15-LSP and a polynucleotide encoding CCL19. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 56 (also referred to herein as ssVHsp-P4-CD8h/TM-BB-CD3z-P2A-IL15LSP-IL15propeptide-IL15-P2A-endospCCL19 or ssVHsp-P4-BB-sIL15-LSP-CCL19). In some cases, the nucleic acid sequence encodes a polypeptide comprising about 85% sequence identity to SEQ ID NO: 56. In some cases, the nucleic acid sequence encodes a polypeptide comprising about 90% sequence identity to SEQ ID NO: 56. In some cases, the nucleic acid sequence encodes a polypeptide comprising about 95% sequence identity to SEQ ID NO: 56. In some cases, the nucleic acid sequence encodes a polypeptide comprising about 96% sequence identity to SEQ ID NO: 56. In some cases, the nucleic acid sequence encodes a polypeptide comprising about 97% sequence identity to SEQ ID NO: 56. In some cases, the nucleic acid sequence encodes a polypeptide comprising about 98% sequence identity to SEQ ID NO: 56. In some cases, the nucleic acid sequence encodes a polypeptide comprising about 99% sequence identity to SEQ ID NO: 56. In some cases, the nucleic acid sequence encodes a polypeptide comprising SEQ ID NO: 56. In some cases, the nucleic acid sequence encodes a polypeptide consisting of SEQ ID NO: 56.

In some embodiments, the vector comprising the nucleic acid sequence encoding ssVHsp-P4-CD8h/TM-BB-CD3z-P2A-IL15LSP-IL15propeptide-IL15-P2A-endospCCL19 or ssVHsp-P4-BB-sIL15-LSP-CCL19 comprises a sequence comprising about 80%, 85%, 90%, 95%, 96%, 97%, 98%, 99%, or 100% sequence identity to SEQ ID NO: 61. In some cases, the nucleic acid sequence comprises a sequence comprising about 85% sequence identity to SEQ ID NO: 61. In some cases, the nucleic acid sequence comprises a sequence comprising about 90% sequence identity to SEQ ID NO: 61. In some cases, the nucleic acid sequence comprises a sequence comprising about 95% sequence identity to SEQ ID NO: 61. In some cases, the nucleic acid sequence comprises a sequence comprising about 96% sequence identity to SEQ ID NO: 61. In some cases, the nucleic acid sequence comprises a sequence comprising about 97% sequence identity to SEQ ID NO: 61. In some cases, the nucleic acid sequence comprises a sequence comprising about 98% sequence identity to SEQ ID NO: 61. In some cases, the nucleic acid sequence comprises a sequence comprising about 99% sequence identity to SEQ ID NO: 61. In some cases, the nucleic acid sequence comprises SEQ ID NO: 61. In some cases, the nucleic acid sequence consists of SEQ ID NO: 61.

In some embodiments, a vector (e.g., an expression vector) described herein comprises a polynucleotide encoding a CAR that specifically binds to mesothelin, a polynucleotide encoding sushi15 and a polynucleotide encoding CCL19. In some instances, the vector (e.g., the expression vector) comprises a nucleic acid sequence encoding a polypeptide comprising about 80%, 85%, 90%, 95%, 96%, 97%, 98%, 99%, or 100% sequence identity to SEQ ID NO: 57 (also referred to herein as ssV_{H}-P4-CD28hinge-TM-CD28cyto-CD3z-P2A-IL15Rasp-IL15Rα (sushi)-20aalinker-IL15-P2A-endospCCL19 or ssVHsp-P4-CD28-sushi15-CCL19). In some cases, the nucleic acid sequence encodes a polypeptide comprising about 85% sequence identity to SEQ ID NO: 57. In some cases, the nucleic acid sequence encodes a polypeptide comprising about 90% sequence identity to SEQ ID NO: 57. In some cases, the nucleic acid sequence encodes a polypeptide comprising about 95% sequence identity to SEQ ID NO: 57. In some cases, the nucleic acid sequence encodes a polypeptide comprising about 96% sequence identity to SEQ ID NO: 57. In some cases, the nucleic acid sequence encodes a polypeptide comprising about 97% sequence identity to SEQ ID NO: 57. In some cases, the nucleic acid sequence encodes a polypeptide comprising about 98% sequence identity to SEQ ID NO: 57. In some cases, the nucleic acid sequence encodes a polypeptide comprising about 99% sequence identity to SEQ ID NO: 57. In some cases, the nucleic acid sequence encodes a polypeptide comprising SEQ ID NO: 57. In some cases, the nucleic acid sequence encodes a polypeptide consisting of SEQ ID NO: 57.

Vectors (e.g., expression vectors) of the present invention may comprise one or more naturally derived nucleic acids or artificially synthesized nucleic acids, and can be appropriately selected according to the type of cells to which the vectors (e.g., the expression vectors) of the present invention are to be introduced. Their sequence information can be appropriately obtained by the search of a publicly known document or a database such as NCBI (www.ncbi.nlm.nih.gov/guide/).

The vector of the present invention can be an expression vector that is introduced into an immune cell or its precursor cell by contacting the vector with the cell so that a predetermined protein (polypeptide) encoded therein can be expressed in the immune cell to produce the modified immune cell of the present invention. The expression vector of the present invention is not particularly limited by any embodiment. Those skilled in the art are capable of designing and producing an expression vector that permits expression of the desired protein (polypeptide) in immune cells. Examples of the expression vector of the present invention comprising a polynucleotide encoding a cell surface molecule specifically recognizing human mesothelin, a polynucleotide encoding IL-15, and optionally a polynucleotide encoding CCL19 can include any of expression vectors for producing the immune cell of the present invention.

The type of expression vector of the present invention may be a linear form or a circular form and may be a non-viral vector such as a plasmid, may be a viral vector, or may be a vector based on a transposon. Such vector may contain a control sequence such as a promoter or a terminator, or a selective marker sequence such as a drug resistance gene or a reporter gene. The polynucleotide encoding IL-15 and the polynucleotide encoding CCL19 can be operably arranged downstream of the promoter sequence so that each of the polynucleotides can be efficiently transcribed.

Examples of the promoter can include: a virus-derived promoter such as retrovirus LTR promoter, SV40 early promoter, cytomegalovirus promoter, and herpes simplex virus thymidine kinase promoter; and a mammal-derived promoter such as phosphoglycerate kinase (PGK) promoter, Xist promoter, β-actin promoter, and RNA polymerase II promoter. In some embodiment, the promoter can preferably include retrovirus LTR promoter. The retrovirus LTR promoter can comprise CTGAATATGGGCCAAACAGGATATCTGTGGTAAGCAGTTCCTGCCCCGGCT CAGGGCCAAGAACAGATGGAACAGCTGAATATGGGCCAAACAGGATATCT GTGGTAAGCAGTTCCTGCCCCGGCTCAGGGCCAAGAACAGATGGTCCCCAG ATGCGGTCCAGCCCTCAGCAGTTTCTAGAGAACCATCAGATGTTTCCAGGGT GCCCCAAGGACCTGAAATGACCCTGTGCCTTATTTGAACTAACCAATCAGTT CGCTTCTCGCTTCTGTTCGCGCGCTTCTGCTCCCCGAGCTCAATAAAAGAGC CCACAACCCCTCACTCGGCGCGCCAGTCCTCCGATTGACTGAGTCGCCCGGG TACCCGTGTATCCAATAAACCCTCTTGCAGTTGCATCCGACTTGTGGTCTCG CTGTTCCTTGGGAGGGTCTCCTCTGAGTGATTGACTACCCGTCAGCGGGGGT CTTTCA (SEQ ID NO: 81). Alternatively, tetracycline-responsive promoter which is induced by tetracycline, M×1 promoter which is induced by interferon, or the like may be used. Use of the promoter which is induced by a particular substance in the expression vector of the present invention permits control of induction of IL-15 expression and optionally CCL19 expression according to the course of treatment of cancer, for example, when the immune cell containing the vector of the present invention is used as a pharmaceutical composition for use in the treatment of cancer.

Examples of the viral vector can include a retrovirus vector, a lentivirus vector, an adenovirus vector, and an adeno-associated virus vector and can preferably include a retrovirus vector, preferably a gamma retrovirus vector, more preferably a pMSGV vector (Tamada k et al., Clin Cancer Res 18:6436-6445 (2002)), a pMSCV vector (manufactured by Takara Bio Inc.), or a pSFG vector. Use of a retrovirus vector permits long-term and stable expression of a transgene because the transgene is integrated in the genome of a host cell.

One or more assays can be used to confirm the containment of the expression vector of the present invention in the immune cell. Exemplary assays can include flow cytometry for screening the expression of CAR by the engineered immune cells, Northern blotting, Southern blotting, PCR such as RT-PCR, ELISA, or Western blotting. In some embodiments, the expression vector further comprises a marker gene (e.g., encoding a fluorescent protein such as green fluorescent protein (GFP), red fluorescent protein (RFP), or yellow fluorescent protein (YFP)) to detect the expression of the CAR, IL-15, and/or CCL19 by the immune cell.

### Immune Cells and Methods of Production

In certain embodiments, an immune cell described herein is modified to express a cell surface molecule that specifically recognizes mesothelin (e.g., human mesothelin), IL-15, and optionally CCL19 (FIG. 1C and FIG. 1D). Exemplary immune cells can include a lymphoid cell such as a T cell, a natural killer cell (NK cell), and a B cell, an antigen presenting cell such as a monocyte, a macrophage, a dendritic cell, or a granulocyte such as a neutrophil, an eosinophil, a basophil, or a mast cell. The immune cells can be derived from a stem cell, e.g., an induced pluripotent stem cell (iPSC), an embryonic stem cell (ESC), ESC-derived hematopoietic progenitor cell (HPC), or hemogenic endothelial cell (HEC). The immune cell can include a T cell derived from a mammal such as a human, a dog, a cat, a pig, or a mouse, preferably a T cell derived or separated from a human. The immune cell (e.g., a T cell) can be obtained through culturing, e.g., ex vivo culturing, or harvested directly from the mammal. The immune cell is not limited so long as the cell is involved in immune response and can express the cell surface molecule that specifically recognizes mesothelin (e.g., human mesothelin), expresses IL-15, and optionally expresses CCL19. The immune cell can be an autologous cell harvested from a subject in need thereof for subsequent treatment. The immune cell can also be an allogeneic cell or a syngeneic cell, to a subject in need thereof.

In some embodiments, the immune cell is further modified to impair or ameliorate an inflammation or an immune response in the subject due to administration of the immune cell in the subject. In some instances, the inflammation or immune response is Graft versus Host Disease (GvHD) and the immune cell is further modified to impair or ameliorate GvHD.

In some cases, the immune cell is modified to impair or remove the expression of the alpha chain and/or the beta chain of T-cell receptor (TCR). In some cases, the immune cell is modified to impair or remove the expression of the alpha chain and the beta chain of TCR (TCRα/β). In some cases, the immune cell does not express the alpha chain and the beta chain of TCR (TCRα/β). In some cases, one or more of the additional CD3 domains (e.g., CD3 delta, CD3 gamma, or CD3 epsilon) are modified to impair TCRα/β function.

In some instances, the immune cell expresses a gamma delta T cell receptor (gdTCR or γδTCR). γδT cells are a subgroup of T cells and accounts for about 0.5-5% of all T-lymphocytes. Under a cancer setting, γδT cells have been shown to exert both protumor and anti-tumor activities, based on the combinatorial subgroups of γ and 8 receptors presented at the surface of the cell. For example, a γδT cell expressing a TCR containing the γ-chain variable region 9 (Vγ9) and the δ-chain variable region 2 (Vδ2), also referred to as Vγ9Vδ2 T cells, exert anti-tumor activity, including but not limited to inhibiting cancer cell proliferation, angiogenesis, lymphangiogenesis, and increase cancer cell apoptosis. Examples of the γTCR include Vγ1TCR, Vγ2TCR, Vγ3TCR, Vγ4TCR, Vγ5TCR, Vγ6TCR, Vγ7TCR, Vγ8TCR, and Vγ9TCR, and examples of the δTCR include Vδ1TCR, Vδ2TCR, Vδ3TCR, Vδ4TCR, Vδ5TCR, Vδ6TCR, Vδ7TCR, Vδ8TCR, and Vδ9TCR. While the combination of specific γTCR and δTCR is not limited, for example, Vγ3Vδ1TCR, Vγ4Vδ1TCR, Vγ9Vδ1TCR, and Vγ9Vδ2TCR are contemplated. In some cases, the immune cell expresses Vγ9 TCR, Vδ2 TCR, or Vγ9Vδ2 TCR.

In some embodiments, an immune cell described herein expressing a gamma delta T cell receptor (gdTCR or γδTCR) is produced from induced pluripotent stem cells (iPSCs). In some instances, the iPSCs are established from a cell other than an αβT cell. In some instances, the iPSC is further modified by inserting the Vγ9Vδ2 gene into the genome of the iPSC prior to differentiating into γδTCR cells. In some instances, the Vγ9Vδ2 gene encodes a polypeptide comprising at least 90%, 91%, 92%, 93%, 94%, 95%, 96%, 97%, 98%, 99%, or 100% sequence identity to SEQ ID NO: 44. In some instances, the Vγ9Vδ2 gene encodes a polypeptide consisting of SEQ ID NO: 44.

In some embodiments, a γδTCR cell disclosed herein is modified to express a CAR that specifically recognizes mesothelin and to express IL-15. In some cases, the γδTCR cell is further modified to express CCL19. In some cases, the γδTCR cell is differentiated from an iPSC modified with a Vγ9Vδ2 gene. In some cases, the γδTCR cell is differentiated from an iPSC modified with a Vγ9Vδ2 gene encoding a polypeptide comprising at least 90%, 91%, 92%, 93%, 94%, 95%, 96%, 97%, 98%, 99%, or 100% sequence identity to SEQ ID NO: 44. In some cases, the γδTCR cell is differentiated from an iPSC modified with a Vγ9Vδ2 gene encoding a polypeptide consisting of SEQ ID NO: 44.

In certain embodiments, disclosed herein is a population of immune cells modified to express a CAR that specifically recognizes mesothelin and IL-15. In some cases, the population of immune cells further expresses CCL19. In some embodiments, the population of immune cells comprises modified T cells (e.g., either expanded ex vivo or harvested from a mammal) that express a CAR that specifically recognizes mesothelin, IL-15, and optionally CCL19. The population of immune cells can comprise about 20%, 30%, 40%, 50%, 60%, 70%, 80%, 90%, 95%, or higher percentage of the modified T cells. The population of immune cells can comprise about 20%, 30%, 40%, 50%, 60%, 70%, 80%, 90%, or 95% modified T cells. The population of immune cells can comprise a substantially pure population of modified T cells. Exemplary T cells can include an alpha-beta T cell, a gamma-delta T cell, a CD8 T cell, a CD4 T cell, a tumor infiltrating T cell, a memory T cell, a naive T cell, and a natural killer T (NKT) cell.

In some embodiments, the population of immune cells modified to express a CAR that specifically recognizes mesothelin, IL-15, and optionally CCL19 comprises less than about 30%, 25%, 20%, 15%, 10%, 5%, or less contaminant cells. As used herein, the term “contaminant cells” refer to cells that do not express a CAR that specifically recognizes mesothelin, IL-15, and optionally CCL19. The contaminant cells can include T cells that do not express a CAR that specifically recognizes mesothelin, IL-15, and optionally CCL19, and other type of immune cells that do not express a CAR that specifically recognizes mesothelin, IL-15, and optionally CCL19. The contaminant cells can also refer to non-immune cells from a body fluid such as blood or bone marrow fluid, derived from a tissue such as a spleen tissue, the thymus gland, or a lymph node, or derived from a cancer tissue such as a primary tumor tissue, metastatic tumor tissue, or cancerous ascites.

Examples of the method for producing the immune cell of the present invention can include a production method of introducing a polynucleotide encoding a cell surface molecule, a polynucleotide encoding IL-15, and optionally a polynucleotide encoding CCL19 to an immune cell. The production method can include a production method as described in, for example, WO2016/056228, WO2017/159736, WO2013/176915, WO2015/120096, WO2016/019300, or Vormittag P et al, Curr Opin Biotechnol 2018; 53:164-81. Alternative examples can include a method of purifying and obtaining an immune cell from a transgenic mammal produced by implanting a vector for expression of a cell surface molecule specifically recognizing mesothelin (e.g., human mesothelin) and/or IL-15, and further optionally CCL19 into a fertilized egg, and a production method of further introducing, if necessary, the vector for expression of a cell surface molecule specifically recognizing mesothelin (e.g., human mesothelin) and/or IL-15, and further optionally CCL19 to the immune cell purified and obtained from the transgenic mammal.

In the case of introducing a polynucleotide encoding a cell surface molecule, a polynucleotide encoding IL-15, and optionally a polynucleotide encoding CCL19, or the vectors described supra, the method can be any method for introducing the polynucleotides or the vectors to the immune cell. Examples can include an electroporation method (Cytotechnology, 3, 133 (1990)), a calcium phosphate method (Japanese unexamined Patent Application Publication No. 2-227075), a lipofection method (Proc. Natl. Acad. Sci. U.S.A., 84, 7413 (1987)), and a viral infection method. Exemplary viral infection methods can include a method of transfecting a packaging cell such as a GP2-293 cell (manufactured by Takara Bio Inc.), a Plat-GP cell (manufactured by Cosmo Bio Co., Ltd.), a PG13 cell (ATCC CRL-10686), or a PA317 cell (ATCC CRL-9078) with the vector to be introduced and a packaging plasmid to produce a recombinant virus, and infecting the immune cell with the recombinant virus (see e.g., WO2017/159736).

In some embodiments, the method comprises introducing one or more vectors comprising the polynucleotide encoding the CAR, the polynucleotide encoding IL-15, and optionally the polynucleotide encoding CCL19 to an immune cell. In some embodiments, the method comprises introducing a vector (e.g., an expression vector) comprising the nucleic acid molecule comprising a polynucleotide encoding a chimeric antigen receptor (CAR) comprising an antibody that specifically recognizes human mesothelin, a CD8 hinge region, a CD8 transmembrane region, a 4-1BB intracellular region, and a CD3 (intracellular region; a polynucleotide encoding IL-15; and optionally a polynucleotide encoding CCL19 to an immune cell. In some embodiments, the method comprises introducing a first vector (e.g., a first expression vector) comprising the polynucleotide encoding the CAR and a second vector (e.g., a second expression vector) comprising the polynucleotide encoding IL-15, either together or in stages, to an immune cell. In some embodiments, the method comprises introducing a first vector (e.g., a first expression vector) comprising the polynucleotide encoding the CAR and a second vector (e.g., a second expression vector) comprising the polynucleotide encoding IL-15 and the polynucleotide encoding CCL19, either together or in stages, to an immune cell. In some embodiments, the method comprises introducing a first vector (e.g., a first expression vector) comprising the polynucleotide encoding the CAR and either the polynucleotide encoding IL-15 or the polynucleotide encoding CCL19 and a second vector (e.g., a second expression vector) comprising the polynucleotide encoding IL-15 or the polynucleotide encoding CCL19 that is not included in the first vector, either together or in stages, to an immune cell. In some embodiments, the method comprises introducing a first vector (e.g., a first expression vector) comprising the polynucleotide encoding the CAR and the polynucleotide encoding IL-15 and a second vector (e.g., a second expression vector) comprising the polynucleotide encoding CCL19, either together or in stages, to an immune cell. In some embodiments, the method comprises introducing a first vector (e.g., a first expression vector) comprising the polynucleotide encoding the CAR and the polynucleotide encoding CCL19 and a second vector (e.g., a second expression vector) comprising the polynucleotide encoding IL-15, either together or in stages, to an immune cell. In some embodiments, the method comprises introducing a first vector (e.g., a first expression vector) comprising the polynucleotide encoding the CAR, a second vector (e.g., a second expression vector) comprising the polynucleotide encoding IL-15, and a third vector (e.g., a third expression vector) comprising the polynucleotide encoding CCL19, either together or in stages, to an immune cell.

One or more of the polynucleotide encoding the CAR, the polynucleotide encoding IL-15, and the polynucleotide encoding CCL19 can be integrated into the genome of the immune cell. In some embodiments, the polynucleotide encoding the CAR, the polynucleotide encoding IL-15, and the polynucleotide encoding CCL19 are not integrated into the genome (e.g., episomally).

### Methods of Use

In certain embodiments, disclosed herein is a method of treating a mesothelin-expressing cancer. In some embodiments, the method comprises administering to a subject in need thereof an immune cell described herein modified to express an engineered cell surface molecule that specifically binds to mesothelin, interleukin 15 (IL-15), and optionally chemokine (C-C motif) ligand 19 (CCL19). In some embodiments, the immune cell is modified to express an engineered cell surface molecule comprises a chimeric antigen receptor (CAR) that specifically recognizes mesothelin or a T cell receptor (TCR) that specifically binds to mesothelin. In some embodiments, the immune cell is modified to express a CAR comprising an antibody that specifically recognizes human mesothelin, a CD8 hinge region, a CD8 transmembrane region, a 4-1BB intracellular region and a CD3ζintracellular region; IL-15; and optionally CCL19.

In some embodiments, the mesothelin-expressing cancer is a solid tumor. In some embodiments, the solid tumor comprises mesothelioma, colorectal cancer, pancreatic cancer, thymic cancer, bile duct cancer, lung cancer, skin cancer, breast cancer, prostate cancer, urinary bladder cancer, virginal cancer, neck cancer, uterine cancer, liver cancer, kidney cancer, gastric cancer, spleen cancer, tracheal cancer, bronchial cancer, stomach cancer, esophageal cancer, gallbladder cancer, testis cancer, ovarian cancer, or bone cancer. In some embodiments, the mesothelin-expressing cancer is ovarian cancer. In some embodiments, the mesothelin-expressing cancer is mesothelioma. In some embodiments, the mesothelin-expressing cancer is gastric cancer. In some embodiments, the mesothelin-expressing cancer is lung cancer (e.g., non-small cell lung cancer (NSCLC), small cell lung cancer (SCLC), lung carcinoid tumors, adenosquamous carcinoma of the lung, large cell neuroendocrine carcinoma, or salivary gland-type lung carcinoma). In some embodiments, the mesothelin-expressing cancer is NSCLC (e.g., adenocarcinoma of the lung, squamous cell, large-cell undifferentiated carcinoma, sarcomatoid carcinoma, or adenosquamous carcinoma).

The mesothelin-expressing cancer can be a hematopoietic cancer. The hematopoietic cancer can be a B-cell hematopoietic cancer, a T-cell hematopoietic cancer, a Hodgkin's lymphoma, or a non-Hodgkin's lymphoma. The hematopoietic cancer can be acute lymphocytic leukemia (ALL), chronic lymphocytic leukemia (CLL), small lymphocytic lymphoma (SLL), diffuse large B-cell lymphoma (DLBCL), follicular lymphoma (FL), mantle cell lymphoma (MCL), marginal zone lymphomas, Burkitt lymphoma, or Waldenstrom macroglobulinemia.

The hematopoietic cancer can be a sarcoma. The sarcoma can include chondrosarcoma, Ewing's sarcoma, malignant hemangioendothelioma, malignant schwannoma, osteosarcoma, or soft tissue sarcoma.

The mesothelin-expressing cancer can be a metastatic cancer, e.g., a metastatic solid tumor or a metastatic hematopoietic cancer. The metastatic mesothelin-expressing cancer can be a metastatic ovarian cancer, metastatic mesothelioma, metastatic gastric cancer, or a metastatic lung cancer (e.g., metastatic NSCLC).

The mesothelin-expressing cancer can be a relapsed or refractory cancer, e.g., a relapsed or refractory solid tumor, or a relapsed or refractory hematopoietic cancer. The relapsed or refractory mesothelin-expressing cancer can be a relapsed or refractory ovarian cancer, relapsed or refractory mesothelioma, relapsed or refractory gastric cancer, or a relapsed or refractory lung cancer (e.g., relapsed or refractory NSCLC).

In some embodiments, the method further comprises administering to the subject an additional therapeutic agent or an additional therapeutic regimen. The additional therapeutic agent can comprise a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxaliplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some embodiments, the additional therapeutic agent comprises a first-line therapy. As used herein, “first-line therapy” comprises a primary treatment for a subject with a cancer. In some embodiments, the cancer is a primary cancer. In other embodiments, the cancer is a metastatic or recurrent cancer. In some embodiments, the first-line therapy comprises chemotherapy. In other embodiments, the first-line treatment comprises radiation therapy. A skilled artisan would readily understand that different first-line treatments may be applicable to different type of cancers.

In some embodiments, the additional therapeutic agent comprises an inhibitor of the enzyme poly ADP ribose polymerase (PARP). Exemplary PARP inhibitors include, but are not limited to, olaparib (AZD-2281, Lynparza®, from Astra Zeneca), rucaparib (PF-01367338, Rubraca®, from Clovis Oncology), niraparib (MK-4827, Zejula®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some embodiments, the additional therapeutic agent comprises an immune checkpoint inhibitor. In some embodiments, the checkpoint inhibitor comprises pembrolizumab, nivolumab, tremelimumab, or ipilimumab. In some embodiments, the checkpoint inhibitor comprises an inhibitor of PD-L1, PD-L2, PD-1, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. The inhibitor can be an antibody or fragments (e.g., a monoclonal antibody, a human, humanized, or chimeric antibody) thereof, a RNAi molecule, or a small molecule.

In some embodiments, the additional therapeutic agent comprises an antibody such as alemtuzumab, trastuzumab, ibritumomab tiuxetan, brentuximab vedotin, ado-trastuzumab emtansine, or blinatumomab.

In some embodiments, the additional therapeutic agent comprises a cytokine. Exemplary cytokines include, but are not limited to, IL-1B, IL-6, IL-7, IL-10, IL-12, IL-21, or TNFα.

In some embodiments, the additional therapeutic agent comprises a receptor agonist. In some embodiments, the receptor agonist comprises a Toll-like receptor (TLR) ligand. In some embodiments, the TLR ligand comprises TLR1, TLR2, TLR3, TLR4, TLR5, TLR6, TLR7, TLR8, or TLR9. In some embodiments, the TLR ligand comprises a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some embodiments, the additional therapeutic agent comprises fludarabine and cyclophosphamide.

In some embodiments, the additional therapeutic agent comprises tisagenlecleucel (KYMRIAH®), axicabtagene ciloleucel (YESCARTA®), or brexucabtagene autoleucel (TECARTUS®).

In some embodiments, the additional therapeutic regimen comprises surgery.

In some embodiments, the immune cell described herein or the pharmaceutical composition described herein and the additional therapeutic agent are administered simultaneously.

In some embodiments, the immune cell described herein or the pharmaceutical composition described herein and the additional therapeutic agent are administered sequentially. In some embodiments, the immune cell described herein or the pharmaceutical composition described herein is administered to the subject prior to administration of the additional therapeutic agent. In other embodiments, the immune cell described herein or the pharmaceutical composition described herein is administered to the subject after administration of the additional therapeutic agent.

In some embodiments, the subject is a human.

In some embodiments, also described herein is a method for producing an immune cell expressing cell surface molecules that specifically recognizes mesothelin (e.g., human mesothelin), IL-15, and optionally CCL19. The method comprises introducing a nucleic acid molecule described herein or the vector comprising the nucleic acid molecule to an immune cell to induce expression of cell surface molecules that specifically recognize human mesothelin, IL-15, and optionally CCL19 by the immune cell. In some embodiments, the immune cell is a T cell, a natural killer (NK) cell, a B cell, an antigen presenting cell, or a granulocyte, optionally a T cell or an NK cell.

### Pharmaceutical Composition

In certain embodiments, the immune cells described above are formulated as a pharmaceutical composition. In some embodiments, the pharmaceutical composition is administered to a subject by multiple administration routes, including but not limited to, parenteral, oral, sublingual, or transdermal administration routes. In some embodiments, parenteral administration comprises intravenous, subcutaneous, intramuscular, intranasal, intra-arterial, intra-articular, intradermal, intraosseous infusion, intraperitoneal, subarachnoidal, intracranial, intrasynovial, intratumoral, intracutaneous, intramedullary, intracardiac, or intratechal administration. In some embodiments, the pharmaceutical composition is formulated for local administration. In other embodiments, the pharmaceutical composition is formulated for systemic administration.

In some embodiments, the pharmaceutical composition comprises a pharmaceutically acceptable additive. Examples of the additive can include saline, buffered saline, a cell culture medium, dextrose, injectable water, glycerol, ethanol, a stabilizer, a solubilizer, a surfactant, a buffer, an antiseptic, a tonicity agent, a filler, a lubricant, or a combination thereof.

In some embodiments, the pharmaceutical composition further comprises pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some embodiments, the pharmaceutical composition includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In an exemplary method, the pharmaceutical composition of the present invention can be independently administered in one portion or several divided portions 4 times, 3 times, twice, or once a day, at a 1-day, 2-day, 3-day, 4-day, or 5-day interval, once a week, at a 7-day, 8-day, or 9-day interval, twice a week, once a month, twice a month, three times per month, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In some embodiments, the amount of a given CAR-T cell that correspond to such an amount varies depending upon factors such as the severity of the disease, and the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some embodiments, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Articles of Manufacture

In certain embodiments, disclosed herein is a kit comprising a nucleic acid molecule described above, a vector comprising the nucleic acid molecule described above, an immune cell expressing a CAR that specifically recognizes mesothelin (e.g., human mesothelin), IL-15, and optionally CCL19, or a pharmaceutical composition. In some embodiments, the kit may contain one or more packing materials such as a package insert, a label, a package, or the like stating a use method, etc. for use in the treatment of cancer. Since the immune cell in the pharmaceutical composition of the present invention has suppressive effects on tumor recurrence, the pharmaceutical composition of the present invention may serve as a pharmaceutical composition for use in the suppression of tumor recurrence. Such a pharmaceutical composition for use in the suppression of tumor recurrence may contain one or more packing materials such as a package insert, a label, a package, or the like stating a use method, etc. for use in the suppression of tumor recurrence.

The term “packing material” refers to a physical structure housing a component of the kit. The material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., floppy diskette, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein (e.g., the binding agent or pharmaceutical composition), dose amounts, clinical pharmacology of the active agent(s) including mechanism of action, pharmacokinetics and pharmacodynamics. Labels or inserts can include information identifying manufacturer information, lot numbers, and location and date of manufacture.

Labels or inserts can include information on a disease for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or therapeutic regimes described herein.

Labels or inserts can include information on any benefit that a component may provide, such as a therapeutic benefit. Labels or inserts can include information on potential adverse side effects, such as warnings to the subject or clinician regarding situations where it would not be appropriate to use a particular composition (e.g., a modified immune cell described herein). For example, adverse side effects are generally more likely to occur at higher dose amounts, frequency or duration of the active agent and, therefore, instructions could include recommendations against higher dose amounts, frequency or duration. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−)15%, 10%, 5%, 3%, 2%, or 1%.

Also as used herein, “and/or” refers to and encompasses any and all possible combinations of one or more of the associated listed items, as well as the lack of combinations when interpreted in the alternative (“or”).

As used herein, “optional” or “optionally” means that the subsequently described event or circumstance can or cannot occur, and that the description includes embodiments where the event or circumstance occurs and embodiments where it does not.

As used herein, the term “antibody” refers to a protein that binds to other molecules (antigens, e.g., mesothelin) via heavy and light chain variable domains, V_{H} and V_{L}, respectively. The term “variable region” or “variable domain” refers to the domain of an antibody that is involved in binding the antibody to antigen. The variable domains of the heavy chain and light chain (VH and VL, respectively) of a native antibody generally have similar structures, with each domain comprising four conserved framework regions (FRs) and three CDRs. (See, e.g., Kindt et al. Kuby Immunology, 6^{th} ed., W. H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Antibodies of the disclosure include monoclonal antibodies. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Monoclonal antibodies are made by methods known in the art (Kohler et al., Nature, 256:495 (1975); and Harlow and Lane, Using Antibodies: A Laboratory Manual, Cold Spring Harbor Laboratory, 1999). Briefly, monoclonal antibodies can be obtained by injecting mice with antigen. The polypeptide or peptide used to immunize an animal may be derived from translated DNA or chemically synthesized and conjugated to a carrier protein. Commonly used carriers which are chemically coupled to the immunizing peptide include, for example, keyhole limpet hemocyanin (KLH), thyroglobulin, bovine serum albumin (BSA), and tetanus toxoid. Antibody production is verified by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from hybridoma cultures. Monoclonal antibodies can be isolated and purified from hybridoma cultures by a variety of established techniques which include, for example, affinity chromatography with Protein-A Sepharose, size-exclusion chromatography, and ion-exchange chromatography (see e.g., Coligan et al., Current Protocols in Immunology sections 2.7.1-2.7.12 and sections 2.9.1-2.9.3; and Barnes et al., “Methods in Molecular Biology,” 10:79-104, Humana Press (1992)).

Antibodies of the disclosure can belong to any antibody class, IgM, IgG, IgE, IgA, IgD, or subclass. Exemplary subclasses for IgG are IgG₁, IgG₂, IgG₃ and IgG₄.

Antibodies of the disclosure can be a humanized antibody. The term “humanized” refers to an antibody sequence that has non-human amino acid residues of one or more complementarity determining regions (CDRs) that specifically bind to the antigen in an acceptor human immunoglobulin molecule, and one or more human amino acid residues in the framework region (FR) that flank the CDRs. Any mouse, rat, guinea pig, goat, non-human primate (e.g., ape, chimpanzee, macaque, orangutan, etc.) or other animal antibody may be used as a CDR donor for producing humanized antibody. Human framework region residues can be replaced with corresponding non-human residues (e.g., from the donor variable region). Residues in the human framework regions can therefore be substituted with a corresponding residue from the non-human CDR donor antibody. A humanized antibody may include residues, which are found neither in the human antibody nor in the donor CDR or framework sequences. The use of antibody components derived from humanized monoclonal antibodies reduces problems associated with the immunogenicity of non-human regions. Methods of producing humanized antibodies are known in the art (see, for example, U.S. Pat. Nos. 5,225,539; 5,530,101, 5,565,332 and 5,585,089; Riechmann et al., (1988) Nature 332:323; EP 239,400; WO91/09967; EP 592,106; EP 519,596; Padlan Molecular Immunol. (1991)28:489; Studnicka et al., Protein Engineering (1994)7:805; Singer et al., J. Immunol. (1993)150:2844; and Roguska et al., Proc. Nat'l. Acad. Sci. USA (1994)91:969).

Antibodies of the disclosure can be a chimeric antibody. The term “chimeric antibody” refers to an antibody in which different portions are derived from different animal species, such as those having a variable region derived from a murine monoclonal antibody and a human immunoglobulin constant region, e.g., humanized antibodies. In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used.

Antibodies of the disclosure include binding fragments thereof. Exemplary antibody fragments include Fab, Fab′, F(ab′)₂, Fv, Fd, single-chain Fv (scFv), disulfide-linked Fvs (sdFv), light chain variable region V_{L}, heavy chain variable region VH, trispecific (Fab₃), bispecific (Fab₂), diabody ((V_{L}-V_{H})₂ or (V_{H}-V_{L})₂), triabody (trivalent), tetrabody (tetravalent), minibody ((scFv-CH)₂), bispecific single-chain Fv (Bis-scFv), IgGdeltaCH2, scFv-Fc, (scFv)₂-Fc and IgG4PE. Such fragments can have the binding affinity as the full length antibody, the binding specificity as the full length antibody, or one or more activities or functions of as a full length antibody, e.g., a function or activity of mesothelin binding antibody.

Antibody fragments can be combined. For example, a V_{L} or V_{H} subsequences can be joined by a linker sequence thereby forming a V_{L}-VI chimera. A combination of single-chain Fvs (scFv) sequences can be joined by a linker sequence thereby forming a scFv-scFv chimera. Antibody fragments include single-chain antibodies or variable region(s) alone or in combination with all or a portion of other sequences.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

Antibody fragments can also be prepared by proteolytic hydrolysis of the antibody, for example, by pepsin or papain digestion of whole antibodies. Antibody fragments produced by enzymatic cleavage with pepsin provide a 5S fragment denoted F(ab′)₂. This fragment can be further cleaved using a thiol reducing agent to produce 3.5S Fab′ monovalent fragments. Alternatively, an enzymatic cleavage using pepsin produces two monovalent Fab′ fragments and the Fc fragment directly (see, e.g., U.S. Pat. Nos. 4,036,945 and 4,331,647; and Edelman et al., Methods Enymol. 1:422 (1967)). Other methods of cleaving antibodies, such as separation of heavy chains to form monovalent light-heavy chain fragments, further cleavage of fragments, or other enzymatic or chemical may also be used.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

As used herein, “identical”, “sequence identity”, or percent “identity”, when used in the context of two or more nucleic acids or polypeptide sequences, refers to two or more sequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region. The alignment and sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “identical”, “sequence identity”, or percent “identity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The term “protein”, “peptide”, and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. Polypeptides include full length native polypeptide, and “modified” forms such as subsequences, variant sequences, fusion/chimeric sequences and dominant-negative sequences. As used herein the term “amino acid” refers to either natural and/or unnatural or synthetic amino acids, including glycine and both the D and L optical isomers, amino acid analogs and peptidomimetics.

Peptides include L- and D-isomers, and combinations thereof. Peptides can include modifications typically associated with post-translational processing of proteins, for example, cyclization (e.g., disulfide or amide bond), phosphorylation, glycosylation, carboxylation, ubiquitination, myristylation, or lipidation. Modified peptides can have one or more amino acid residues substituted with another residue, added to the sequence or deleted from the sequence. Specific examples include one or more amino acid substitutions, additions or deletions (e.g., 1-3, 3-5, 5-10, 10-20, or more).

As used herein, the terms “modification” and “modified” refer to a mutation, substitution, addition, or deletion of one or more amino acid residues of an antibody, protein, or polypeptide in comparison to a reference antibody, protein, or polypeptide that is the equivalent of the antibody, protein, or polypeptide without the modification. In some embodiments, the modification comprises a conservative substitution.

A “conservative substitution” is the replacement of one amino acid by a biologically, chemically or structurally similar residue. Biologically similar means that the substitution is compatible with an activity or function of the unsubstituted sequence. Structurally similar means that the amino acids have side chains with similar length, such as alanine, glycine and serine, or having similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

As used herein, the term “nucleic acid” refers to a DNA or an RNA, comprising natural, synthetic, or artificial nucleotide analogues or bases. In some embodiments, a nucleotide analogue or artificial nucleotide base comprises a nucleic acid with a modification at a 2′ hydroxyl group of the ribose moiety. In some embodiments, the modification includes an H, OR, R, halo, SH, SR, NH₂, NHR, NR₂, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some embodiments, the alkyl moiety further comprises a modification. In some embodiments, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some embodiments, the alkyl moiety further comprises a hetero substitution. In some embodiments, the carbon of the heterocyclic group is substituted by a nitrogen, oxygen or sulfur. In some embodiments, the heterocyclic substitution includes but is not limited to, morpholino, imidazole, and pyrrolidino.

In some embodiments, a nucleotide analogue comprises a modified base such as, but not limited to, 5-propynyluridine, 5-propynylcytidine, 6-methyladenine, 6-methylguanine, N, N,-dimethyladenine, 2-propyladenine, 2propylguanine, 2-aminoadenine, 1-methyl inosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some embodiments are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

The nucleic acid molecules of the present invention can be produced by a publicly known technique such as a chemical synthesis method or a PCR amplification method on the basis of information on the nucleotide sequence of each of the nucleic acids. Codons selected for encoding amino acids may be engineered in order to optimize nucleic acid expression in host cells of interest.

In the present invention, the “expression of IL-15” by T cells means that various forms of IL-15 (e.g., wild-type, variants, or fusion proteins described above) can be expressed so long as the effects of the present invention are not lost.

As used herein, the term “substantially” when describing the population of T cells refers to a population comprising less than about 30%, 25%, 20%, 15%, 10%, 5%, or less contaminant cells. In some embodiments, the contaminant cells are less than about 20% in the population of T cells. In some embodiments, the contaminant cells are less than about 15% in the population of T cells. In some embodiments, the contaminant cells are less than about 10% in the population of T cells.

As used herein, the terms “treating,” “treatment” and the like mean obtaining a desired pharmacologic and/or physiologic effect. The effect may be therapeutic in terms of amelioration of the symptoms of the disease, or a partial or complete cure for a disease and/or adverse effect attributable to the disease. In one aspect, the term “treatment” excludes prophylaxis.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disease, such as one or more adverse symptoms, disorders, illnesses, pathologies, diseases, or complications caused by or associated with the disease, or an improvement in an underlying cause or a consequence of the disease, or a reversal of the disease.

Treatment can therefore result in decreasing, reducing, inhibiting, suppressing, limiting, controlling or preventing a disease, or an associated symptom or consequence, or underlying cause; decreasing, reducing, inhibiting, suppressing, limiting, controlling or preventing a progression or worsening of a disease, condition, symptom or consequence, or underlying cause; or further deterioration or occurrence of one or more additional symptoms of the disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disease or symptom in the subject, such as one or more adverse symptoms, disorders, illnesses, pathologies, diseases, or complications caused by or associated with a disease or condition. Treatment methods affecting one or more underlying causes of the condition, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the disease or condition, over a short or long duration of time (hours, days, weeks, months, etc.).

The terms “acceptable,” “effective,” or “sufficient” when used to describe the selection of any components, ranges, dose forms, etc. disclosed herein intend that said component, range, dose form, etc. is suitable for the disclosed purpose.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human.

As used herein, the term “γδT cell” means a cell that expresses CD3, and expresses TCR constituted of TCRγ chain (γTCR) and TCRδ chain (δTCR) (hereinafter sometimes to be referred to as “γδTCR”).

As used herein, the term “αβT cell” means a cell that expresses CD3, and expresses TCR constituted of TCRα chain (αTCR) and TCRβ chain (βTCR) (hereinafter sometimes to be referred to as “αβTCR”). In some instances, almost all αβT cells recognize antigen peptide-MHC (major histocompatibility complex, in the case of human, HLA: human leukocyte antigen) complex by αβTCR (this is to be referred to as MHC restriction). In contrast, γδT cell recognizes various molecules expressed by cells, by γδTCR regardless of MHC molecule.

As used herein, the term “modulate” or “modulator” refers to the ability of a component to positively or negatively alter an associated function. Exemplary modulations include about 1%, about 2%, about 5%, about 10%, about 25%, about 50%, about 75%, or about 100% change.

In certain embodiments, provided herein are TGFβ signaling modulators capable of altering or preventing TGFβ receptor from signaling. Those skilled in the art would understand that this can be achieved by either binding the cytokine (i.e., TGFβ) which activates the signaling of TGFβR, or the receptor itself (e.g., a TGFβ antibody or fragment thereof, a TGFβR antibody or fragment thereof). Therefore this term encompasses both molecules which bind TGFβ and molecules which bind TGFβR. In one embodiment, the modulator of the disclosure can neutralize TGFβ signaling through TGFβRII. By “neutralizing”, it is meant that the normal signaling effect of TGFβ is blocked such that the presence of TGFβ has a neutral effect on TGFβRII signaling. In some embodiments, TGFβ modulators improve the immune response in a host.

As used herein, the terms “transforming growth factor-β”, “TGF-beta”, “TGFb”, “TGFβ” and “transforming growth factor-beta” are used interchangeably herein and refer to the family of molecules that have either the full-length, native amino acid sequence of any of the TGF-betas from humans, including the latent forms and associated or unassociated complex of precursor and mature TGF-beta (“latent TGF-beta”). Reference to such TGF-beta herein will be understood to be a reference to any one of the currently identified forms, including TGF-beta1, TGF-beta2, TGF-beta3, TGF-beta4, and TGF-beta5 and latent versions thereof, as well as to human TGF-beta species identified in the future, including polypeptides derived from the sequence of any known TGF-beta and being at least about 75%, preferably at least about 80%, more preferably at least about 85%, still more preferably at least about 90%, and even more preferably at least about 95% homologous with the sequence. The specific terms “TGF-beta1,” “TGF-beta2,” and “TGF-beta3”, as well as “TGF-beta4” and “TGF-beta5,” refer to the TGF-betas defined in the literature, e.g., Derynck et al., Nature, supra, Seyedin et al., J. Biol. Chem., 262, supra, and deMartin et al., supra. The term “TGF-beta” refers to the gene encoding human TGF-beta. The preferred TGF-beta is native-sequence human TGF-beta.

Members of the TGF-beta family are defined as those that have nine cysteine residues in the mature portion of the molecule, share at least 65% homology with other known TGF-beta sequences in the mature region, and may compete for the same receptor. In addition, they all appear to be encoded as a larger precursor that shares a region of high homology near the N-terminus and shows conservation of three cysteine residues in the portion of the precursor that will later be removed by processing. Moreover, the TGF-betas appear to have a processing site with four or five amino acids.

As used herein, the terms “transforming growth factor-β receptor”, “TGF-bR” or “TGF-b receptor” or “TGF-beta receptor” or “TGFβR” is used to encompass all three sub-types of the TGFβR family (i.e., TGFβR1, TGFβR2, TGFβR3). The TGFβ receptors are characterized by serine/threonine kinase activity and exist in several different isoforms that can be homo- or heterodimeric.

As used herein, the term “TGFβ signaling pathway modulator”, “TGFβ modulator”, “TGFβ signaling pathway modulator” or “TGFβ modulator”, as used interchangeably herein, refers to a molecule (e.g., an antibody or fragment thereof) which is capable of modulating TGFβ signaling pathway (e.g., having an inhibiting, blocking or neutralizing effect), which may either bind TGFβ itself or it may bind a TGFβ receptor on cells. In either case, the modulator inhibits the TGFβ signaling pathway (e.g., by either binding the cytokine (i.e., TGFβ) itself) or by binding the receptor for TGFβ. Therefore this term encompasses both types of modulators, which bind TGFβ and those which bind the TGFβ receptor. In various embodiments described herein a TGFβ signaling pathway modulator is expressed along with a chimeric antigen receptor in a modified immune cell (e.g., a CAR-T cell). In various embodiments described herein a TGFβ signaling pathway modulator is secreted from a modified immune cell or expressed as bounded on the membrane of an immune cell. CAR-T cells expressing such a TGFβ signaling pathway modulator are referred to herein as TGFβ armored CAR-T cells

As used here, the term “induced pluripotent stem cell” (hereinafter sometimes to be referred to as “iPS cell”) means a stem cell that is established by introducing a reprogramming factor into a somatic cell, has pluripotency permitting differentiation into many cells present in living organisms, and also has proliferation capacity. It encompasses any cell induced into a hematopoietic progenitor cell to be used in the present invention. The induced pluripotent stem cell is preferably derived from a mammal (e.g., mouse, rat, hamster, guinea pig, dog, monkey, orangutan, chimpanzee, human), more preferably human.

In some instances, a pluripotent stem cell can be induced or established by introducing a reprogramming factor into any somatic cell. Exemplary reprogramming factors include, but not limited to, genes and gene products such as Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sall1, Sall4, Esrrb, Nr5a2, Tbx3, Glis1 and the like. These reprogramming factors may be used alone or in combination. In some instances, the combination reprogramming factor is exemplified by the combinations described in WO 2007/069666, WO 2008/118820, WO 2009/007852, WO 2009/032194, WO 2009/058413, WO 2009/057831, WO 2009/075119, WO 2009/079007, WO 2009/091659, WO 2009/101084, WO 2009/101407, WO 2009/102983, WO 2009/114949, WO 2009/117439, WO 2009/126250, WO 2009/126251, WO 2009/126655, WO 2009/157593, WO 2010/009015, WO 2010/033906, WO 2010/033920, WO 2010/042800, WO 2010/050626, WO 2010/056831, WO 2010/068955, WO 2010/098419, WO 2010/102267, WO 2010/111409, WO 2010/111422, WO 2010/115050, WO 2010/124290, WO 2010/147395, WO 2010/147612, Huangfu D, et al. (2008), Nat. Biotechnol., 26:795-797, Shi Y, et al. (2008), Cell Stem Cell, 2:525-528, Eminli S, et al. (2008), Stem Cells. 26:2467-2474, Huangfu D, et al. (2008), Nat. Biotechnol. 26:1269-1275, Shi Y, et al. (2008), Cell Stem Cell, 3, 568-574, Zhao Y, et al. (2008), Cell Stem Cell, 3:475-479, Marson A, (2008), Cell Stem Cell, 3, 132-135, Feng B, et al. (2009), Nat. Cell Biol. 11:197-203, R. L. Judson et al., (2009), Nat. Biotechnol., 27:459-461, Lyssiotis CA, et al. (2009), Proc Natl Acad Sci USA. 106:8912-8917, Kim J B, et al. (2009), Nature. 461:649-643, Ichida J K, et al. (2009), Cell Stem Cell. 5:491-503, Heng J C, et al. (2010), Cell Stem Cell. 6:167-74, Han J, et al. (2010), Nature. 463:1096-100, Mali P, et al. (2010), Stem Cells. 28:713-720, and Maekawa M, et al. (2011), Nature. 474:225-9.

Examples of the somatic cells include, but are not limited to, any of fetal somatic cells, neonatal somatic cells, and mature somatic cells, as well as any of primary cultured cells, subcultured cells, and established cell lines. Furthermore, the cells described above may be healthy cells or diseased cells. Specific examples of the somatic cells include (1) tissue stem cells (somatic stem cells) such as neural stem cells, hematopoietic progenitor cells, mesenchymal stem cells, and dental pulp stem cells; (2) tissue progenitor cells; and (3) differentiated cells such as blood cells (e.g., peripheral blood cells, cord blood cells, and the like), mononuclear cell (e.g., lymphocyte (NK cells, B cells, T cells other than αβT cells (e.g., γδT cells and the like), monocyte, dendritic cell and the like)), granulocyte (e.g., eosinophils, neutrophil, basophil), megakaryocyte), epithelial cells, endothelial cells, muscle cells, fibroblasts (e.g., skin cells and the like), hair cells, hepatic cells, gastric mucosal cells, enterocytes, spleen cells, pancreatic cells (e.g., pancreatic exocrine cells and the like), brain cells, lung cells, kidney cells, and adipocytes. Among these, a mononuclear cell other than an αβT cell is preferable, more specifically, a monocyte or γδT cell is preferable.

Exemplary methods such as calcium phosphate coprecipitation method, PEG method, electroporation method, microinjection method, lipofection method and the like can be used to introduce a reprogramming factor into a somatic cell. Additional methods can include those described in Cell Engineering additional volume 8, New Cell Engineering experiment protocol, 263-267 (1995) (published by Shujunsha), Virology, vol. 52, 456 (1973), and Folia Pharmacol. Jpn., vol. 119 (No. 6), 345-351 (2002). When a virus vector is used, the nucleic acid is introduced into a suitable packaging cell (e.g., Plat-E cell) and complementation cell line (e.g., 293 cell), a virus vector produced in the culture supernatant is recovered, and cells are infected with the vector by an appropriate method suitable for each virus vector, whereby the vector is introduced into the cells. For example, when a retrovirus vector is used as the vector, a specific means is disclosed in WO 2007/69666, Cell, 126, 663-676 (2006) and Cell, 131, 861-872 (2007) and the like. Particularly, when a retrovirus vector is used, highly efficient transfection into various cells is possible by using a recombinant fibronectin fragment CH-296 (manufactured by Takara Bio Inc.).

A reprogramming factor in the form of RNA may be directly introduced into cells and expressed in the cells. As a method for introducing RNA, a known method can be used and, for example, a lipofection method, an electroporation method, or the like can be preferably used. When the reprogramming factor is in the form of a protein, it can be introduced into a cell by a method such as lipofection, fusion with cellular membrane-penetrating peptide (e.g., HIV-derived TAT and polyarginine), microinjection and the like.

In some embodiments, a method for differentiating induced pluripotent stem cells into T cells is not particularly limited as long as induced pluripotent stem cells can be differentiated into γδT cells. In some instances, the method comprises differentiating induced pluripotent stem cells into hematopoietic progenitor cells and differentiating the hematopoietic progenitor cells into CD3 positive T cells. Exemplary methods for use in differentiating iPSCs into hematopoietic progenitor cells include those disclosed in WO 2013/075222, WO 2016/076415 and Liu S. et al., Cytotherapy, 17 (2015); 344-358. Exemplary methods for use in differentiating the hematopoietic progenitor cells into CD3 positive T cells include those disclosed in WO 2016/076415, and WO 2017/221975. Also see the methods disclosed in WO2020/013315.

### EXAMPLES

These examples are provided for illustrative purposes only and not to limit the scope of the claims provided herein.

### Example 1

### Production Method of Cells Expressing γδTCR

A suspended cell population differentiated from iPS cells (Ff-101s04 strain: derived from peripheral blood mononuclear cell of a healthy individual) provided by the Center for iPS Cell Research and Application, Kyoto University, using a known culturing method (e.g., as described in Cell Reports 2 (2012)₁₇₂₂-1735 or WO 2017/221975) was used to generate a cell population containing hematopoietic progenitor cells (HPCs). For example, Ff-101s04 strain was seeded at 6×10⁵ cells/well (Day 0) in an ultra-low adhesion-treated 6 well plate, 10 ng/ml BMP4, 50 ng/ml bFGF, 15 ng/ml VEGF, 2 μM SB431542 were added to EB medium (StemPro34 added with 10 μg/ml human insulin, 5.5 g/ml human transferrin, 5 ng/ml sodium selenite, 2 mM L-glutamine, 45 mM α-monothioglycerol, and 50 μg/ml Ascorbic acid 2-phosphate), and the cells were cultured for 5 days under low-oxygen conditions (5% O2) (Day 5). Then, 50 ng/ml SCF, 30 ng/ml TPO, 10 ng/ml FLT-3L were added, and the cells were cultured for 5-9 days (-Day 14) to yield a suspended cell population. The medium was changed every two or three days during the culture period. The resultant suspended cell population containing HPC was stained using the antibody set as shown in Table 2.

TABLE 2

| | |
|---|---|
| anti-CD34 antibody | Abcam PE/Cy7 |
| anti-CD43 antibody | BD APC |
| anti-CD45 antibody | BioLegend BV510 |
| anti-CD14 antibody | BioLegend APC/eFluor780 |
| anti-CD235a antibody | BD FITC |

The cell populations that underwent the above-mentioned staining were subjected to sorting by FACSAria. The obtained cell fractions were differentiated into lymphoid cells according to a known method (e.g., the methods described in Journal of Leukocyte Biology 96 (2016)1165-1175 and WO 2017/221975). For example, the hematopoietic progenitor cell population was seeded at 2000 cells/well in a 48-well-plate coated with Recombinant h-DLL4/Fc chimera (Sino Biological) and Retronectin (Takara Bio Inc.) and cultured under 5% CO₂, 37° C. conditions. The medium was changed every two or three days during the culture period. The αMEM medium contained 15% FBS, 2 mM L-glutamine, 100 U/ml penicillin, 100 ng/ml streptomycin, 55μμ 2-mercaptoethanol, 50 μg/ml Ascorbic acid 2-phosphate, 10 μg/ml human insulin, 5.5 μg/ml human transferrin, 5 ng/ml sodium selenite, 50 ng/ml SCF, 50 ng/ml IL-7, 50 ng/ml FLT-3L, 100 ng/ml TPO, 15 μM SB203580, and 30 ng/ml SDF-1α. The cells were passaged to a similarly-coated 48-well plate on day 7 and day 14 from the start of the culture. All cells were collected on day 21 from the start of the culture (Day 35) and the presence of CD45 (+), CD3 (+) fractions was confirmed by a flow cytometer (BD FACSAria™ Fusion, manufactured by BD Biosciences). The obtained cells were seeded in a 48-well plate and cultured under 5% CO₂, 37° C. conditions. The αMEM medium contained 15% FBS, 2 mu L-glutamine, 100 U/ml penicillin, 100 ng/ml streptomycin, 50 μg/ml Ascorbic acid 2-phosphate, 10 μg/ml human insulin, 5.5 μg/ml human transferrin, 5 ng/mL sodium selenite, 500 ng/mL anti-CD3 antibody (OKT3), and 10 ng/ml IL-7. All cells were collected on day 27 from the start of the culture (Day 41), the cells were counted by a hemocytometer, and stained using the antibody set as shown in Table 3.

TABLE 3
Vδ1 Miltenyi FITC
Vδ2 Miltenyi APC
γδTCR BD BV510
CD3 BioLegend APC/Cy7
αβTCR eBioscience FITC

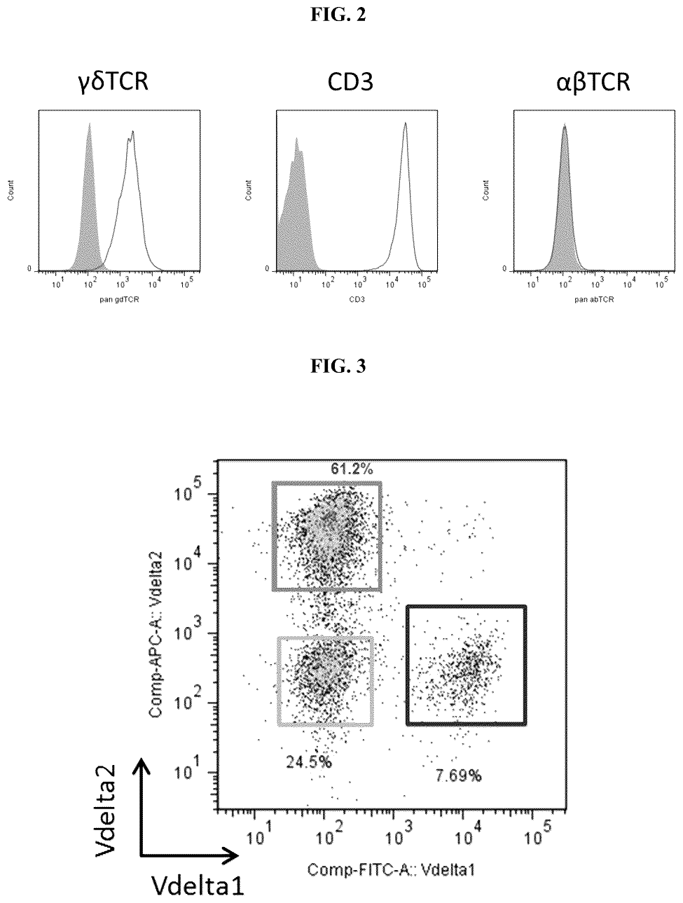

As a result of the staining, it was shown that a cell expressing γδTCR (γδTCR positive cell) can be prepared from a hematopoietic progenitor cell derived from an iPS cell (Ff-I01s04 strain) (FIG. 2).

Furthermore, since the γδTCR positive cell contains Vδ1 positive γδT cell and Vδ2 positive γδT cell, it was shown that Vδ1 type and Vδ2 type γδT cells can be prepared (FIG. 3). (Note the iPS cell-derived γδTCR positive cell without KI of γδTCR gene are also referred to herein as “iγδT cells”)

### Example 2

Expansion Culture and Function Evaluation of iγδT Cells

Production of iγδT cells was carried out using the same method as in Example 1 except that UCHT1 (manufactured by GeneTex) was used as the anti-CD3 antibody, γδT cells (iγδT cells) derived from iPS cells (Ff-101s04 strain) were produced.

The subsequent iγδT cells obtained were suspended at 2,000,000 cells/mL in an α-MEM medium containing 15% FBS and an additive containing cytokine as shown in Table 4, seeded on a plate solid-phased with anti-CD3 antibody (UCHT1) and RetroNectin, and cultured at 5% CO₂/37° C. for 3 days. On the 3rd day of culture, the cells were collected from the plate, the number of cells was counted using NucleoCounter (registered trade mark) NC-200 (ChemoMetec), and the cells were suspended in an appropriate amount in an α-MEM medium containing 15% FBS and an additive containing cytokine as shown in Table 5, added to a non-immobilized G-Rex (registered trade mark) 6-well plate (WILSONWOLF), and cultured at 5% CO₂/37° C. A part of the cells were collected from the plate 4-6 times on days 5, 6, 7, 8, 9, 10, 11, 14, and 17, and the number of the cells was counted using a hemocytometer.

The anti-CD3 antibody and RetroNectin were immobilized on the culture plate by the following method. The anti-CD3 antibody (UCHT1 final concentration 3000 ng/mL) and RetroNectin (final concentration 150 μg/mL) dissolved in PBS at necessary concentrations were added to the plate and then allowed to stand overnight at 4° C. After washing with PBS, the plate was subjected to the test.

TABLE 4

| product name | manufacturer | Final conc | |
|---|---|---|---|
| Insulin-Transferrin-Selenium | Invitrogen | 1× | |
| Supplements | | | |
| Ascorbic acid 2-phosphate | sigma | 50 | μg/ml |
| IL-2 | Peprotech | 15 | ng/ml |
| IL-7 | Peprotech | 10 | ng/ml |
| IL-15 | Peprotech | 10 | ng/ml |
| IL-21 | Peprotech | 20 | ng/ml |
| IL-12 | Merck | 50 | ng/ml |
| IL-18 | MBL | 50 | ng/ml |
| TL-1A | Peprotech | 50 | ng/ml |
| Z-VAD-FMK | R&D | 10 | μM |
| Human CD30 Antibody | R&D | 300 | ng/ml |

TABLE 5

| product name | manufacturer | Final conc | |
|---|---|---|---|
| Insulin-Transferrin-Selenium | Invitrogen | 1× | |
| Supplements | | | |
| Ascorbic acid 2-phosphate | sigma | 50 | μg/ml |
| IL-2 | Peprotech | 15 | ng/ml |
| IL-7 | Peprotech | 10 | ng/ml |
| IL-15 | Peprotech | 10 | ng/ml |
| Human CD30 Antibody | R&D | 300 | ng/ml |

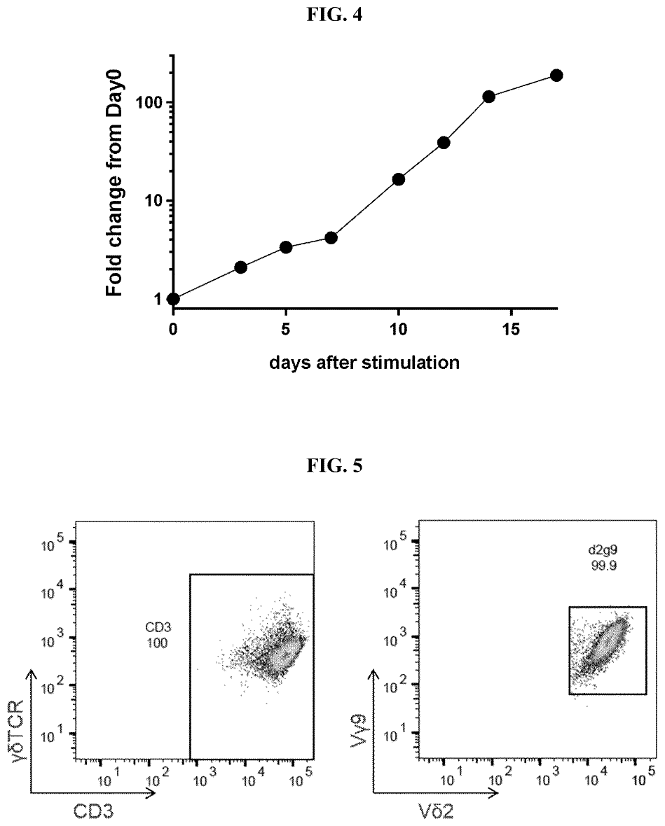

Proliferation of iγδT cells was observed by stimulation with anti-CD3 antibody (UCHT1) and anti-CD30 antibody (FIG. 4).

### Example 3

### Production of iPS Cell-Derived Vγ9Vδ2T Cells

### 1. Preparation of iPS Cell

The iPS cells were prepared similarly as disclosed in Example 1. In particular, Ff-101s04 strain provided by the Center for iPS Cell Research and Application (CiRA), Kyoto University, was used. iPS cells were cultured according to the protocol “feeder-free culture of human iPS cells” distributed by CiRA.

2. Differentiation of iPS Cell into HPC

Differentiation of iPS cells into hematopoietic progenitor cells (HPC) was performed according to a method disclosed in WO 2017/221975.

### 3. Vγ9Vδ2 Gene

G115γδT cell clone-derived Vγ9Vδ2 T cell receptor (Vγ9Vδ2TCR G115) was used. The nucleic acid including a gene encoding Vγ9Vδ2TCR G115, which encodes SEQ ID NO: 44, contains the genes as shown in Table 6. The nucleic acid was artificially synthesized.

TABLE 6

| order from | | Residue numbering according |
|---|---|---|
| N-terminal | Genes | to SEQ ID NO: 44 |
| 1 | (G115-derived) TRG | 1-315 |
| 2 | P2A | 316-337 |
| 3 | (G115-derived) TRD | 338-629 |

(SEQ ID NO: 44)
MVSLLHASTLAVLGALCVYGAGHLEQPQISSTKTLSKTARLECVVSG
ITISATSVYWYRERPGEVIQFLVSISYDGTVRKESGIPSGKFEVDRIPE
TSTSTLTIHNVEKQDIATYYCALWEAQQELGKKIKVFGPGTKLIITDKQ
LDADVSPKPTIFLPSIAETKLQKAGTYLCLLEKFFPDVIKIHWEEKKSN
TILGSQEGNTMKINDTYMKFSWLTVPEKSLDKEHRCIVRHENNKNGVDQ
EIIFPPIKTDVITMDPKDNCSKDANDTLLLQLTNTSAYYMYLLLLLKSV
VYFAIITCCLLRRTAFCCNGEKSGSGATNFSLLKQAGDVEENPGPMERI
SSLIHLSLFWAGVMSAIELVPEHQTVPVSIGVPATLRCSMKGEAIGNYY
INWYRKTQGNTMTFIYREKDIYGPGFKDNFQGDIDIAKNLAVLKILAPS
ERDEGSYYCACDTLGMGGEYTDKLIFGKGTRVTVEPRSQPHTKPSVFVM
KNGTNVACLVKEFYPKDIRINLVSSKKITEFDPAIVISPSGKYNAVKLG
KYEDSNSVTCSVQHDNKTVHSTDFEVKTDSTDHVKPKETENTKQPSKSC
HKPKAIVHTEKVNMMSLTVLGLRMLFAKTVAVNFLLTAKLFFL.

### 4. Production of Retrovirus Vector Carrying Vγ9Vδ2 Gene

The lentivirus vector pLVSIN-Ub was used and the vector was generated by removing the sequence encoding the neomycin resistance gene from the pLVSIN-CMV Neo vector (Clontech Laboratories, Inc.) and the CMV promoter was replaced with human ubiquitin promoter. The artificial oligo DNA was synthesized according to Table 6. The Vγ9Vδ2 gene was incorporated into the multi-cloning site of the pLVSIN-Ub retrovirus vector. Using this plasmid and the Lenti-X™ 293T cell line and the Lenti-X™ Packaging Single Shots (VSV-G) of Clontech Laboratories, Inc., the lentiviral vector pLVSIN-Ub was produced.

### 5. Production of iPS Cell-Derived Vγ9Vδ2T Cells

The iPS cells prepared in 1. Preparation of iPS cell were infected with the retrovirus vector prepared in 4. Production of retrovirus vector carrying Vγ9Vδ2 gene carrying the Vγ9Vδ2 gene. These cells were differentiated into T cells according to a known method (as disclosed in WO2017/221975) in the same manner as in Example 1 to prepare iPS cell-derived Vγ9Vδ2T cells. 500 ng/mL OKT3 (TakaraBio) was used as the anti-CD3 antibody in the differentiation step. (Note the iPS cell-derived Vγ9Vδ2T cells prepared from iPS cells are also referred to herein as “iγ9δ2T cells”.) The obtained iγ9δ2T cells were measured for the expression of CD3, γδTCR, Vγ9 and Vδ2 on the cell membrane surface with a flow cytometer (BD FACSAria™ Fusion, manufactured by BD Biosciences (FIG. 5).

### Example 4

### Production of iPS Cell-Derived Anti-Mesothelin-CAR/IL-15γδT Cells

### 1, Anti-Mesothelin-CAR Gene

The nucleic acids comprising the gene encoding anti-Mesothelin-CAR were artificially synthesized. 11 patterns of artificial nucleic acids were synthesized for the transfection into iγδT cells or iγ9δ2T cells using the genes listed in Table 7.

TABLE 7

| | order from N- | |
|---|---|---|
| Abbreviation | terminal | gene |
| ss VH | 1 | lead sequence of immunoglobulin heavy |
| | | chain |
| P4 scFv (P4) | 2 | variable region of anti-Mesothelin antibody |
| | | (P4) heavy chain |
| | 3 | GILGSGGGGSGGGGSGGGGS linker |
| | | (SEQ ID NO: 45) |
| | 4 | variable region of anti-Mesothelin antibody |
| | | (P4) light chain |
| CD8hinge/TM | 5 | CD8-derived sequence (including |
| | | transmembrane region) |
| CD28 | 6a (either) | intracellular domain region of CD28 |
| 41BB | 6b (or) | intracellular domain region of 4-1BB |
| CD3z | 7 | intracellular domain region of CD3ζ |
| P2A | 8 | P2A |
| IL2sp or | 9 | Signal peptide |
| IL15Rasp | (with/without) | |
| mIL 15/Ra, | 10 | IL-15 |
| mIL 15/Ra- | | |
| LSP, sushi15 | | |
| or sIL15-LSP | | |
| P2A | 11 | P2A |
| | (with/without) | |
| endosp | 12 | Signal peptide |
| CCL19 | 13 | CCL19 |
| | (with/without) | |

### 2. IL-15 Genes

The artificial nucleic acid comprised the gene encoding the each IL-15 version. The genes of IL-15 versions are shown in Table 8.

TABLE 8

| | order | | |
|---|---|---|---|
| | from | | SEQ |
| | N- | | ID |
| Abbreviation | terminal | Genes | NO: |
| mIL 15/Ra | 1 | IL2sp (SEQ ID NO: 6) | 28 |
| (with IL2sp) | 2 | C-terminal sequence of human IL-15 | |
| | 3 | Peptide linker (SEQ ID NO: 32) | |
| | 4 | C-terminal sequence of human | |
| | | IL-15RA | |
| sushi 15 | 1 | IL 15Rasp (SEQ ID NO: 7) | 29 |
| (with IL 15Rasp) | 2 | sushi 15(SEQ ID NO: 10) | |
| | 3 | Peptide linker (SEQ ID NO: 32) | |
| | 4 | C-terminal sequence of human | |
| | | IL-15RA | |
| mIL 15/Ra-LSP | 1 | IL-15Ra-LSP | 9 |
| | 2 | IL-15 propeptide | |
| | 3 | C-terminal sequence of human IL-15 | |
| | 4 | Peptide linker (SEQ ID NO: 32) | |
| | 5 | C-terminal sequence of human | |
| | | IL-15RA | |
| SIL 15-LSP | 1 | IL-15Ra-LSP | 11 |
| | 2 | IL-15 propeptide | |
| | 3 | C-terminal sequence of human IL-15 | |

The 1^{st} pattern of the artificial nucleic acid comprises ssV_{H}-P4-CD8hinge/TM-41BB-CD3z-P2A-IL2sp-mIL15/Ra in this order from N-terminal.

The 2^{nd} pattern of the artificial nucleic acid comprises ssV_{H}-P4-CD8hinge/TM-41BB-CD3z-P2A-mIL15/Ra-LSP in this order from N-terminal.

The 3^{rd} pattern of the artificial nucleic acid comprises ssV_{H}-P4-CD8hinge/TM-41BB-CD3z-P2A-IL15Rasp-sushi15 in this order from N-terminal.

The 4^{th} pattern of the artificial nucleic acid comprises ssV_{H}-P4-CD8hinge/TM-41BB-CD3z-P2A-sIL15-LSP in this order from N-terminal.

The 5^{th} pattern of the artificial nucleic acid comprises ssV_{H}-P4-CD8hinge/TM-41BB-CD3z-P2A-IL2sp-mIL15/Ra-P2A-endosp-CCL19 in this order from N-terminal.

The 6^{th} pattern of the artificial nucleic acid comprises ssV_{H}-P4-CD8hinge/TM-41BB-CD3z-P2A-mIL15/Ra-LSP-P2A-endosp-CCL19 in this order from N-terminal.

The 7^{th} pattern of the artificial nucleic acid comprises ssV_{H}-P4-CD8hinge/TM-41BB-CD3z-P2A-IL15Rasp-sushi15-P2A-endosp-CCL19 in this order from N-terminal.

The 8^{th} pattern of the artificial nucleic acid comprises ssV_{H}-P4-CD8hinge/TM-41BB-CD3z-P2A-sIL15-LSP-P2A-endosp-CCL19 in this order from N-terminal.

The 9^{th} pattern of the artificial nucleic acid comprises ssV_{H}-P4-CD8hinge/TM-CD28-CD3z-P2A-IL15sp-sushi15-P2A-endosp-CCL19 in this order from N-terminal.

Additionally, 2 more patterns were prepared using DAP10 and TNFR2 respectively as an intracellular domain between CD8hinge/TM and CD3z.

The 10^{th} pattern of the artificial nucleic acid comprises ssV_{H}-P4-CD8hinge/TM-DAP10-CD3z-P2A-IL2sp-mIL15/Ra in this order from N-terminal.

The 11^{th} pattern of the artificial nucleic acid comprises ssV_{H}-P4-CD8hinge/TM-TNFR2-CD3z-P2A-IL2sp-mIL15/Ra in this order from N-terminal.

### 2. Production of Retrovirus Vector Carrying Anti-Mesothelin-CAR Gene

Phoenix A cells, a retroviral Packaging Cell Line (ATCC), was grown to 50-70% confluency in DMEM 20% FBS and Pen/Strep. DNA complexes were prepared using the respective plasmids containing a pattern of nucleic acids comprising gene encoding the CAR construct described above, the helper plasmids gag-pol and pVSVG and the transduction reagent Fugene HD (Promega) according to manufacturer's protocol. Virus was harvested 16-24 hrs after transfection and was aliquoted and frozen for further use.

### 3. Production of iPS Cell-Derived Anti-Mesothelin-CAR/IL-15γδT Cells

iγδT cells or iγ9δ2T cells were thawed and rested for 3 days in medium (DMEM 10% FCS and additives listed in Table 10. Flasks were coated overnight at 4′C with retronectin (Takara) and CD3 antibody (UCHT1). iγδT cells or iγ9δ2T cells were washed, resuspended in DMEM 10% FCS and supplements specified in Table 9 and transferred to the retronectin and anti-CD3 antibody coated flasks for 72 hours for activation.

iγδT cells or iγ9δ2T cells were transduced with each pattern of nucleic acids comprising gene encoding the CAR construct described above using spinocculation. In brief, iγδT cells or iγ9δ2T cells were transferred retronectin coated plates (prepared according to manufacturer's instructions) and spinocculated (2,000 g; 32′C) with the CAR encoding pVSVG pseudotyped virus described above. The cells were incubated in DMEM 10% FCS and additives described in Table 10 overnight at 37′C 5% CO₂ and transduction was repeated the following day and cells were expanded in DMEM 10% FCS and additives described in Table 10.

The transduced cells were further expanded using retronectin/anti-CD3 antibody activation as described above in DMEM 10% FCS and additives listed in Table 10 and were transferred to G-REX on day 3 after activation. Day 7 after activation cells were harvested and frozen further use. (Note the iPS cell-derived anti-Mesothelin-CAR/IL-15γδT cells prepared from iγδT cells are also referred to herein as “iMeso-CAR/IL-15γδT cells”, and the iPS cell-derived anti-Meso-CAR/IL-15γδT cells prepared from iγ9δ2T cells are also referred to herein as “iMeso-CAR/IL-15γ9δ2T cells”).

### Example 5

Expansion Culture of iMeso-CAR/IL-15γ9δ2T Cells

Using a method similar to that described in Example 3, expansion culture of the iMeso-CAR/IL-15γ9δ2T cells (P4-41BB-mIL15/Ra-CCL19) obtained in Example 5 was performed. A medium containing an additive containing cytokine in Table 9 instead of the additive containing cytokine in Table 4, and an additive containing cytokine in Table 10 instead of the additive containing cytokine in Table 5 was used.

TABLE 9

| product name | manufacturer | Final conc | |
|---|---|---|---|
| Insulin-Transferrin-Selenium | Invitrogen | 1× | |
| Supplements | | | |
| Ascorbic acid 2-phosphate | Sigma | 50 | μg/ml |
| IL-7 | Peprotech | 10 | ng/ml |
| IL-15 | Peprotech | 10 | ng/ml |
| IL-21 | Peprotech | 20 | ng/ml |
| IL-18 | MBL | 50 | ng/ml |
| Z-VAD-FMK | R&D | 10 | μM |

TABLE 10

| product name | manufacturer | Final conc | |
|---|---|---|---|
| Insulin-Transferrin-Selenium | Invitrogen | 1× | |
| Supplements | | | |
| Ascorbic acid 2-phosphate | Sigma | 50 | μg/ml |
| IL-7 | Peprotech | 10 | ng/ml |
| IL-15 | Peprotech | 10 | ng/ml |

Expansion culture of the iMeso-CAR/IL-15γ9δ2T cells obtained in Example 4 was performed.

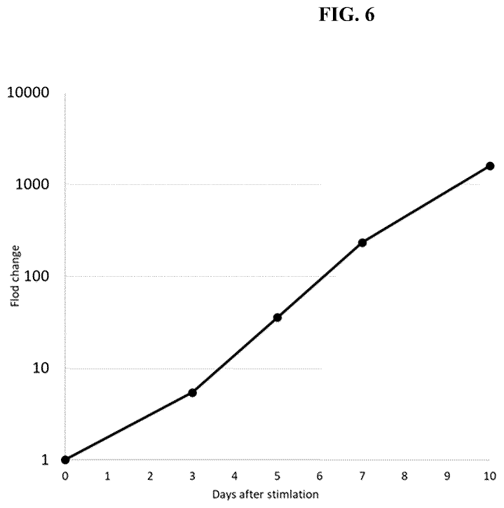

Proliferation of iMeso-CAR/IL-15γ9δ2T cells was observed by stimulation with anti-CD3 antibody (OKT3) (FIG. 6).

### Example 6

### 1. Mesothelin Expression by the Human Cell Lines MiaPaCa-2, MiaPaCa-2 Msln+, Capan-2 and GSU.

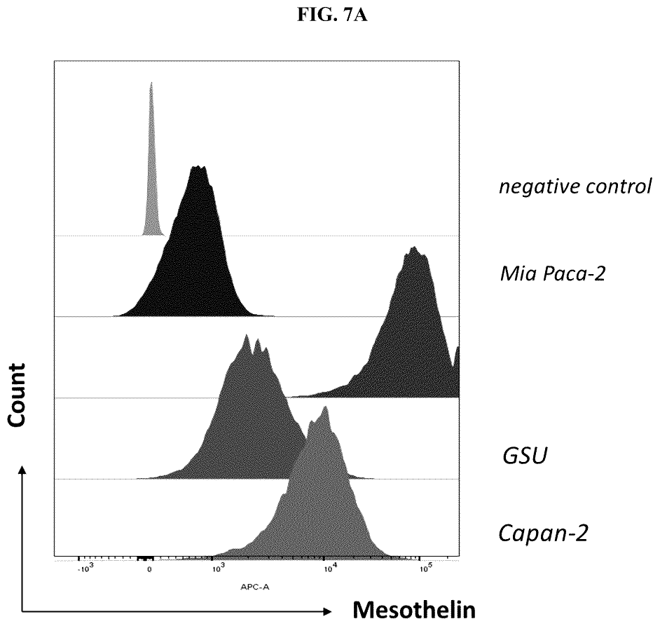

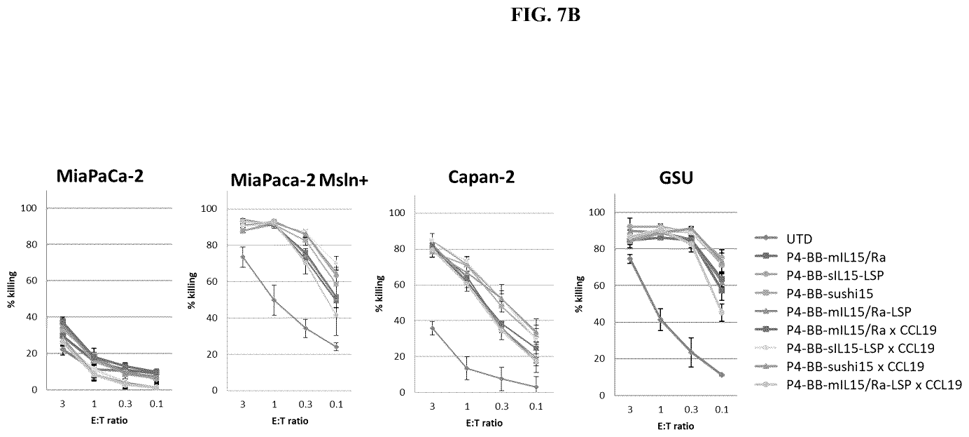

The human cell lines MiaPaCa-2 (ATCC; CRL-1420), MiaPaCa-2 Msln+ (generated from MiaPaca-2 by overexpression of Mesothelin using a viral vector), Capan-2 (ATCC, HTB-80) and GSU (RIKEN, RBC2278) were cultured as recommended by the providers. For flow cytometry cells were harvested using TrypLE Express Enzyme (Gibco; Ser. No. 12/605,010), washed and stained with a primary unlabeled anti-Mesothelin antibody (Rockland; #MN1200301A88) and a secondary APC conjugated anti-mouse IgG antibody (Biolegend; #405308). Dead cells were stained using a fixable viability dye (eBiosciences; #65-0866-14) and cells were fixed before acquisition on a FACS Fortessa (BD Biosciences). Analysis was performed using FlowJo. MiaPaca-2 were very low or negative for Mesothelin expression while Capan-2, GSU and MiaPaca-2 Msln+showed high Mesothelin expression (FIG. 7A).

2. In vitro Cytotoxicity of iPS Cell-Derived Anti-Mesothelin-CAR/IL-15γδT Cells Against the Human Cell Lines MiaPaCa-2, MiaPaCa-2 Msln+, Capan-2 and GSU.

All target cell lines (MiaPaCa-2, MiaPaCa-2 Msln+, Capan-2 and GSU) were detached, washed, resuspended in assay medium and counted before being plated in triplicates on collagen coated flat bottom plates. Cells were incubated for at least 2 hours at 37′C 5% CO₂ to fully adhere.

Meso iCAR-T were thawed, washed, resuspended in assay medium and counted before co-culture with the target cells at the indicated effector to target (E:T) ratio (i.e. 3:1, 1:1, 0.3:1, 0.1:1) for 48 hrs at 37′C 5% CO₂. Killing of target cells by iMeso-CAR/IL-15γ9δ2T cells was assessed using CellTiter-Glo Luminescent Cell Viability Assay (Promega) according to manufacturer's instructions. % killing was calculated according to the following equation:

$$\% killing = \frac{( \begin{matrix}average luminescence coculture - \\ average luminescence effector cells alone\end{matrix} )}{average luminescence target cells alone}$$

The results show that iMeso-CAR/IL-15γ9δ2T cells with all IL-15 versions potently kill cell lines that naturally express Mesothelin (GSU, Capan-2) or that were engineered to express Mesothelin (MiaPaCa-2 Msln+) but not Mesothelin negative MiaPaCa-2 cells in vitro and that killing is highly increased as compared to untransduced control cells (UTD) (iγ9δ2T cells) (FIG. 7B).

### Example 7

1. Assessing the In Vivo Anti-Tumor Efficacy of iPS Cell-Derived Anti-Mesothelin-CAR/IL-15γδT Cells Carrying Different Costimulatory Domains in the Capan-2 s.c. Xenograft Model

6-16 week old female NSG mice (Jackson Labs) were inoculated subcutaneously with 4×10⁶ viable Capan-2 tumor cells in 100ul McCoys 5A+matrigel. Three weeks after implantation the tumor size reached about 50 mm3 and mice were randomized into treatment groups with similar average tumor size (average ˜100 mm3; n=6 per group). The following day 1×10⁶ freshly produced iMeso-CAR/IL-15γ9δ2T cells were injected intratumorally. Body weights were measured twice weekly to monitor toxicity. Tumor size was measured twice weekly and tumor volume was calculated using the formula: tumor volume (mm3)=length×width×height×0.5236. See Table 11 below.

TABLE 11

| | | | | | Cell |
|---|---|---|---|---|---|
| | | | | | number |
| | | | | | (CAR |
| | | | Costimulatory | | positive; |
| Group | Name | Binder | domain | Armoring | *10⁶) |
| 1 | PBS | N/A | N/A | N/A | N/A |
| 2 | P4-41BB- | P4 scFv | 4-1BB | mIL15/Ra | 1 |
| | mIL15/Ra | (VH-VL) | | | |
| 3 | P4-CD28- | P4 scFv | CD28 | mIL15/Ra | 1 |
| | mIL15/Ra | (VH-VL) | | | |
| 4 | P4-DAP10- | P4 scFv | DAP10 | mIL15/Ra | 1 |
| | mIL15/Ra | (VH-VL) | | | |
| 5 | P4-TNFR2- | P4 scFv | TNFR2 | mIL15/Ra | 1 |
| | mIL15/Ra | (VH-VL) | | | |

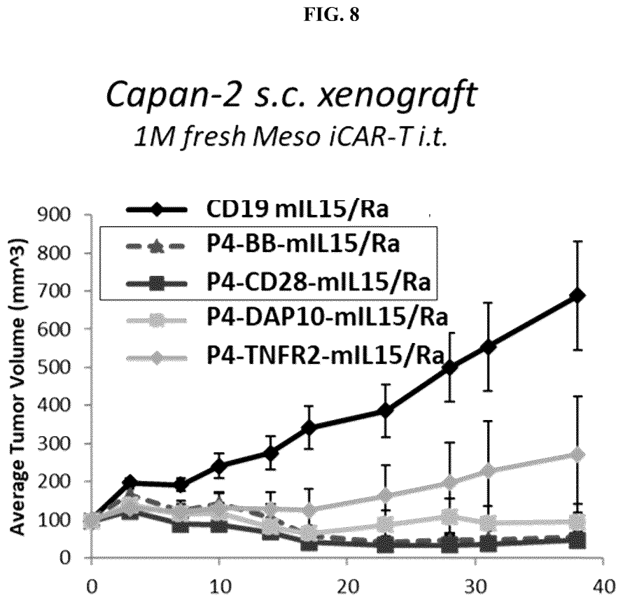

Best anti-tumor efficacy was observed with iMeso-CAR/IL-15γδT cells carrying the 4-1BB or CD28 costimulatory domain (FIG. 8).

### Example 8

1. Assessing the In Vivo Anti-Tumor Efficacy of iPS Cell-Derived Anti-Mesothelin-CAR/IL-15γδT Cells Carrying Different IL-15 Versions and Comparison of 4-1BB and CD28 Costimulatory Domain in the Capan-2 s.c. Xenograft Model

6-16 week old female NSG mice (Jackson Labs; n-6/group) were inoculated subcutaneously with 4×10⁶ viable Capan-2 tumor cells in 100ul McCoys 5A+matrigel. Three weeks after implantation when the tumor size reached about 100 mm3 mice were randomized into treatment groups with similar average tumor size (average ˜100 mm3; n=6 per group). The following day 5×10⁶ (Arm 1) or 1.5×10⁶ (Arm 2) cryopreserved iMeso-CAR/IL-15γ9δ2T cells were injected intratumorally. Body weights were measured twice weekly to monitor toxicity. Tumor size was measured twice weekly and tumor volume was calculated using the formula: tumor volume (mm3)=length×width×height×0.5236. See Tables 12-14.

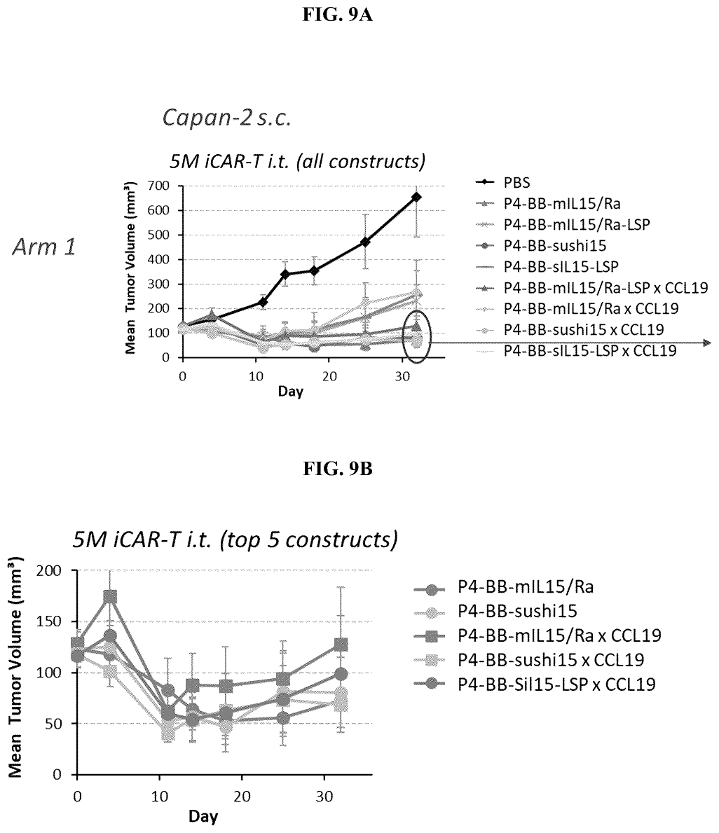

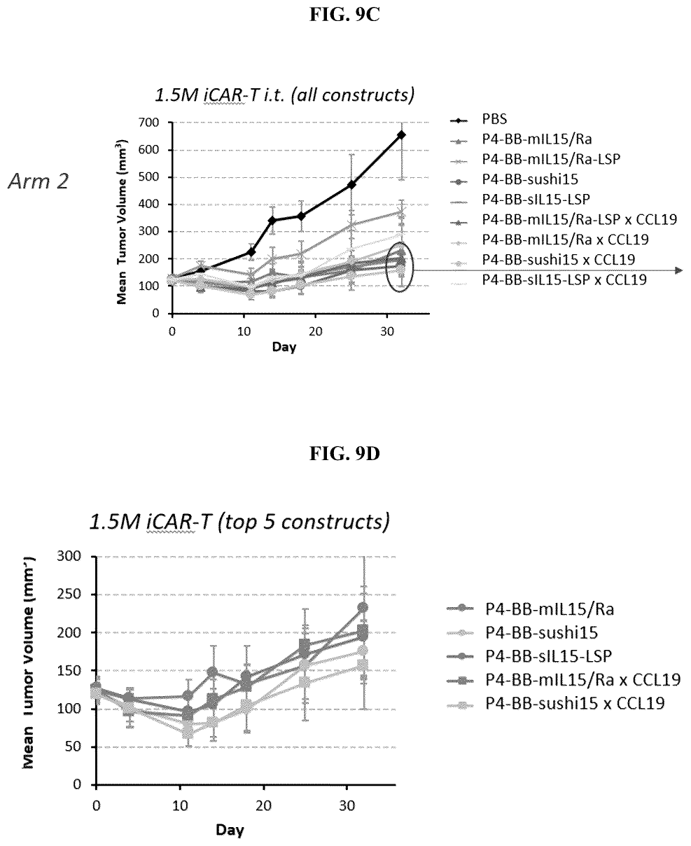

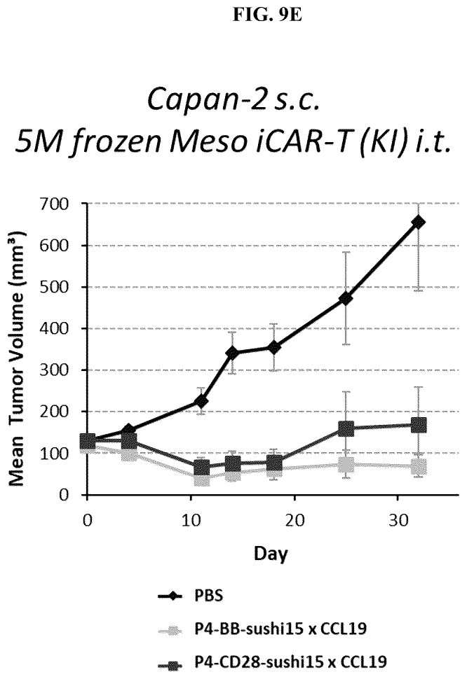

TABLE 12
Arm 1. Also see FIG. 9A and FIG. 9B.

| | | | | | | Cell | |
|---|---|---|---|---|---|---|---|
| | | | | | | number | |
| | | | | | | (CAR | |
| | | | Costim. | Armoring | Armoring | positive; | RANK |
| Group | Name | Binder | domain | 1 | 2 | *10⁶) | (d32) |
| 1 | PBS | N/A | N/A | N/A | N/A | N/A | 9 |
| 2 | P4-41BB- | P4 scFv | 4-1BB | mIL15/Ra | N/A | 5 | 2 |
| | mIL15/Ra | (VH- | | | | | |
| | | VL) | | | | | |
| 3 | P4-41BB- | P4 scFv | 4-1BB | mIL15/Ra- | N/A | 5 | 6 |
| | mIL15/Ra- | (VH- | | LSP | | | |
| | LSP | VL) | | | | | |
| 4 | P4-41BB- | P4 scFv | 4-1BB | Sushi15 | N/A | 5 | 4 |
| | sushi15 | (VH- | | | | | |
| | | VL) | | | | | |
| 5 | P4-41BB- | P4 scFv | 4-1BB | SIL15-LSP | N/A | 5 | 7 |
| | SIL 15-LSP | (VH- | | | | | |
| | | VL) | | | | | |
| 6 | P4-41BB- | P4 scFv | 4-1BB | mIL15/Ra | CCL19 | 5 | 5 |
| | mIL15/Ra- | (VH- | | | | | |
| | CCL19 | VL) | | | | | |
| 7 | P4-41BB- | P4 scFv | 4-1BB | mIL15/Ra- | CCL19 | 5 | 8 |
| | mIL15/Ra- | (VH- | | LSP | | | |
| | LSP- | VL) | | | | | |
| | CCL19 | | | | | | |
| 8 | P4-41BB- | P4 scFv | 4-1BB | Sushi15 | CCL19 | 5 | 1 |
| | sushi15- | (VH- | | | | | |
| | CCL19 | VL) | | | | | |
| 9 | P4-41BB- | P4 scFv | 4-1BB | SIL15- | CCL19 | 5 | 3 |
| | SIL 15-LSP- | (VH- | | LSP | | | |
| | CCL19 | VL) | | | | | |

TABLE 13
Arm 2. Also see FIG. 9C and FIG. 9D.

| | | | | | | Cell | |
|---|---|---|---|---|---|---|---|
| | | | | | | number | |
| | | | | | | (CAR | |
| | | | Costim. | Armoring | Armoring | positive; | RANK |
| Group | Name | Binder | domain | 1 | 2 | *10⁶) | (d32) |
| 1 | PBS | N/A | N/A | N/A | N/A | N/A | 9 |
| 2 | P4-41BB- | P4 scFv | 4-1BB | mIL15/Ra | N/A | 1.5 | 5 |
| | mIL15/Ra | (VH-VL) | | | | | |
| 3 | P4-41BB- | P4 scFv | 4-1BB | mIL15/Ra- | N/A | 1.5 | 8 |
| | mIL15/Ra- | (VH-VL) | | LSP | | | |
| | LSP | | | | | | |
| 4 | P4-41BB- | P4 scFv | 4-1BB | Sushi15 | N/A | 1.5 | 2 |
| | sushi15 | (VH-VL) | | | | | |
| 5 | P4-41BB- | P4 scFv | 4-1BB | SIL15-LSP | N/A | 1.5 | 3 |
| | SIL15-LSP | (VH-VL) | | | | | |
| 6 | P4-41BB- | P4 scFv | 4-1BB | mIL15/Ra | CCL19 | 1.5 | 4 |
| | mIL15/Ra- | (VH-VL) | | | | | |
| | CCL19 | | | | | | |
| 7 | P4-41BB- | P4 scFv | 4-1BB | mIL15/Ra- | CCL19 | 1.5 | 6 |
| | mIL15/Ra- | (VH-VL) | | LSP | | | |
| | LSP- | | | | | | |
| | CCL19 | | | | | | |
| 8 | P4-41BB- | P4 scFv | 4-1BB | Sushi15 | CCL19 | 1.5 | 1 |
| | sushi15- | (VH-VL) | | | | | |
| | CCL19 | | | | | | |
| 9 | P4-41BB- | P4 scFv | 4-1BB | SIL15-LSP | CCL19 | 1.5 | 7 |
| | SIL15-LSP- | (VH-VL) | | | | | |
| | CCL19 | | | | | | |

TABLE 14
Also see FIG. 9E

| | | | | | | Cell | |
|---|---|---|---|---|---|---|---|
| | | | | | | number | |
| | | | | | | (CAR | |
| | | | Costim. | Armoring | Armoring | positive; | RANK |
| Group | Name | Binder | domain | 1 | 2 | *10⁶) | (d32) |
| 1 | PBS | N/A | N/A | N/A | N/A | N/A | 3 |
| 2 | P4-41BB- | P4 scFv | 4-1BB | Sushi15 | CCL19 | 5 | 1 |
| | sushi15- | (VH-VL) | | | | | |
| | CCL19 | | | | | | |
| 3 | P4-CD28- | P4 scFv | CD28 | Sushi15 | CCL19 | 5 | 2 |
| | sushi15- | (VH-VL) | | | | | |
| | CCL19 | | | | | | |

The iMeso-CAR/IL-15γ9δ2T cells carrying the IL-15 versions sushi15 or mIL15/Ra showed overall best anti-tumor efficacy, irrespectively of the used cell number (i.e. 5×10⁶ or 1.5×10⁶; FIG. 9E). iMeso-CAR/IL-15γ9δ2T cells carrying the costimulatory domain 4-1BB showed better tumor control as compared to iMeso-CAR/IL-15γ9δ2T cells using the CD28 costimulatory domain (FIG. 9E).

### Example 9

1. Assessing the In Vivo Anti-Tumor Efficacy of iPS Cell-Derived Anti-Mesothelin-CAR/IL-159982T Cells Armored with Different IL-15 Versions and CCL19 in the GSU s.c. Xenograft Model

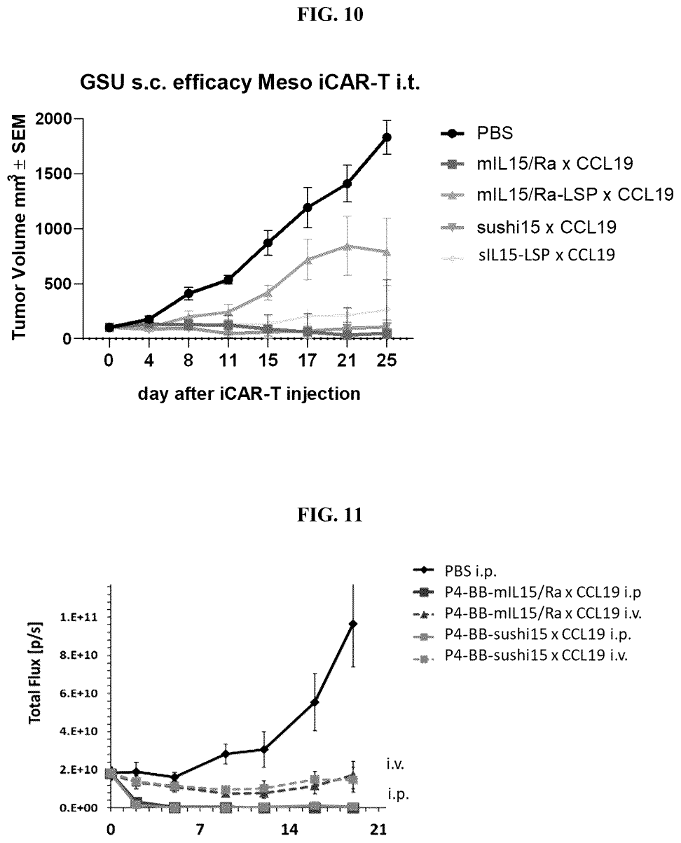

6-16 week old female NSG mice (Jackson Labs; n-6/group) were inoculated subcutaneously with 2×10⁶ viable GSU tumor cells in Matrigel:PBS 1:1. 7 days after implantation the tumor size reached about 100 mm3 and mice were randomized into treatment groups with similar average tumor size (average ˜100 mm3; n=6 per group). The following day 10×10⁶ cryopreserved iMeso-CAR/IL-15γ9δ2T cells were injected intratumorally. Body weights were measured twice weekly to monitor toxicity. Tumor size was measured twice weekly and tumor volume was calculated using the formula: tumor volume (mm3)=length×width×height×0.5236. See Table 15 below and FIG. 10.

TABLE 15

| | | | | | | Cell | |
|---|---|---|---|---|---|---|---|
| | | | | | | number | |
| | | | | | | (CAR | |
| | | | Costim. | Armoring | Armoring | positive; | RANK |
| Group | Name | Binder | domain | 1 | 2 | *10⁶) | (d32) |
| 1 | PBS | N/A | N/A | N/A | N/A | N/A | 5 |
| 2 | P4-41BB- | P4 scFv | 4-1BB | mIL15 | CCL19 | 10 | 1 |
| | mIL15/Ra- | (VH- | | | | | |
| | CCL19 | VL) | | | | | |
| 3 | P4-41BB- | P4 scFv | 4-1BB | mIL15- | CCL19 | 10 | 4 |
| | mIL15/Ra- | (VH- | | LSP | | | |
| | LSP-CCL19 | VL) | | | | | |
| 4 | P4-41BB- | P4 scFv | 4-1BB | Sushi15 | CCL19 | 10 | 2 |
| | sushi15- | (VH- | | | | | |
| | CCL19 | VL) | | | | | |
| 5 | P4-41BB- | P4 scFv | 4-1BB | SIL15- | CCL19 | 10 | 3 |
| | SIL15-LSP- | (VH- | | LSP | | | |
| | CCL19 | VL) | | | | | |

Intraperitoneal administration of 10×10⁶ iMeso-CAR/IL-15γ9δ2T cells cleared tumors while intravenous administration of 10×10⁶ Meso iCAR-T showed tumor control but no complete response. iMeso-CAR/IL-15γ9δ2T cells armored with sushi15-CCL19 or mIL15/Ra-CCL19 performed comparably well.

### Example 10

1. Assessing the In Vivo Anti-Tumor Efficacy of iPS Cell-Derived Anti-Mesothelin-CAR/IL-15γ9δ2T Cells Armored with Different IL-15 Versions and CCL19 in the GSU i.p. Xenograft Model

A luciferized GSU cell line was generated using RediFect Red-FLuc-Puromycin virus from Perkin Elmer (CLS960002). Transduction was performed overnight in the presence of 1.6 μg/ml polybrene (Millipore) cells were transduced with virus carrying a plasmid encoding for human CD19 under control of an EF1a promoter and puromycin resistance. GSU-RedLuc cells were positively selected using puromycin.

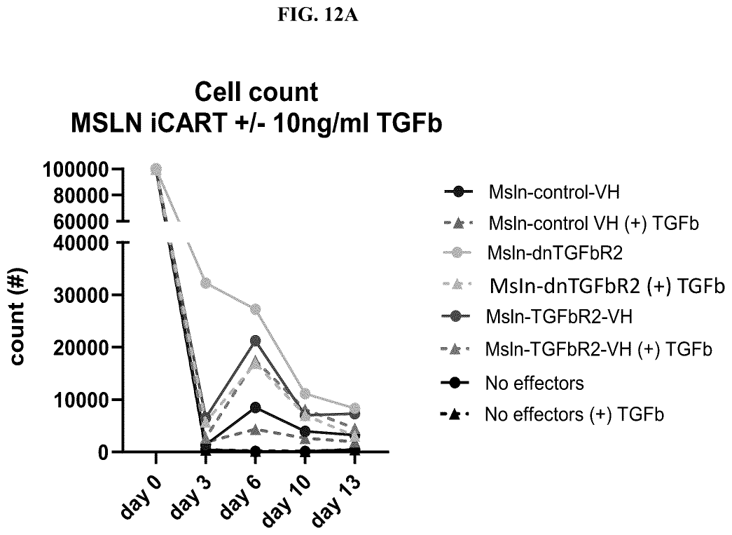

6-16 week old female NSG (Jackson Labs; n=4/group) were intraperitoneally inoculated with 5×10⁶ viable GSU-RedLuc cells in Matrigel:PBS 1:1. Two days after implantation tumor growth was monitored with bioluminescence imaging using the In Vivo Imaging System (IVIS) Spectrum (Perkin Elmer) and analyzed using IVIS imaging software (Perkin Elmer) for randomization. Values are expressed as photons/s. In brief, six minutes before imaging, animals received an intraperitoneal (IP) injection of 150 mg/kg D-Luciferin potassium salt (Promega). Anesthesia was provided with inhaled isoflurane. The day after randomization mice were administered 10×10⁶ viable iMeso-CAR/IL-15γ9δ2T cells intraperitoneally or intravenously and tumor growth and weight were monitored twice per week until the end of the study. See Table 16 below and FIG. 11.

TABLE 16

| | | | | | | Cell | |
|---|---|---|---|---|---|---|---|
| | | | | | | number | |
| | | | | | | (CAR | |
| | | | Costim. | Armoring | Armoring | positive; | |
| Group | Name | Binder | domain | 1 | 2 | *10⁶) | Route |
| 1 | PBS | N/A | N/A | N/A | N/A | N/A | i.p. |
| 2 | P4-41BB- | P4 scFv | 4-1BB | mIL15/Ra | CCL19 | 10 | i.p. |
| | mIL15/Ra- | (VH- | | | | | |
| | CCL19 | VL) | | | | | |
| 3 | P4-41BB- | P4 scFv | 4-1BB | Sushi15 | CCL19 | 10 | i.p. |
| | sushi15- | (VH- | | | | | |
| | CCL19 | VL) | | | | | |
| 4 | P4-41BB- | P4 scFv | 4-1BB | mIL15/Ra | CCL19 | 10 | i.v. |
| | mIL15/Ra- | (VH- | | | | | |
| | CCL19 | VL) | | | | | |
| 5 | P4-41BB- | P4 scFv | 4-1BB | Sushi15 | CCL19 | 10 | i.v. |
| | sushi15- | (VH- | | | | | |
| | CCL19 | VL) | | | | | |

10×10⁶ iMeso-CAR/IL-15γ9δ2T cells potently inhibited tumor growth in the intraperitoneal GSU-RedLuc irrespectively of the used IL-15 version (sushi15 versus mIL15/Ra). Intraperitoneally administered iMeso-CAR/IL-15γ9δ2T cells led to rapid and complete tumor clearance while intravenously injected iMeso-CAR/IL-15γ9δ2T cells significantly inhibited tumor growth as compared to the PBS treated controls.

### Example 11

### Repeat Antigen Stimulation in Mesothelin (Msln) Positive Tumors.

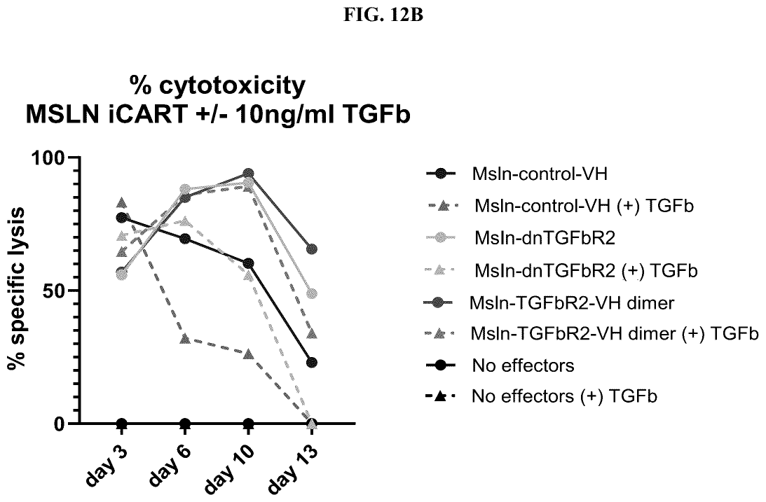

Approximately 100,000 iPSC derived anti-Msln CAR-T cells, co-expressing a CAR against Msln together with a TGFβ modulator (e.g., TGFβR2-VH or dnTGFbR2) or a control VH against GFP (Msln-control V_{H}) were co-cultured in duplicates with 40,000 MiaPaca-2 tumor cells overexpressing human Msln, in the presence or absence of TGF-β (R&D Systems, 10 ng/ml). The TGFβR2-VH was secreted from the CAR-T cell while the dnTGFbR2 was bounded to the membrane of the CAR-T cell. Every 3-4 days, half of the CAR-T cells per well were transferred to a new plate of tumor cells under the same conditions (with or without TGF-β 10 ng/ml). Supernatants were harvested and frozen for later evaluation. CAR-T cells were counted by flow cytometry and FACS phenotyping was performed at selected time-points (FIG. 12A).

Viability of tumor cells was assessed using CellTiterGlo (Promega) according to manufacturer's protocol. The plates were analyzed using a Pherastar plate reader. Percent killing was assessed using the following formula:

$$\% killing = ( 1 - \frac{( signal from tested well )}{signal from confrol wells} ) \times 100$$

The control wells contained tumor cells alone without effector (i.e. CAR-T) cells. The percent cytotoxicity is shown in FIG. 12B.

Cell counts were performed by excluding dead cells using Sytox Red dye (Thermofisher, according to manufacturer's protocol) and equal volumes of cell suspension were acquired on a Fortessa flow cytometer (BD Biosciences) using an HTS unit. Live CAR-T cells were counted by gating on live cells, single cells and size. Results were extrapolated to obtain cell numbers per well.

It was observed that after several rounds of restimulation with target cells, simulating chronic antigen activation, TGF-β induced inhibition of CAR-T cell function (i.e. killing) and inhibited proliferation of CAR-T cells. Only CAR-T cells expressing the TGFβ modulator (e.g., secretion of TGFβR2 VH dimer or expression of membrane bound dnTGFbR2) but not a control VH were protected from the inhibitory effects of TGF-β(10 ng/ml).

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. An isolated nucleic acid molecule comprising a first polynucleotide encoding a chimeric antigen receptor (CAR) comprising an antibody that specifically recognizes human mesothelin, and a 4-1BB intracellular region, and a second polynucleotide encoding an interleukin 15 (IL-15).

2. The isolated nucleic acid molecule of claim 1, wherein the CAR further comprises a CD8 hinge region, a CD8 transmembrane region and a CD3ζintracellular region.

3. (canceled)

4. The isolated nucleic acid molecule of claim 1, further comprising a third polynucleotide encoding CCL19.

5.-6. (canceled)

7. The isolated nucleic acid molecule of claim 1, wherein the antibody comprises a heavy chain variable region (V_{H}) and a light chain variable region (VL), wherein the VH comprises three complementarity-determining regions (CDRs) comprising SEQ ID NOs: 16-18, and wherein the VL comprises three CDRs comprising SEQ ID NOs: 19-21.

8. The isolated nucleic acid molecule of claim 1, wherein the VH comprises SEQ ID NO: 22 and the VL comprises SEQ ID NO: 23.

9. (canceled)

10. The isolated nucleic acid molecule of claim 1, wherein the antibody comprises SEQ ID NO: 1.

11. The isolated nucleic acid molecule of claim 1, wherein the 4-1BB intracellular region comprises SEQ ID NO: 24.

12. The isolated nucleic acid molecule of claim 2, wherein the CD3ζ intracellular region comprises SEQ ID NO: 25.

13. (canceled)

14. The isolated nucleic acid molecule of claim 2, wherein the CD8 hinge region comprises SEQ ID NO: 26.

15. The isolated nucleic acid molecule of claim 2, wherein the CD8 transmembrane region comprises SEQ ID NO: 27.

16.-22. (canceled)

23. The isolated nucleic acid molecule of claim 1, wherein the IL-15 comprises a sequence selected from SEQ ID NOs: 8-11, 28, or 29.

24. (canceled)

25. The isolated nucleic acid molecule of claim 1, wherein the CCL19 comprises SEQ ID NO: 13.

26.-30. (canceled)

31. A vector comprising the nucleic acid molecule of claim 1.

32.-35. (canceled)

36. An immune cell comprising the nucleic acid molecule of claim 1.

37.-38. (canceled)

39. An immune cell expressing:
a) a chimeric antigen receptor (CAR) comprising an antibody that specifically recognizes human mesothelin, and a 4-1BB intracellular region; and
b) IL-15.

40.-45. (canceled)

46. A pharmaceutical composition comprising the immune cell of claim 39, and a pharmaceutically acceptable additive.

47. A method of treating a mesothelin-expressing cancer comprising administering to a subject in need thereof the immune cell of claim 39.

48.-61. (canceled)

62. A method of decreasing tumor cell proliferation comprising contacting the tumor cell with the immune cell of claim 39, thereby decreasing the tumor cell proliferation.

63.-64. (canceled)

65. A method for producing an immune cell expressing cell surface molecules that specifically recognize human mesothelin and IL-15, the method comprising:
introducing the nucleic acid molecule of claim 1 to an immune cell to induce expression of cell surface molecules that specifically recognize human mesothelin and IL-15.

66. (canceled)

67. A kit comprising the nucleic acid molecule of claim 1, and instructions of use.
